(19) United States
(12) Patent Application Publication
(10) Pub. No.: US 2019/0292174 A1
(43) Pub. Date: Sep. 26, 2019

(54) MICROBIOCIDAL OXADIAZOLE DERIVATIVES

(71) Applicant: SYNGENTA PARTICIPATIONS AG, Basel (CH)

(72) Inventors: Daniel STIERLI, Stein (CH); Thomas James HOFFMAN, Stein (CH); Farhan BOU HAMDAN, Stein (CH)

(73) Assignee: SYNGENTA PARTICIPATIONS AG, Basel (CH)

(21) Appl. No.: 16/085,447

(22) PCT Filed: Mar. 14, 2017
(86) PCT No.: PCT/EP2017/056032

§ 371 Date: Sep. 14, 2018

(30) Foreign Application Priority Data
Mar. 15, 2016 (EP) 16160437.6
Apr. 26, 2016 (EP) 16167139.1

(51) Int. Cl. C07D 413/12; C07D 271/06; C07D 413/14; A01N 43/82; A01N 43/84

(52) U.S. Cl. CPC C07D 413/12; C07D 271/06; A01N 43/84; A01N 43/82; C07D 413/14

(57) ABSTRACT

Compounds of the formula (I)

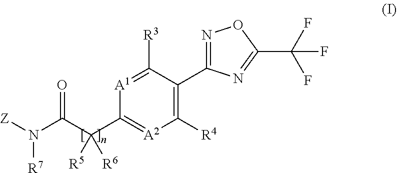

wherein the substituents are as defined in claim 1, useful as a pesticides, especially as fungicides.

### CROSS-REFERENCE TO RELATED APPLICATIONS

This application is a 371 National Stage application of International Application No. PCT/EP2017/056032, filed Mar. 14, 2017, which claims priority to European Patent Application No. 16160437.6 filed Mar. 15, 2016, and European Patent Application No. 16167139.1, filed Apr. 26, 2016, the entire contents of which applications are hereby incorporated by reference.

The present invention relates to microbicidal oxadiazole derivatives, eg, as active ingredients, which have microbicidal activity, in particular, fungicidal activity. The invention also relates to agrochemical compositions which comprise at least one of the oxadiazole derivatives, to processes of preparation of these compounds and to uses of the oxadiazole derivatives or compositions in agriculture or horticulture for controlling or preventing infestation of plants, harvested food crops, seeds or non-living materials by phytopathogenic microorganisms, preferably fungi.

Phenyl oxadiazole derivatives are known as pharmaceutically-active agents from, eg, WO 2013/066835 and WO 20131008162. WO 2015/185485 describes the use of substituted oxadiazoles for combating phytopathogenic fungi. Some phenyl oxadiazole derivatives are also known from chemical libraries.

According to the present invention, there is provided a compound of formula (I):

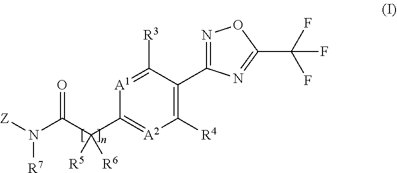

wherein

n represents 0 or 1;

A¹ represents N or CR¹, wherein R¹ is hydrogen, halogen, methyl, ethyl, difluoromethyl, or difluoromethoxy;

A² represents N or CR², wherein R² is hydrogen, halogen, methyl, ethyl, difluoromethyl, or difluoromethoxy;

R³ and R⁴ independently represent hydrogen or halogen;

R⁵ and R⁶ independently represent hydrogen or methyl;

R⁷ represents hydrogen, C₁₋₄alkyl, C₁₋₄alkylcarbonyl, C₁₋₄alkylaminocarbonylC₁₋₄alkyl, di-C₁₋₄ alkylaminocarbonylC₁₋₄alkyl, C₁₋₄alkylaminocarbonyl, phenylcarbonyl, or C₃₋₆cycloalkyl;

Z represents —NR⁸R⁹, wherein

R⁸ represents hydrogen or C₁₋₄alkyl;

R⁹ represents hydrogen, C₁₋₄alkyl, C₃₋₆alkenyl, C₃₋₆alkynyl, C₁₋₄haloalkyl, cyanoC₁₋₄alkyl, hydroxyC₁₋₆alkyl, C₁₋₄alkoxyC₁₋₆alkyl, C₁₋₂fluoroalkoxyC₁₋₆alkyl, C₁₋₄alkylcarbonyl, C₁₋₄alkylcarbonylC₁₋₆alkyl, C₁₋₄haloalkylcarbonyl, C₁₋₄haloalkylaminocarbonyl, C₁₋₄alkoxycarbonyl, C₁₋₄alkoxycarbonylC₁₋₆alkyl, C₁₋₄alkylaminocarbonyl, di-C₁₋₄alkylaminocarbonyl, C₁₋₄alkylaminothiocarbonyl, di-C₁₋₄ alkylaminothiocarbonyl, C₃₋₆cycloalkyl, C₃₋₄cycloalkylC₁₋₃alkyl, phenyl, phenylC₁₋₃alkyl, phenylcarbonyl, heteroaryl, heteroarylC₁₋₃alkyl, heteroarylcarbonyl, wherein the heteroaryl moiety is a 5- or 6-membered aromatic ring which comprises 1, 2, 3 or 4 heteroatoms individually selected from N, O and S, heterocyclyl, heterocyclylC₁₋₃alkyl, wherein the heterocyclyl moiety is a 4- to 6-membered non-aromatic ring which comprises 1, 2 or 3 heteroatoms individually selected from N, O and S, or heterobicyclyl, wherein the heterobicyclyl moiety is a 7- to 11-membered aromatic, saturated or partially saturated fused ring system which comprises 1, 2 or 3 heteroatoms selected from N. O and S. and wherein any of said cycloalkyl, phenyl, heteroaryl, heterocyclyl or heterobicyclyl moieties is optionally substituted by 1, 2, 3 or 4 substituents, which may be the same or different, selected from R¹⁰;

R¹⁰ represents cyano, amino, halogen, hydroxy, C₁₋₄alkyl, methoxy, ethoxy, allyl, propargyl, difluoromethyl, trifluoromethyl, or difluoromethoxy;

or

R⁸ and R⁹, together with the nitrogen atom to which they are attached, form a 5- or 6-membered non-aromatic heterocyclic ring which optionally additionally comprises 1, 2 or 3 heteroatoms individually selected from N, O and S, wherein the heterocyclic ring is optionally substituted by 1, 2, 3, or 4 substituents, which may be the same or different, selected from R¹¹;

or

R⁸ and R⁹, together with the nitrogen atom to which they are attached, form a 7- to 11-membered heteroaromatic or non-aromatic heterocyclic fused ring system which optionally additionally comprises 1, 2 or 3 heteroatoms individually selected from N, O and S, wherein said heteroaromatic or heterocyclic fused ring system is optionally substituted by 1, 2, 3, or 4 substituents, which may be the same or different, selected from R¹¹; and

R¹¹ represents cyano, amino, halogen, hydroxy, C₁₋₄alkyl, C₁₋₂fluoroalkyl, C₁₋₄alkoxy, C₁₋₂ alkoxyC₁₋₄alkyl, allyl, or propargyl and R¹¹ may also represent oxo on a non-aromatic heterocyclic moiety;

or a salt or an N-oxide thereof;

with the proviso that the compound of formula (I) is not:
N′-benzoyl-4-[5-(trifluoromethyl)-1,2,4-oxadiazol-3-yl]benzohydrazide:
1-(2,2,2-trifluoroethyl)-3-[[4-[5-(trifluoromethyl-1,2,4-oxadiazol-3-yl]benzoyl]amino]urea;
N-morpholin-4-yl-4-[5-(trifluoromethyl)-1,2,4-oxadiazol-3-yl]benzamide;
N′-(3-methylphenyl)-4-[5-(trifluoromethyl)-1,2,4-oxadiazol-3-yl]benzohydrazide;
4-methyl-N′4-[4-[5-(trifluoromethyl)-1,2,4-oxadiazol-3-yl]benzoyl]-1,3-thiazole-5-carbohydrazide;
3-methyl-N′-[4-[5-(trifluoromethyl)-1,2,4-oxadiazol-3-yl]benzoyl]thiophene-2-carbohydrazide;
4-[5-(trifluoromethyl)-1,2,4-oxadiazol-3-yl]benzohydrazide; or
4-[5-(trifluoromethyl)-1,2,4-oxadiazol-3-yl]benzohydrazide hydrochloride salt.

Surprisingly, it has been found that the novel compounds of formula (I) have, for practical purposes, a very advantageous level of biological activity for protecting plants against diseases that are caused by fungi.

According to a second aspect of the invention, there is provided an agrochemical composition comprising a fungicidally effective amount of a compound of formula (I). Such an agricultural composition may further comprise at least one additional active ingredient and/or an agrochemically-acceptable diluent or carrier.

According to a third aspect of the invention, there is provided a method of controlling or preventing infestation of useful plants by phytopathogenic microorganisms, wherein a fungicidally effective amount of a compound of formula (I), or a composition comprising this compound as active ingredient, is applied to the plants, to parts thereof or the locus thereof.

According to a fourth aspect of the invention, there is provided the use of a compound of formula (I) as a fungicide. According to this particular aspect of the invention, the use may exclude methods for the treatment of the human or animal body by surgery or therapy.

As used herein, the term “halogen” or “halo” refers to fluorine, chlorine, bromine or iodine, preferably fluorine, chlorine or bromine.

As used herein, the term “C₁₋₆alkyl” refers to a straight or branched hydrocarbon chain radical consisting solely of carbon and hydrogen atoms, containing no unsaturation, having from one to six carbon atoms, and which is attached to the rest of the molecule by a single bond. C₁₋₄alkyl and C₁₋₃ alkyl are to be construed accordingly. Examples of C₁₋₆alkyl include, but are not limited to, methyl, ethyl, n-propyl, 1-methylethyl (iso-propyl), n-butyl, and 1-dimethylethyl (t-butyl). A “C₁-C₆alkylene” group refers to the corresponding definition of C₁-C₆alkyl (and C₁₋₄alkyl), except that such radical is attached to the rest of the molecule by two single bonds. Examples of C₁-C₆alkylene, include, but are not limited to, —CH₂—, —CH₂CH₂— and —(CH₂)₃—.

As used herein, cyano means a —CN group.

As used herein, hydroxy means an —OH group.

As used herein, amino means an —NH₂ group.

As used herein, “formyl” means a —C(O)H group and “acyl” means a —C(O)CH₃ group.

As used herein, the term “C₁₋₄alkoxy” refers to a radical of the formula —ORₐ where Rₐ is a C₁-C₄ alkyl radical as generally defined above. C₁₋₂alkoxy is to be construed accordingly. Examples of C₁₋₄ alkoxy include, but are not limited to, methoxy, ethoxy, propoxy, iso-propoxy, t-butoxy.

As used herein, the term “C₁₋₄haloalkyl” refers to a C₁₋₄alkyl radical as generally defined above substituted by one or more of the same or different halogen atoms. Examples of C₁₋₄haloalkyl include, but are not limited to fluoromethyl, fluoroethyl, difluoromethyl, trifluoromethyl, 2,2,2-trifluoroethyl.

As used herein, the term “C₃₋₆alkenyl” refers to a straight or branched hydrocarbon chain radical group consisting solely of carbon and hydrogen atoms, containing at least one double bond that can be of either the (E)- or (Z)-configuration, having from three to six carbon atoms, which is attached to the rest of the molecule by a single bond. C₃₋₄alkenyl is to be construed accordingly. Examples of C₃-C₆ alkenyl include, but are not limited to, prop-1-enyl, allyl (prop-2-enyl), but-1-enyl.

As used herein, the term “C₃₋₆alkynyl” refers to a straight or branched hydrocarbon chain radical group consisting solely of carbon and hydrogen atoms, containing at least one triple bond, having from three to six carbon atoms, and which is attached to the rest of the molecule by a single bond. The term “C₃₋₄alkynyl” is to be construed accordingly. Examples of C₃₋₆alkynyl include, but are not limited to, prop-1-ynyl, propargyl (prop-2-ynyl), but-1-ynyl.

As used herein, the term “C₁₋₄haloalkoxy” refers to a C₁₋₄alkoxy group as defined above substituted by one or more of the same or different halogen atoms. C₁₋₂haloalkoxy (including C₁₋₂ fluoroalkoxy) is to be construed accordingly. Examples of C₁₋₄haloalkoxy include, but are not limited to, fluoromethoxy, difluoromethoxy, fluoroethoxy, trifluoromethoxy, trifluoroethoxy.

As used herein, the term “C₁₋₄alkoxyC₁₋₆alkyl” refers to radical of the formula R_{b}—O—Rₐ— where R_{b} is a C₁₋₄alkyl radical as generally defined above, and Rₐ is a C₁₋₄alkylene radical as generally defined above. C₁₋₄alkoxyC₁₋₄alkyl and C₁₋₂alkoxyC₁₋₄alkyl are to be construed accordingly.

As used herein, the term “C₁₋₄haloalkoxyC₁₋₆alkyl” refers to radical of the formula R_{b}—O—Rₐ— where R_{b} is a C₁₋₄haloalkyl radical as generally defined above, and Rₐ is a C₁₋₆alkylene radical as generally defined above. C₁₋₂haloalkoxyC₁₋₄alkyl is to be construed accordingly. Examples of C₁₋₄alkoxyC₁₋₆alkyl include, but are not limited to, difluoromethoxymethyl.

As used herein, the term “hydroxyC₁₋₄alkyl” refers to a C₁₋₆alkyl radical as generally defined above substituted by one or more hydroxy groups. HydroxyC₁₋₄alkyl is to be construed accordingly.

As used herein, the term “cyanoC₁₋₆alkyl” refers to refers to a C₁₋₆alkyl radical as generally defined above substituted by one or more cyano groups. CyanoC₁₋₄alkyl is to be construed accordingly.

As used herein, the term “C₁₋₄alkylcarbonyl” refers to a radical of the formula —C(O)Rₐ where Rₐ is a C₁₋₄alkyl radical as generally defined above.

As used herein, the term “C₁₋₄alkylcarbonylC₁₋₄alkyl” refers to a radical of the formula —R_{b}C(O)Rₐ where Rₐ is a C₁₋₄alkyl as generally defined above and R_{b} is a C₁₋₄alkylene radical as generally defined above.

As used herein, the term “C₁₋₄haloalkylcarbonyl” refers to a radical of the formula —C(O)Rₐ where Rₐ is a C₁₋₄haloalkyl as generally defined above.

As used herein, the term “C₁₋₄haloalkylaminocarbonyl” refers to a radical of the formula —C(O)NHRₐ where Rₐ is a C₁₋₄haloalkyl as generally defined above.

As used herein, the term “C₁₋₄alkoxycarbonyl” refers to a radical of the formula —C(O)OR where Rₐ is a C₁₋₄alkyl radical as generally defined above.

As used herein, the term “C₁₋₄alkoxycarbonylC₁₋₄alkyl” refers to a radical of the formula —R_{b}C(O)ORₐ where Rₐ is a C₁₋₄alkyl radical as generally defined above and R_{b} is a C₁₋₄alkylene radical as generally defined above.

As used herein, the term “C₁₋₄alkylaminocarbonyl” refers to a radical of the formula —C(O)NHRₐ where Rₐ is a C₁₋₄alkyl radical as generally defined above.

As used herein, the term “diC₁₋₄alkylaminocarbonyl” refers to a radical of the formula —C(O)NRₐ(Rₐ) where each Rₐ is independently a C₁₋₄alkyl radical as generally defined above.

As used herein, the term “C₁₋₄alkylaminothiocarbonyl” refers to a radical of the formula —C(S)NHRₐ where Rₐ is a C₁₋₄alkyl radical as generally defined above.

As used herein, the term “diC₁₋₄alkylaminothiocarbonyl” refers to a radical of the formula —C(S)NRₐ(Rₐ) where each Rₐ is independently a C₁₋₄alkyl radical as generally defined above.

As used herein, the term “C₃₋₈cycloalkyl” refers to a stable, monocyclic ring radical which is saturated or partially unsaturated and contains 3 to 8 carbon atoms. C₃₋₆cycloalkyl is to be construed accordingly. Examples of C₃₋₈cycloalkyl include, but are not limited to, cyclopropyl, cyclobutyl, cyclopentyl and cyclohexyl.

As used herein, the term “C₃₋₈cycloalkylC₁₋₃alkyl” refers to a C₃₋₈cycloalkyl ring as defined above attached to the rest of the molecule by a C₁₋₃alkyl radical as defined above. The terms “C₃₋₈cycloalkylC₁₋₃alkyl” and “C₃₋₄cycloalkylC₁₋₂alkyl” are to be construed accordingly. Examples of C₃₋₈cycloalkylC₁₋₃alkyl include, but are not limited to cyclopropyl-methyl, cyclobutyl-ethyl, and cyclopentyl-propyl.

As used herein, the term “phenylC₁₋₃alkyl” refers to a phenyl ring attached to the rest of the molecule by a C₁₋₃alkylene radical as defined above. Examples of phenylC₁₋₃alkyl include, but are not limited to, benzyl.

As used herein, the term “phenylcarbonyl” refers to a phenyl ring attached to the rest of the molecule by a C(O) radical.

As used herein, the term “heteroaryl” refers to a 5- or 6-membered monocyclic aromatic ring, or a 7- to 11-membered aromatic fused ring radical which comprises 1, 2, 3 or 4 heteroatoms individually selected from nitrogen, oxygen and sulfur. The heteroaryl radical may be bonded to the rest of the molecule via a carbon atom or heteroatom. Examples of heteroaryl include, furyl, pyrrolyl, thienyl, pyrazolyl, imidazolyl, thiazolyl, isothiazolyl, oxazolyl, isoxazolyl, triazolyl, tetrazolyl, pyrazinyl, pyridazinyl, pyrimidyl, pyridyl, or indolyl.

As used herein, the term “heteroarylC₁₋₃alkyl” refers to a heteroaryl ring as defined above which is attached to the rest of the molecule by a C₁₋₃alkylene radical as defined above.

As used herein the term “heteroarylcarbonyl” refers to a heteroaryl ring as defined above which is attached to the rest of the molecule by a C(O) radical.

As used herein, the term “heterocyclyl” or “heterocyclic” refers to a stable, 4- to 6-, preferably a 5- or 6-membered non-aromatic monocyclic ring, or a 7- to 11-membered non-aromatic fused ring radical which comprises 1, 2, or 3 heteroatoms individually selected from nitrogen, oxygen and sulfur.

The heterocyclyl radical may be bonded to the rest of the molecule via a carbon atom or heteroatom. Examples of heterocyclyl include, but are not limited to, pyrrolinyl, pyrrolidyl, tetrahydrofuryl, tetrahydrothienyl, tetrahydrothiopyranyl, piperidyl, piperazinyl, tetrahydropyranyl, dioxolanyl, morpholinyl, δ-lactamyl, perhydroazepinyl, indolinyl, or benzimidazole.

As used herein, the term “heterocyclylC₁₋₃alkyl” refers to a heterocyclic ring as defined above which is attached to the rest of the molecule by a C₁₋₄alkylene radical as defined above.

The presence of one or more possible asymmetric carbon atoms in a compound of formula (I) means that the compounds may occur in chiral isomeric forms, i.e., enantiomeric or diastereomeric forms. Also atropisomers may occur as a result of restricted rotation about a single bond. Formula (I) is intended to include all those possible isomeric forms and mixtures thereof. The present invention includes all those possible isomeric forms and mixtures thereof for a compound of formula (I). Likewise, formula (I) is intended to include all possible tautomers (including lactam-lactim tautomerism and keto-enol tautomerism) where present. The present invention includes all possible tautomeric forms for a compound of formula (I).

In each case, the compounds of formula (I) according to the invention are in free form, in oxidized form as an N-oxide, in covalently hydrated form, or in salt form, e.g., an agronomically usable or agrochemically acceptable salt form.

N-oxides are oxidized forms of tertiary amines or oxidized forms of nitrogen containing heteroaromatic compounds. They are described for instance in the book “Heterocyclic N-oxides” by A. Albini and S. Pietra, CRC Press, Boca Raton 1991.

Compounds of Formula (I) which are not according to the present invention are:

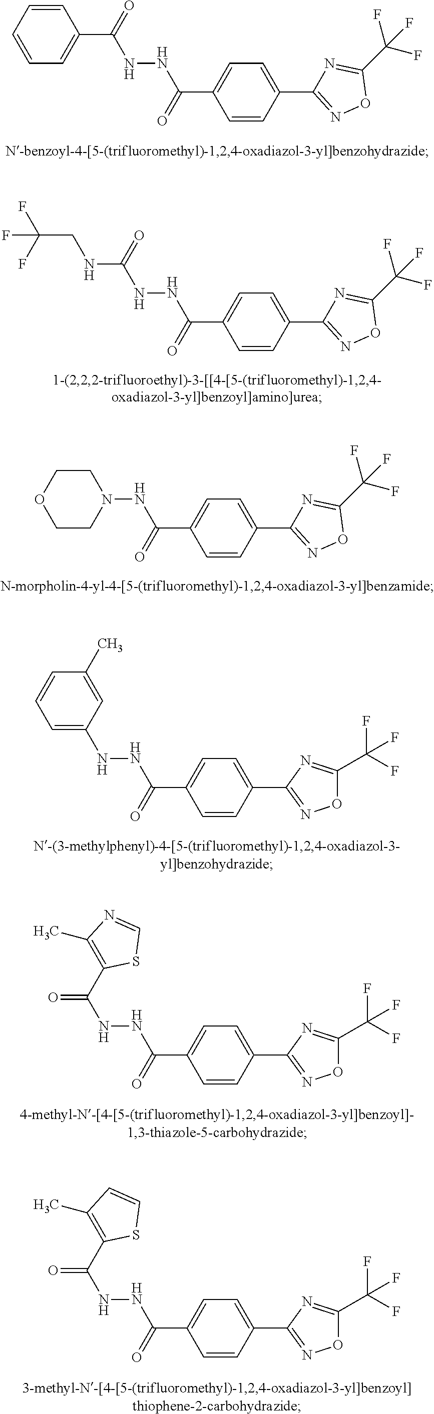 [SMILES: O=C(NNC(=O)c1ccc(-c2noc(C(F)(F)F)n2)cc1)c1ccccc1 ; O=C(NCC(F)(F)F)NNC(=O)c1ccc(-c2noc(C(F)(F)F)n2)cc1 ; O=C(NN1CCOCC1)c1ccc(-c2noc(C(F)(F)F)n2)cc1 ; Cc1cccc(NNC(=O)c2ccc(-c3noc(C(F)(F)F)n3)cc2)c1 ; Cc1ncsc1C(=O)NNC(=O)c1ccc(-c2noc(C(F)(F)F)n2)cc1 ; Cc1ccsc1C(=O)NNC(=O)c1ccc(-c2noc(C(F)(F)F)n2)cc1]

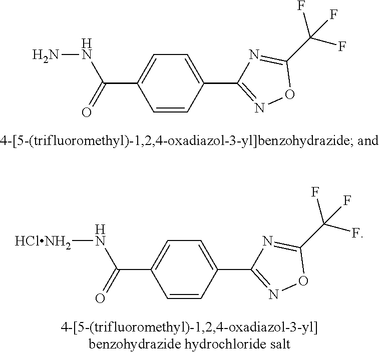

Each of these aforementioned compounds not according to the invention may be used in a method of controlling or preventing infestation of useful plants by phytopathogenic microorganisms, wherein a fungicidally effective amount of the compound or a composition comprising the compound as active ingredient is applied to the plants, to parts thereof or the locus thereof. Likewise, the aforementioned compounds not according to the invention may be useful as fungicidal agents.

The aforementioned compounds not according to the invention are known from the PubChem Compound Database (CID 86777455, CID 86777507, CID 86777680, CID 86812992, CID 86814280, CID 99805418) or Chemical Abstracts (ACS).

The following list provides definitions, including preferred definitions, for substituents n. A¹, A², R¹, R², R³, R⁴, R⁵, R⁶, R⁷, Z, R⁸, R⁹, R¹⁰ and R¹¹, with reference to the compounds of formula (I) according to the invention. For any one of these substituents, any of the definitions given below may be combined with any definition of any other substituent given below or elsewhere in this document,

n represents 0 or 1. In some embodiments of the invention, n is 0. In other embodiments of the invention, n is 1.

A¹ represents N or CR¹, wherein R¹ is hydrogen, halogen, methyl, ethyl, difluoromethyl, or difluoromethoxy. Preferably, A¹ represents N or CR¹, wherein R¹ is selected from hydrogen, halogen or methyl. More preferably, A¹ is CR¹, and R¹ is hydrogen, fluoro or methyl.

A² represents N or CR², wherein R² represents hydrogen, halogen, methyl, ethyl, difluoromethyl, or difluoromethoxy. Preferably, A² represents N or CR₁ wherein R¹ is selected from hydrogen, halogen or methyl. More preferably, A² is CR², and R² is hydrogen, fluoro or methyl, and most preferably, hydrogen.

R³ represents hydrogen or halogen. Preferably, R³ is hydrogen or fluoro.

R⁴ represents hydrogen or halogen. Preferably, R⁴ is hydrogen or fluoro.

In a further embodiment, R³ and R⁴ are both hydrogen.

In one embodiment of the invention, A¹ represents N or CR¹, A² represents CR², and 0, 1 or 2 of R¹, R², R³ and R⁴ are fluorine, wherein when any of R¹, R², R³ and R⁴ is not fluorine, it is hydrogen.

R⁵ and R⁶ independently represent hydrogen or methyl. Preferably. R⁵ and R⁶ are both hydrogen.

R⁷ is hydrogen, C₁₋₄alkyl, C₁₋₄alkylcarbonyl, C₁₋₄alkylaminocarbonylC₁₋₄alkyl, di-C₁₋₄ alkylaminocarbonylC₁₋₄alkyl, C₁₋₄alkylaminocarbonyl, phenylcarbonyl, or C₃₋₆cycloalkyl. Preferably. R⁷ is hydrogen, C₁₋₄alkyl, C₁₋₄alkylaminocarbonylC₁₋₄alkyl or phenylcarbonyl. More preferably, R⁷ is hydrogen, methyl, N-tert-butylacetamide, or phenylcarbonyl. Most preferably, R⁷ is hydrogen.

Z represents —NR⁸R⁹.

R⁸ represents hydrogen or C₁₋₄alkyl. Preferably, R⁸ is hydrogen or methyl, in particular hydrogen.

R⁹ represents hydrogen, C₁₋₆alkyl, C₃₋₆alkenyl, C₃₋₆alkynyl, C₁₋₄haloalkyl, cyanoC₁₋₆alkyl, hydroxylC₁₋₆alkyl, C₁₋₄alkoxyC₁₋₆alkyl, C₁₋₂fluoroalkoxyC₁₋₆alkyl, C₁₋₄alkylcarbonyl, C₁₋₄alkylcarbonylC₁₋₆ alkyl, C₁₋₄haloalkylcarbonyl, C₁₋₄haloalkylaminocarbonyl, C₁₋₄alkoxycarbonyl, C₁₋₄alkoxycarbonylC₁₋₆alkyl, C₁₋₄alkylaminocarbonyl, di-C₁₋₄alkylaminocarbonyl, C₁₋₄alkylaminothiocarbonyl, di-C₁₋₄ alkylaminothiocarbonyl, C₃₋₈cycloalkyl, C₃₋₈cycloalkylC₁₋₃alkyl, phenyl, phenylC₁₋₃alkyl, phenylcarbonyl, heteroaryl, heteroarylC₁₋₃alkyl, heteroarylcarbonyl, wherein the heteroaryl moiety is a 5- or 6-membered aromatic ring which comprises 1, 2, 3 or 4 heteroatoms individually selected from N, O and S, heterocyclyl, heterocyclylC₁₋₃alkyl, wherein the heterocyclyl moiety is a 4- to 6-membered non-aromatic ring which comprises 1, 2 or 3 heteroatoms individually selected from N, O and S, or heterobicyclyl, wherein the heterobicyclyl moiety is a 7- to 11-membered aromatic, saturated or partially saturated fused ring system which comprises 1, 2 or 3 heteroatoms selected from N, O and S, and wherein any of said cycloalkyl, phenyl, heteroaryl, heterocyclyl or heterobicyclyl moieties is optionally substituted by 1, 2, 3 or 4 substituents, which may be the same or different, selected from R¹⁰.

Preferably, R⁹ represents C₁₋₆alkyl, C₃₋₆alkenyl, C₃₋₆alkynyl, C₁₋₄haloalkyl, cyanoC₁₋₆alkyl, hydroxylC₁₋₆alkyl, C₁₋₄alkoxyC₁₋₆alkyl, C₁₋₂fluoroalkoxyC₁₋₆alkyl, C₁₋₄alkylcarbonyl, C₁₋₄alkylcarbonylC₁₋₆alkyl, C₁₋₄haloalkylcarbonyl, C₁₋₄haloalkylaminocarbonyl, C₃₋₄haloalkylaminocarbonyl, C₁₋₄ alkoxycarbonyl, C₁₋₄alkoxycarbonylC₁₋₄alkyl, C₁₋₄alkylaminocarbonyl, di-C₁₋₄alkylaminocarbonyl, C₁₋₄alkylaminothiocarbonyl, di-C₁₋₄alkylaminothiocarbonyl, C₃₋₈cycloalkyl, C₃₋₈cycloalkylC₁₋₃alkyl, phenylC₁₋₃alkyl, heteroaryl, heteroarylC₁₋₃alkyl, heteroarylcarbonyl, wherein the heteroaryl moiety is a 5-membered aromatic ring which comprises 1, 2, 3 or 4 heteroatoms individually selected from N and O, or a 6-membered aromatic ring which comprises 1, 2, 3 or 4 heteroatoms individually selected from N, O and S, heterocyclyl, heterocyclylC₁₋₄alkyl, wherein the heterocyclyl moiety is a 4- to 6-membered non-aromatic ring which comprises 1, 2 or 3 heteroatoms individually selected from N, O and S, or heterobicyclyl, wherein the heterobicyclyl moiety is a 7- to 11-membered aromatic, saturated or partially saturated fused ring system which comprises 1, 2 or 3 heteroatoms selected from N, O and S, and wherein any of said cycloalkyl, phenyl, heteroaryl, heterocyclyl or heterobicyclyl moieties are optionally substituted by 1, 2, 3 or 4 substituents, which may be the same or different, selected from R¹⁰.

More preferably, R⁹ represents hydrogen. C₁₋₄alkyl, hydroxylC₁₋₄alkyl, C₁₋₄alkoxyC₁₋₆alkyl, C₁₋₂ fluoroalkoxyC₁₋₆alkyl, C₁₋₄alkylcarbonyl, C₁₋₄alkoxycarbonyl. C₃₋₈cycloalkyl, and C₃₋₈cycloalkylC₁₋₃alkyl. Even more preferably R⁹ is hydrogen, C₁₋₆alkyl, C₁₋₄alkylcarbonyl, C₁₋₄alkoxycarbonyl, hydroxylC₁₋₆ alkyl, or C₁₋₂fluoroalkoxyC₁₋₆alkyl. Further more preferably, R⁹ is hydrogen, methyl, acyl, methoxycarbonyl, 2-hydroxyethyl, or difluoromethoxyethyl.

In some embodiments of R⁹, any of the cycloalkyl, phenyl, heteroaryl or heterocyclyl moieties may be optionally substituted by 1 or 2 substituents, which may be the same or different, selected from R¹⁰.

Alternatively, R⁸ and R⁹, together with the nitrogen atom to which they are attached, form a 5- or 6-membered non-aromatic heterocyclic ring which optionally additionally comprises 1, 2 or 3 heteroatoms individually selected from N, O and S, wherein the heterocyclic ring is optionally substituted by 1, 2, 3, or 4 substituents, which may be the same or different, selected from R¹¹, or

R⁸ and R⁹, together with the nitrogen atom to which they are attached, form a 7- to 11-membered heteroaromatic or non-aromatic heterocyclic fused ring system which optionally additionally comprises 1, 2 or 3 heteroatoms individually selected from N. O and S, wherein said heteroaromatic or heterocyclic fused ring system is optionally substituted by 1, 2, 3, or 4 substituents, which may be the same or different, selected from R¹¹.

Preferably, R⁸ and R⁹, together with the nitrogen atom to which they are attached, form an oxazolidine, imidazoline, piperidine, morpholine, indole, indoline or benzimidazole group, which is optionally substituted by 1 or 2 substituents selected from R¹¹, which may be the same or different. More preferably, R⁸ and R⁹, together with the nitrogen atom to which they are attached, form a 6-lactam, imidazolinone, imidazolin-dione, oxazolidinone, or morpholine, which is optionally substituted by 1 or 2 substituents selected from R¹¹, which may be the same or different, wherein R¹¹ is selected from cyano, amino, halogen, hydroxy, and C₁₋₄alkyl. Even more preferably, R⁸ and R⁹, together with the nitrogen atom to which they are attached, form a δ-lactam, imidazolinone, imidazolin-dione, oxazolidinone or morpholine group, which is optionally substituted by 1 or 2 substituents selected from R¹¹, which may be the same or different, wherein R¹¹ is selected from cyano, amino, halogen, hydroxy, and C₁₋₄alkyl. More preferably still, R⁸ and R⁹, together with the nitrogen atom to which they are attached, form a δ-lactam, oxazolidinone or morpholine group.

In some embodiments of the invention, when R⁸ and R⁹, together with the nitrogen atom to which they are attached form a 5- or 6-membered non-aromatic heterocyclic ring which optionally additionally comprises 1, 2 or 3 heteroatoms individually selected from N, O and S (or 1 or 2 heteroatoms individually selected from N, O and S, or a single heteroatom selected from N, O and S), the heterocyclic ring may be optionally substituted by 1 or 2 substituents, which may be the same or different, selected from R¹¹.

In some embodiments of the invention, when R⁸ and R⁹, together with the nitrogen atom to which they are attached, form a 7- to 11-membered heteroaromatic or non-aromatic heterocyclic fused ring system which optionally additionally comprises 1, 2 or 3 heteroatoms individually selected from N, O and S, the heteroaromatic or heterocyclic fused ring system may be optionally substituted by 1 or 2 substituents, which may be the same or different, selected from R¹¹.

In some embodiments of the invention, when n is 0: R⁹ is not hydrogen or C₂-fluoroalkylaminocarbonyl; R⁹ is not monomethyl-substituted phenyl or phenylcarbonyl, or is not a monomethyl-substituted heteroarylcarbonyl moiety comprising a 5-membered heteroaromatic ring comprising a sulfur atom; or R⁸ and R⁹, together with the nitrogen atom to which they are attached, do not form an unsubstituted morpholinyl.

R¹⁰ represents cyano, amino, halogen, hydroxy, C₁₋₄alkyl, methoxy, ethoxy, allyl, propargyl, difluoromethyl, trifluoromethyl, or difluoromethoxy. Preferably, R¹⁰ represents amino, halogen, hydroxy, or C₁₋₄alkyl. More preferably, R¹⁰ represents halogen or C₁₋₄alkyl. Even more preferably, R¹⁰ represents fluoro or methyl.

R¹¹ represents hydrogen, cyano, amino, halogen, hydroxy, C₁₋₄alkyl, C₁₋₂fluoroalkyl, C₁₋₄ alkoxy, C₁₋₂alkoxyC₁₋₄alkyl, allyl, or propargyl. R¹¹ may also represent an oxo (eg, a single oxo) on a heterocyclic moiety. Preferably, R¹¹ is hydrogen, cyano, halogen, C₁₋₄alkyl, C₁₋₄alkoxy, or R¹¹ may be oxo when present on a heterocyclic moiety. More preferably, R¹¹ is methyl, or R¹¹ may be oxo when present on a heterocyclic moiety.

Preferably, the compound according to Formula (I) is selected from a compound 1.1 to 1.32 listed in Table T1 (below).

Preferably, in a compound according to formula (I) of the invention, n is 0 or 1;
A¹ represents N or CR¹, wherein R¹ is selected from hydrogen, halogen, or methyl;
A² represents CR², wherein R² is hydrogen, halogen or methyl;
R³ and R⁴ both represent hydrogen;
R⁵ and R⁶ both represent hydrogen;
R⁷ is selected from hydrogen, C₁₋₄alkyl, C₁₋₄alkylcarbonyl, C₁₋₄alkylaminocarbonylC₁₋₄alkyl or phenylcarbonyl;
Z is —NR⁸R⁹, wherein
R⁸ represents hydrogen or methyl; and
R⁹ is selected from hydrogen, C₁₋₄alkyl, hydroxylC₁₋₄alkyl, C₁₋₄alkoxyC₁₋₄alkyl, C₁₋₂fluoroalkoxyC₁₋₄ alkyl, C₁₋₄alkylcarbonyl, C₁₋₄alkoxycarbonyl, C₃₋₆cycloalkyl, or C₃₋₆cycloalkylC₁₋₃alkyl.

More preferably, n is 0;
A¹ represents CR¹, wherein R¹ is hydrogen;
A² represents CR^{Z}, wherein R² is hydrogen;
R³ and R⁴ both represent hydrogen;
R⁷ is selected from hydrogen, C₁₋₄alkyl, C₁₋₄alkylcarbonyl, C₁₋₄alkylaminocarbonylC₁₋₄alkyl or phenylcarbonyl;
Z is —NR⁸R⁹; wherein
R⁸ represents hydrogen or methyl; and
R⁹ is selected from hydrogen, C₁₋₄alkyl, hydroxylC₁₋₄alkyl, C₁₋₄alkoxyC₁₋₄alkyl, C₁₋₂fluoroalkoxyC₁₋₄alkyl, C₁₋₄alkylcarbonyl, C₁₋₄alkoxycarbonyl, C₃₋₆cycloalkyl, or C₃₋₆cycloalkylC₁₋₄alkyl.

Preferably, in a compound according to formula (I) of the invention, n is 0 or 1;
A¹ represents N or CR¹, wherein R¹ is selected from hydrogen, halogen, or methyl;
A² represents CR², wherein R² is hydrogen, halogen or methyl;
R³ and R⁴ both represent hydrogen;
R⁵ and R⁶ both represent hydrogen;
R⁷ is selected from hydrogen, C₁₋₄alkylaminocarbonylC₁₋₄alkyl or phenylcarbonyl; and
Z is —NR⁸R⁹, wherein
R⁸ and R⁹ together with the nitrogen atom to which they are attached, form a δ-lactam, imidazolinone, imidazolin-dione, oxazolidinone, or morpholine, which is optionally substituted by 1 or 2 substituents, which may be the same or different, selected from cyano, amino, halogen, hydroxy, and C₁₋₄alkyl.

More preferably, in a compound according to formula (I) of the invention, n is 0 or 1;
A¹ represents N or CR¹, wherein R¹ is selected from hydrogen, fluoro, or methyl;
A² represents CR², wherein R² is hydrogen, fluoro or methyl;
R³ and R⁴ both represent hydrogen;
R⁵ and R⁶ both represent hydrogen;
R⁷ is selected from hydrogen;
Z is —NR⁸R⁹, wherein
R⁸ and R⁹ together with the nitrogen atom to which they are attached, form a δ-lactam, imidazolinone, imidazolin-dione, oxazolidinone, or morpholine, which is optionally substituted by 1 or 2 substituents, which may be the same or different, selected from cyano, amino, halogen, hydroxy, and C₁₋₄alkyl.

Preferably, in a compound according to formula (I) of the invention, n is 0 or 1;
A¹ represents N or CR¹, wherein R¹ is selected from hydrogen, halogen, or methyl;
A² represents CR², wherein R² is hydrogen, halogen or methyl;
R³ and R⁴ both represent hydrogen;
R⁵ and R⁶ both represent hydrogen;
R⁷ is selected from hydrogen, C₁₋₄alkylaminocarbonylC₁₋₄alkyl or phenylcarbonyl; and
Z is —NR⁸R⁹, wherein
R⁸ and R⁹ together with the nitrogen atom to which they are attached form an indole or indolene, which is optionally substituted by 1 or 2 substituents, which may be the same or different, selected from cyano, amino, halogen, hydroxy, and C₁₋₄alkyl.

More preferably, in a compound according to formula (I) of the invention, n is 0;
A¹ represents N or CR¹, wherein R¹ is selected from hydrogen, halogen, or methyl;
A² represents CR², wherein R² is hydrogen;
R³ and R⁴ both represent hydrogen;
R⁷ is hydrogen;
Z is —NR⁸R⁹, wherein
R⁸ and R⁹ together with the nitrogen atom to which they are attached form an indole or indolene, which is optionally substituted by 1 or 2 substituents, which may be the same or different, selected from cyano, amino, halogen, hydroxy, and C₁₋₄alkyl.

In a further embodiment of the compounds of the invention, n is 0 or 1;
A¹ represents CR¹, wherein R¹ is hydrogen or fluoro;
A² represents CR², wherein R² s hydrogen;
R³ and R⁴ both represent hydrogen, or R³ is fluoro and R⁴ is hydrogen;
R⁵ and R⁶ both represent hydrogen
R⁷ is hydrogen;
R⁸ represents hydrogen or methyl;
R⁹ is selected from methyl or difluoromethoxyethyl, or R⁸ and R⁹, together with the nitrogen atom to which they are attached, form a δ-lactam, oxazolidinone or morpholine group, which is optionally substituted by 1 or 2 substituents, which may be the same or different, selected from cyano, amino, halogen, hydroxy, and C₁₋₄alkyl.

The compounds of the present invention may be enantiomers of the compound of Formula (I) as represented by a Formula (Ia) or a Formula (Ib), wherein R⁵ and R⁶ are different.

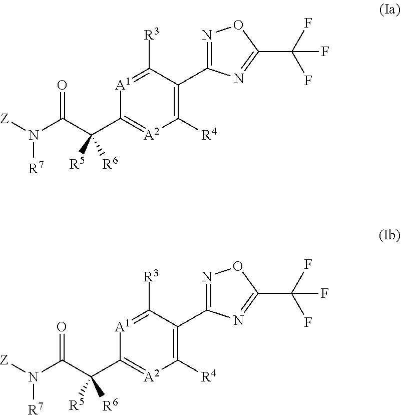

It is understood that when in aqueous media, the compounds of formula (I) according to the invention may be present in a reversible equilibrium with the corresponding covalently hydrated forms (ie, the compounds of formula (I-I) and formula (I-II) as shown below) at the CF₃-oxadiazole motif. This dynamic equilibrium may be important for the biological activity of the compounds of Formula (I). The designations of n, A¹, A², R¹, R², R³, R⁴, R⁵, R⁶, R⁷, Z, R⁸, R⁹, R¹⁰ and R¹¹, with reference to the compounds of formula (I) of the present invention apply generally to the compounds of Formula (I-I) and Formula (I-II), as well as to the specific disclosures of combinations of n, A¹, A², R¹, R², R³, R⁴, R⁵, R⁶, R⁷, Z, R⁸, R⁹, R¹⁰ and R¹¹, as represented in Tables 1.1 to 1.19, below, or the compounds 1.1 to 1.32 described in Table T1 (below).

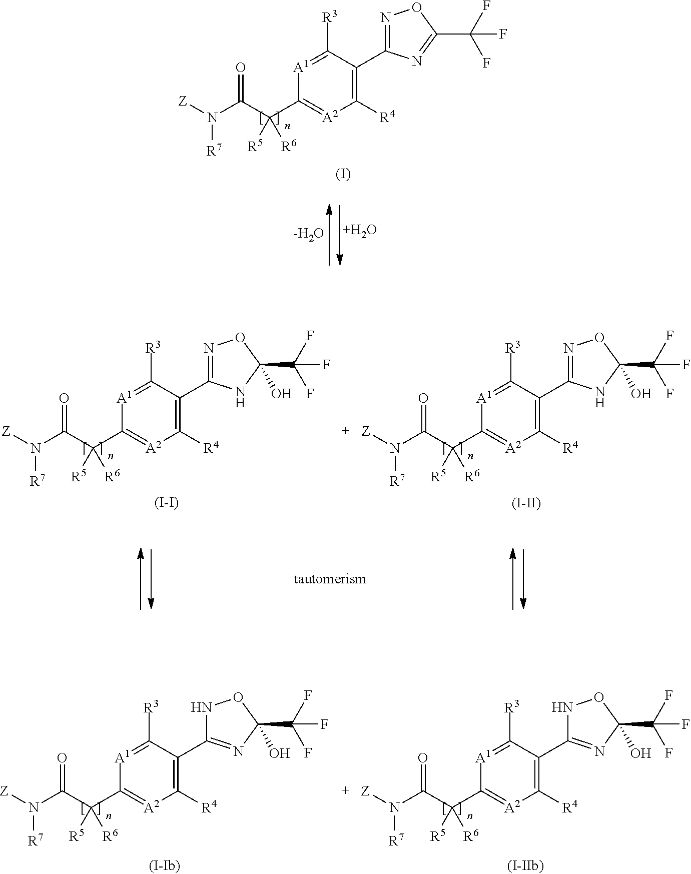

Compounds of the present invention can be made as shown in the following schemes, in which, unless otherwise stated, the definition of each variable is as defined above for a compound of formula (I).

The compounds of formula (I) can be obtained by an amide coupling transformation with compounds of formula (II) and compounds of formula (III) by activating the carboxylic acid function of the compounds of formula (III), a process that usually takes place by converting the —OH of the carboxylic acid into a good leaving group, such as a chloride group, for example by using (COCl)₂ or SOCl₂, prior to treatment with the compounds of formula (II), preferably in a suitable solvent (eg, dimethylformamide, dichloromethane or tetrahydrofuran), preferably at a temperature of between 25° C. and 100° C., and optionally in the presence of a base such as triethylamine or N,N-diisopropylethylamine, or under conditions described in the literature for an amide coupling. For examples, see Valeur. E.; Bradley. M. Chem. Soc. Rev. (2009), 38, 606 and Chinchilla, R., Najera. C. Chem. Soc. Rev. (2011), 40, 5084. This is shown in Scheme 1 below. Compounds of formula (III) can be made by known methods from known compounds or are commercially available. For examples, see: Liu, K. et al. J. Med. Chem. (2008), 51, 7843 and WO 2013/008162 A1. Compounds of formula (II) are known compounds or are commercially available.

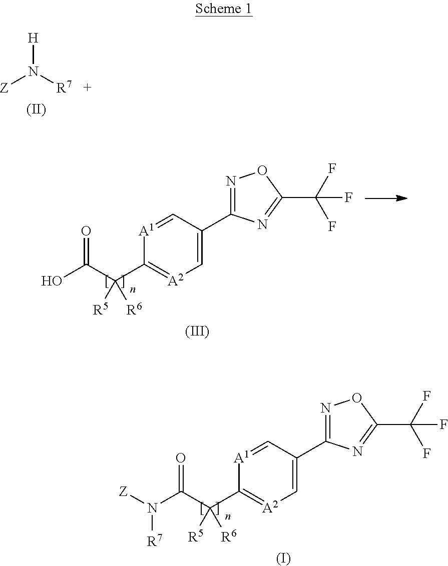

Alternatively, compounds of formula (I) can be prepared from compounds of formula (IV) by treatment with trifluoroacetic anhydride in a suitable solvent, such as tetrahydrofuran, at a temperature between 0° C., and 25° C. For related examples, see Kitamura, S. et al. Chem. Pharm. Bull. (2001), 49, 268. This is shown in Scheme 2.

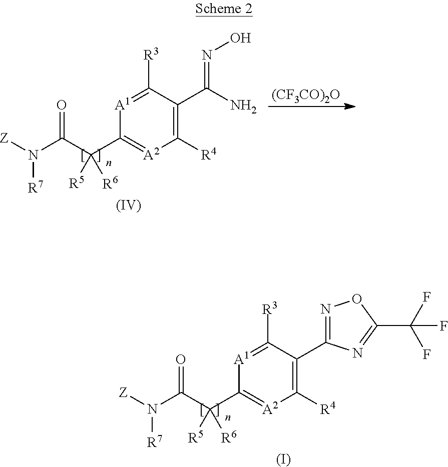

Compounds of formula (VI) can be prepared from compounds of formula (V) by treating them with a hydroxylamine hydrochloride salt in the presence of a base, such as sodium carbonate, in a suitable solvent, such as methanol, at a temperature between 0° C., and 100° C. For related examples, see Kitamura, S. et al. Chem. Pharm. Bull. (2001), 49, 268. This s shown in Scheme 3.

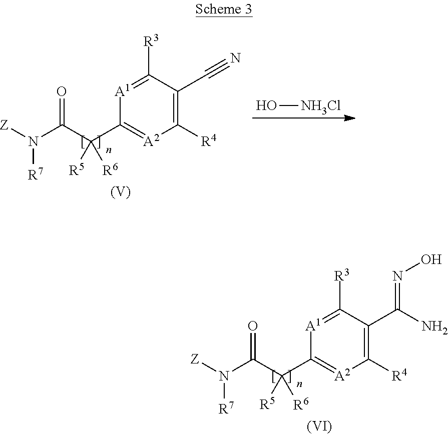

Compounds of formula (III) can be prepared from compounds of formula (VII) by treatment with trifluoroacetic anhydride in a suitable solvent, such as tetrahydrofuran, at a temperature between 0° C. and 25° C. For related examples, see Kitamura, S. et al. Chem. Pharm. Bull. (2001), 49, 268. This is shown in Scheme 4.

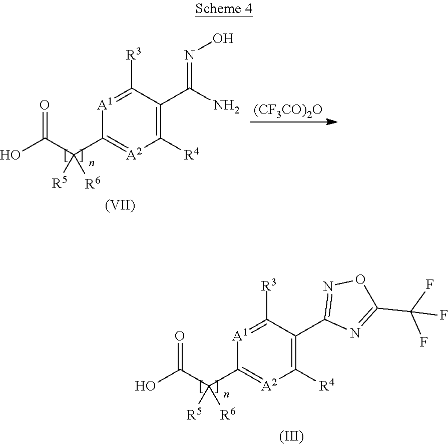

Compounds of formula (VII) can be prepared from compounds of formula (VIII) by treating them with a hydroxylamine hydrochloride salt in the presence of a base, such as sodium carbonate, in a suitable solvent, such as methanol, at a temperature between 0° C. and 100° C. Compounds of formula (VIII) are commercially available. For related examples, see Kitamura, S. at al. Chem. Pharm. Bull. (2001), 49, 268. This is shown in Scheme 5.

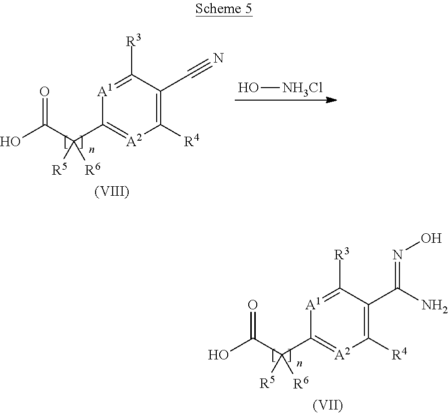

As already indicated, surprisingly, it has now been found that the novel compounds of formula (I) of the present invention have, for practical purposes, a very advantageous level of biological activity for protecting plants against diseases that are caused by fungi.

The compounds of formula (I) can be used in the agricultural sector and related fields of use, e.g., as active ingredients for controlling plant pests or on non-living materials for the control of spoilage microorganisms or organisms potentially harmful to man. The novel compounds are distinguished by excellent activity at low rates of application, by being well tolerated by plants and by being environmentally safe. They have very useful curative, preventive and systemic properties and can be used for protecting numerous cultivated plants. The compounds of formula (I) can be used to inhibit or destroy the pests that occur on plants or parts of plants (fruit, blossoms, leaves, stems, tubers, roots) of different crops of useful plants, while at the same time protecting also those parts of the plants that grow later, e.g., from phytopathogenic microorganisms.

The present invention further relates to a method for controlling or preventing infestation of plants or plant propagation material and/or harvested food crops susceptible to microbial attack by treating plants or plant propagation material and/or harvested food crops wherein an effective amount a compound of formula (I) is applied to the plants, to parts thereof or the locus thereof.

It is also possible to use compounds of formula (I) as fungicide. The term “fungicide” as used herein means a compound that controls, modifies, or prevents the growth of fungi. The term “fungicidally effective amount” where used means the quantity of such a compound or combination of such compounds that is capable of producing an effect on the growth of fungi. Controlling or modifying effects include all deviation from natural development, such as killing, retardation and the like, and prevention includes barrier or other defensive formation in or on a plant to prevent fungal infection.

It may also be possible to use compounds of formula (I) as dressing agents for the treatment of plant propagation material, e.g., seed, such as fruits, tubers or grains, or plant cuttings, for the protection against fungal infections as well as against phytopathogenic fungi occurring in the soil. The propagation material can be treated with a composition comprising a compound of formula (I) before planting: seed, for example, can be dressed before being sown. The active compounds of formula (I) can also be applied to grains (coating), either by impregnating the seeds in a liquid formulation or by coating them with a solid formulation. The composition can also be applied to the planting site when the propagation material is being planted, for example, to the seed furrow during sowing. The invention relates also to such methods of treating plant propagation material and to the plant propagation material so treated.

Furthermore, the compounds of formula (I) can be used for controlling fungi in related areas, for example in the protection of technical materials, including wood and wood related technical products, in food storage, in hygiene management.

In addition, the invention could be used to protect non-living materials from fungal attack, e.g. lumber, wall boards and paint.

The compounds of formula (I) are for example, effective against fungi and fungal vectors of disease as well as phytopathogenic bacteria and viruses. These fungi and fungal vectors of disease as well as phytopathogenic bacteria and viruses are for example:

Absidia corymbifera, Alternaria spp, Aphanomyces spp, Ascochyta spp, Aspergillus spp. including A. flavus, A. fumigatus, A. nidulans, A. niger, A. terrus, Aureobasidium spp. including A. pullulans, Blastomyces dermatitidis, Blumeria graminis, Bremia lactucae, Botryosphaeria spp. including B. dothidea, B. obtusa, Botrytis spp. including B. cinerea, Candida spp. including C. albicans, C. glabrata, C. krusei, C. lusitaniae, C. parapsilosis, C. tropicalis, Cephaloascus fragrans, Ceratocystis spp, Cercospora spp. including C. arachidicola. Cercosporidium personatum, Cladosporium spp, Claviceps purpurea, Coccidioides immitis, Cochliobolus spp, Colletotrichum spp. including C. musae, Cryptococcus neoformans. Diaporthe spp, Didymella spp, Drechslera spp, Elsinoe spp. Epldermophyton spp, Erwinia amylovora, Erysiphe spp. including E. cichoracearum, Eutypa lata, Fusarium spp. including F. culmorum, F. graminearum. F. langsethlae. F. moniliforme, F. oxysporum, F. proliferatum, F. subglutinans, F. solani, Gaeumannomyces graminis, Gibberella fujikuroi, Gloeodes pomigena, Gloeosporum musarum, Glomerella cingulate, Guignardia bidwellii, Gymnosporangium juniperi-virginianae, Helminthosporium spp, Hemilela spp, Histoplasma spp. including H. capsulatum, Laetisaria fuciformis, Leptographium lndbergi, Leveillula taurica, Lophodermium seditiosum, Microdochium nivale, Microsporum spp, Monilinla spp, Mucor spp, Mycosphaerella spp. including M. graminicola, M. pomi. Oncobasidium theobromaeon, Ophiostoma piceae, Paracoccidioldes spp. Penicillium spp. including P. digitatum, P. Italicum, Petriellidium spp, Peronosclerospora spp. including P. maydis, P. philippinensis and P. sorghi, Peronospora spp, Phaeosphaeria nodorum, Phakopsora pachyrhizi, Phellinus igniarus, Phialophora spp, Phoma spp. Phomopsis viticola, Phytophthora spp. including P. infestans, Plasmopara spp. including P. halstedii. P. viticola, Pleospora spp. Podosphaera spp. including P. leucotrcha, Polymyxa graminis, Polymyxa betae. Pseudocercosporella herpotricholdes, Pseudomonas spp, Pseudoperonospora spp. including P. cubensis, P. humull, Pseudopeziza tracheiphila, Puccinla Spp. including P. hordei, P. recondita, P. strliformis, P. triticina, Pyrenopeziza spp, Pyrenophora spp, Pyricularia spp. including P. oryzae, Pythium spp. including P. ultimum, Ramularla spp, Rhizoctonia spp, Rhizomucor pusillus, Rhizopus arrhizus, Rhynchosporum spp, Scedosporium spp. including S. apiospermum and S. prolificans, Schizothyrium pomi, Scierotinia spp. Sclerotium spp, Septoria spp, including S. nodorum, S. tritici, Sphaerotheca macularis, Sphaerotheca fusca (Sphaerotheca fuliginea), Sporothorix spp, Stagonospora nodorum, Stemphylium spp. Stereum hirsutum, Thanatephorus cucumeris, Thielaviopsis basicola. Tilletia spp, Trchoderma spp. including T. harzianum, T. pseudokoningii, T. viride, Trchophyton spp, Typhula spp, Uncinula necator, Urocystis spp. Ustilago spp, Venturia spp. including V. inaequalis, Verticillium spp, and Xanthomonas spp.

The compounds of formula (I) may be used for example on turf, ornamentals, such as flowers, shrubs, broad-leaved trees or evergreens, for example conifers, as well as for tree injection, pest management and the like.

Within the scope of present invention, target crops and/or useful plants to be protected typically comprise perennial and annual crops, such as berry plants for example blackberries, blueberries, cranberries, raspberries and strawberries; cereals for example barley, maize (corn), millet, oats, rice, rye, sorghum triticale and wheat; fibre plants for example cotton, flax, hemp, jute and sisal; field crops for example sugar and fodder beet, coffee, hops, mustard, oilseed rape (canola), poppy, sugar cane, sunflower, tea and tobacco; fruit trees for example apple, apricot, avocado, banana, cherry, citrus, nectarine, peach, pear and plum; grasses for example Bermuda grass, bluegrass, bentgrass, centipede grass, fescue, ryegrass, St. Augustine grass and Zoysia grass; herbs such as basil, borage, chives, coriander, lavender, lovage, mint, oregano, parsley, rosemary, sage and thyme; legumes for example beans, lentils, peas and soya beans; nuts for example almond, cashew, ground nut, hazelnut, peanut, pecan, pistachio and walnut; palms for example oil palm; ornamentals for example flowers, shrubs and trees; other trees, for example cacao, coconut, olive and rubber; vegetables for example asparagus, aubergine, broccoli, cabbage, carrot, cucumber, garlic, lettuce, marrow, melon, okra, onion, pepper, potato, pumpkin, rhubarb, spinach and tomato; and vines for example grapes.

The term “useful plants” is to be understood as also including useful plants that have been rendered tolerant to herbicides like bromoxynil or classes of herbicides (such as, for example, HPPD inhibitors, ALS inhibitors, for example primisulfuron, prosuIfuron and trifloxysulfuron, EPSPS (5-enol-pyrovyl-shikimate-3-phosphate-synthase) inhibitors, GS (glutamine synthetase) inhibitors or PPO (protoporphyrinogen-oxidase) inhibitors) as a result of conventional methods of breeding or genetic engineering. An example of a crop that has been rendered tolerant to imidazolinones, e.g. imazamox, by conventional methods of breeding (mutagenesis) is Clearfield® summer rape (Canola). Examples of crops that have been rendered tolerant to herbicides or classes of herbicides by genetic engineering methods include glyphosate- and glufosinate-resistant maize varieties commercially available under the trade names RoundupReady®, Herculex I® and LibertyLink®.

The term “useful plants” is to be understood as also including useful plants which have been so transformed by the use of recombinant DNA techniques that they are capable of synthesising one or more selectively acting toxins, such as are known, for example, from toxin-producing bacteria, especially those of the genus Bacillus.

Examples of such plants are: YieldGard® (maize variety that expresses a CryIA(b) toxin); YieldGard Rootworm® (maize variety that expresses a CryIIIB(b1) toxin); YieldGard Plus® (maize variety that expresses a CryIA(b) and a CryIIIB(b1) toxin); Starlink® (maize variety that expresses a Cry9(c) toxin); Herculex I® (maize variety that expresses a CryIF(a2) toxin and the enzyme phosphinothricine N-acetyitransferase (PAT) to achieve tolerance to the herbicide glufosinate ammonium); NuCOTN 33B® (cotton variety that expresses a CryIA(c) toxin); Bollgard I® (cotton variety that expresses a CryIA(c) toxin); Bollgard II (cotton variety that expresses a CryIA(c) and a CryIIA(b) toxin); VIPCOT® (cotton variety that expresses a VIP toxin); NewLeaf® (potato variety that expresses a CryIIIA toxin); NatureGard® Agrisure® GT Advantage (GA21 glyphosate-tolerant trait), Agrisure® CB Advantage (Bt11 com borer (CB) trait), Agrisure® RW (corn rootworm trait) and Protecta®.

The term “crops” is to be understood as including also crop plants which have been so transformed by the use of recombinant DNA techniques that they are capable of synthesising one or more selectively acting toxins, such as are known, for example, from toxin-producing bacteria, especially those of the genus Bacillus.

Toxins that can be expressed by such transgenic plants include, for example, insecticidal proteins from Bacillus cereus or Bacillus popilliae; or insecticidal proteins from Bacillus thuringiensis, such as 8-endotoxins. e.g. Cry1Ab, Cry1Ac, Cry1F, Cry1Fa2, Cry2Ab, Cry3A, Cry3Bb1 or Cry9C, or vegetative insecticidal proteins (Vip), e.g. Vip1, Vip2, Vip3 or Vip3A; or insecticidal proteins of bacteria colonising nematodes, for example Photorhabdus spp. or Xenorhabdus spp., such as Photorhabdus luminescens, Xenorhabdus nematophilus; toxins produced by animals, such as scorpion toxins, arachnid toxins, wasp toxins and other insect-specific neurotoxins; toxins produced by fungi, such as Streptomycetes toxins, plant lectins, such as pea lectins, barley lectins or snowdrop lectins; agglutinins; proteinase inhibitors, such as trypsin inhibitors, serine protease inhibitors, patatin, cystatin, papain inhibitors; ribosome-inactivating proteins (RIP), such as ricin, maize-RIP, abrin, luffin, saporin or bryodin; steroid metabolism enzymes, such as 3-hydroxysteroldoxidase, ecdysteroid-UDP-glycosyl-transferase, cholesterol oxidases, ecdysone inhibitors, HMG-COA-reductase, ion channel blockers, such as blockers of sodium or calcium channels, juvenile hormone esterase, diuretic hormone receptors, stilbene synthase, bibenzyl synthase, chitinases and glucanases.

Further, in the context of the present invention there are to be understood by 8-endotoxins, for example Cry1Ab, Cry1Ac, Cry1F, Cry1Fa2, Cry2Ab, Cry3A, Cry3Bb1 or Cry9C, or vegetative insecticidal proteins (Vip), for example Vip1, Vip2, Vip3 or Vip3A, expressly also hybrid toxins, truncated toxins and modified toxins. Hybrid toxins are produced recombinantly by a new combination of different domains of those proteins (see, for example, WO 02/15701). Truncated toxins, for example a truncated Cry1Ab, are known. In the case of modified toxins, one or more amino acids of the naturally occurring toxin are replaced. In such amino acid replacements, preferably non-naturally present protease recognition sequences are inserted into the toxin, such as, for example, in the case of Cry3A055, a cathepsin-G-recognition sequence is inserted into a Cry3A toxin (see WO 03/018810).

Examples of such toxins or transgenic plants capable of synthesising such toxins are disclosed, for example, in EP-A-0 374 753, WO93/07278, WO95/34656, EP-A-0 427 529, EP-A-451 878 and WO 03/052073.

The processes for the preparation of such transgenic plants are generally known to the person skilled in the art and are described, for example, in the publications mentioned above. Cry1-type deoxyribonucleic acids and their preparation are known, for example, from WO 95/34656, EP-A-0 367 474, EP-A-0 401 979 and WO 90/13651.

The toxin contained in the transgenic plants imparts to the plants tolerance to harmful insects. Such insects can occur in any taxonomic group of insects, but are especially commonly found in the beetles (Coleoptera), two-winged insects (Diptera) and butterflies (Lepidoptera).

Transgenic plants containing one or more genes that code for an insecticidal resistance and express one or more toxins are known and some of them are commercially available. Examples of such plants are: YieldGard® (maize variety that expresses a Cry1Ab toxin); YieldGard Rootworm® (maize variety that expresses a Cry3Bb1 toxin); YieldGard Plus® (maize variety that expresses a Cry1Ab and a Cry3Bb1 toxin); Starlink® (maize variety that expresses a Cry9C toxin); Herculex I® (maize variety that expresses a Cry1 Fa2 toxin and the enzyme phosphinothricine N-acetytransferase (PAT) to achieve tolerance to the herbicide glufosinate ammonium); NuCOTN 33B® (cotton variety that expresses a Cry1Ac toxin); Boligard I® (cotton variety that expresses a Cry1Ac toxin); Bollgard 116 (cotton variety that expresses a Cry1Ac and a Cry2Ab toxin); VipCot® (cotton variety that expresses a Vip3A and a Cry1Ab toxin); NewLeaf® (potato variety that expresses a Cry3A toxin); NatureGard®, Agrisure® GT Advantage (GA21 glyphosate-tolerant trait), Agrisure® CB Advantage (Bt11 corn borer (CB) trait) and Protecta®.

Further examples of such transgenic crops are:

1. Bt11 Maize from Syngenta Seeds SAS, Chemin de l'Hobit 27, F-31 790 St. Sauveur, France, registration number C/FR/96/05/10. Genetically modified Zea mays which has been rendered resistant to attack by the European com borer (Ostrinia nubilalis and Sesamia nonagrioides) by transgenic expression of a truncated Cry1Ab toxin. Bt11 maize also transgenically expresses the enzyme PAT to achieve tolerance to the herbicide glufosinate ammonium.
2. Bt176 Maize from Syngenta Seeds SAS, Chemin de l'Hobit 27, F-31 790 St. Sauveur, France, registration number C/FR/96/05/10. Genetically modified Zea mays which has been rendered resistant to attack by the European corn borer (Ostrinia nubilalis and Sesamia nonagrioides) by transgenic expression of a Cry1Ab toxin. Bt176 maize also transgenically expresses the enzyme PAT to achieve tolerance to the herbicide glufosinate ammonium.
3. MIR604 Maize from Syngenta Seeds SAS, Chemin de l'Hobit 27, F-31 790 St. Sauveur, France, registration number C/FR/96/05/10. Maize which has been rendered insect-resistant by transgenic expression of a modified Cry3A toxin. This toxin is Cry3A055 modified by insertion of a cathepsin-G-protease recognition sequence. The preparation of such transgenic maize plants is described in WO 03/018810.
4. MON 863 Maize from Monsanto Europe S.A. 270-272 Avenue de Tervuren, B-1150 Brussels, Belgium, registration number C/DE/02/9. MON 863 expresses a Cry3Bb1 toxin and has resistance to certain Coleoptera insects.
5. IPC 531 Cotton from Monsanto Europe S.A. 270-272 Avenue de Tervuren, B-1150 Brussels, Belgium, registration number C/ES/96/02.
6. 1507 Maize from Pioneer Overseas Corporation, Avenue Tedesco, 7 B-1160 Brussels, Belgium, registration number C/NL/00/10. Genetically modified maize for the expression of the protein Cry1F for achieving resistance to certain Lepidoptera insects and of the PAT protein for achieving tolerance to the herbicide glufosinate ammonium.
   7. NK603×MON 810 Maize from Monsanto Europe SA. 270-272 Avenue de Tervuren, B-1150 Brussels, Belgium, registration number C/GB/02/M3/03. Consists of conventionally bred hybrid maize varieties by crossing the genetically modified varieties NK603 and MON 810. NK603×MON 810 Maize transgenically expresses the protein CP4 EPSPS, obtained from Agrobacterium sp. strain CP4, which imparts tolerance to the herbicide Roundup® (contains glyphosate), and also a Cry1Ab toxin obtained from Bacillus thuringiensis subsp. kurstaki which brings about tolerance to certain Lepidoptera, include the European corn borer.

The term “locus” as used herein means fields in or on which plants are growing, or where seeds of cultivated plants are sown, or where seed will be placed into the soil. It includes soil, seeds, and seedlings, as well as established vegetation.

The term “plants” refers to all physical parts of a plant, including seeds, seedlings, saplings, roots, tubers, stems, stalks, foliage, and fruits.

The term “plant propagation material” is understood to denote generative parts of the plant, such as seeds, which can be used for the multiplication of the latter, and vegetative material, such as cuttings or tubers, for example potatoes. There can be mentioned for example seeds (in the strict sense), roots, fruits, tubers, bulbs, rhizomes and parts of plants. Germinated plants and young plants which are to be transplanted after germination or after emergence from the soil, may also be mentioned. These young plants can be protected before transplantation by a total or partial treatment by immersion. Preferably “plant propagation material” is understood to denote seeds.

The compounds of formula I may be used in unmodified form or, preferably, together with the adjuvants conventionally employed in the art of formulation. To this end they may be conveniently formulated in known manner to emulsifiable concentrates, coatable pastes, directly sprayable or dilutable solutions or suspensions, dilute emulsions, wettable powders, soluble powders, dusts, granulates, and also encapsulations e.g. In polymeric substances. As with the type of the compositions, the methods of application, such as spraying, atomising, dusting, scattering, coating or pouring, are chosen in accordance with the intended objectives and the prevailing circumstances. The compositions may also contain further adjuvants such as stabilizers, antifoams, viscosity regulators, binders or tackifiers as well as fertilizers, micronutrient donors or other formulations for obtaining special effects.

Suitable carriers and adjuvants, e.g. for agricultural use, can be solid or liquid and are substances useful in formulation technology, e.g. natural or regenerated mineral substances, solvents, dispersants, wetting agents, tackifiers, thickeners, binders or fertilizers. Such carriers are for example described in WO 97/33890.

Suspension concentrates are aqueous formulations in which finely divided solid particles of the active compound are suspended. Such formulations include anti-settling agents and dispersing agents and may further include a wetting agent to enhance activity as well an anti-foam and a crystal growth inhibitor. In use, these concentrates are diluted in water and normally applied as a spray to the area to be treated. The amount of active ingredient may range from 0.5% to 95% of the concentrate.

Wettable powders are in the form of finely divided particles which disperse readily in water or other liquid carriers. The particles contain the active ingredient retained in a solid matrix. Typical solid matrices include fuller's earth, kaolin clays, silicas and other readily wet organic or inorganic solids. Wettable powders normally contain from 5% to 95% of the active ingredient plus a small amount of wetting, dispersing or emulsifying agent.

Emulsifiable concentrates are homogeneous liquid compositions dispersible in water or other liquid and may consist entirely of the active compound with a liquid or solid emulsifying agent, or may also contain a liquid carrier, such as xylene, heavy aromatic naphthas, isophorone and other non-volatile organic solvents. In use, these concentrates are dispersed in water or other liquid and normally applied as a spray to the area to be treated. The amount of active ingredient may range from 0.5% to 95% of the concentrate.

Granular formulations include both extrudates and relatively coarse particles and are usually applied without dilution to the area in which treatment is required. Typical carriers for granular formulations include sand, fuller's earth, attapulgite clay, bentonite clays, montmorillonite clay, vermiculite, perite, calcium carbonate, brick, pumice, pyrophylite, kaolin, dolomite, plaster, wood flour, ground corn cobs, ground peanut hulls, sugars, sodium chloride, sodium sulphate, sodium silicate, sodium borate, magnesia, mica, iron oxide, zinc oxide, titanium oxide, antimony oxide, cryolite, gypsum, diatomaceous earth, calcium sulphate and other organic or inorganic materials which absorb or which can be coated with the active compound. Granular formulations normally contain 5% to 25% of active ingredients which may include surface-active agents such as heavy aromatic naphthas, kerosene and other petroleum fractions, or vegetable oils; and/or stickers such as dextrins, glue or synthetic resins.

Dusts are free-flowing admixtures of the active ingredient with finely divided solids such as talc, clays, flours and other organic and inorganic solids which act as dispersants and carriers.

Microcapsules are typically droplets or granules of the active ingredient enclosed in an inert porous shell which allows escape of the enclosed material to the surroundings at controlled rates. Encapsulated droplets are typically 1 to 50 microns in diameter. The enclosed liquid typically constitutes 50 to 95% of the weight of the capsule and may include solvent in addition to the active compound. Encapsulated granules are generally porous granules with porous membranes sealing the granule pore openings, retaining the active species in liquid form inside the granule pores. Granules typically range from 1 millimetre to 1 centimetre and preferably 1 to 2 millimetres in diameter. Granules are formed by extrusion, agglomeration or prilling, or are naturally occurring. Examples of such materials are vermiculite, sintered clay, kaolin, attapulgite clay, sawdust and granular carbon. Shell or membrane materials include natural and synthetic rubbers, cellulosic materials, styrene-butadiene copolymers, polyacrylonitriles, polyacrylates, polyesters, polyamides, polyureas, polyurethanes and starch xanthates.

Other useful formulations for agrochemical applications include simple solutions of the active ingredient in a solvent in which it is completely soluble at the desired concentration, such as acetone, alkylated naphthalenes, xylene and other organic solvents. Pressurised sprayers, wherein the active ingredient is dispersed in finely-divided form as a result of vaporisation of a low boiling dispersant solvent carrier, may also be used.

Suitable agricultural adjuvants and carriers that are useful in formulating the compositions of the invention in the formulation types described above are well known to those skilled in the art.

Liquid carriers that can be employed include, for example, water, toluene, xylene, petroleum naphtha, crop oil, acetone, methyl ethyl ketone, cyclohexanone, acetic anhydride, acetonitrile, acetophenone, amyl acetate, 2-butanone, chlorobenzene, cyclohexane, cyclohexanol, alkyl acetates, dlacetonalcohol, 1,2-dichloropropane, diethanolamine, p-diethylbenzene, diethylene glycol, diethylene glycol abietate, diethylene glycol butyl ether, diethylene glycol ethyl ether, diethylene glycol methyl ether, N,N-dimethyl formamide, dimethyl sulfoxide, 1,4-dioxane, dipropylene glycol, dipropylene glycol methyl ether, dipropylene glycol dibenzoate, diproxitol, alkyl pyrrolidinone, ethyl acetate, 2-ethyl hexanol, ethylene carbonate, 1,1,1-trichloroethane, 2-heptanone, alpha pinene, d-limonene, ethylene glycol, ethylene glycol butyl ether, ethylene glycol methyl ether, gamma-butyrolactone, glycerol, glycerol diacetate, glycerol monoacetate, glycerol triacetate, hexadecane, hexylene glycol, isoamyl acetate, isobornyl acetate, isooctane, isophorone, isopropyl benzene, isopropyl myristate, lactic acid, laurylamine, mesityl oxide, methoxy-propanol, methyl isoamyl ketone, methyl isobutyl ketone, methyl laurate, methyl octanoate, methyl oleate, methylene chloride, m-xylene, n-hexane, n-octylamine, odadecanoic acid, octyl amine acetate, oleic acid, oleylamine, o-xylene, phenol, polyethylene glycol (PEG400), propionic acid, propylene glycol, propylene glycol monomethyl ether, p-xylene, toluene, triethyl phosphate, triethylene glycol, xylene sulfonic acid, paraffin, mineral oil, trichloroethylene, perchloroethylene, ethyl acetate, amyl acetate, butyl acetate, methanol, ethanol, isopropanol, and higher molecular weight alcohols such as amyl alcohol, tetrahydrofurfuryl alcohol, hexanol, octanol, etc., ethylene glycol, propylene glycol, glycerine and N-methyl-2-pyrrolidinone. Water is generally the carrier of choice for the dilution of concentrates.

Suitable solid carriers include, for example, talc, titanium dioxide, pyrophyllite clay, silica, attapulgite clay, kieselguhr, chalk, diatomaxeous earth, lime, calcium carbonate, bentonite clay, fuller's earth, cotton seed hulls, wheat flour, soybean flour, pumice, wood flour, walnut shell flour and lignin.

A broad range of surface-active agents are advantageously employed in both said liquid and solid compositions, especially those designed to be diluted with carrier before application. These agents, when used, normally comprise from 0.1% to 15% by weight of the formulation. They can be anionic, cationic, non-ionic or polymeric in character and can be employed as emulsifying agents, wetting agents, suspending agents or for other purposes. Typical surface active agents include salts of alkyl sulfates, such as diethanolammonium lauryl sulphate; alkylarylsulfonate salts, such as calcium dodecylbenzenesulfonate; alkylphenol-alkylene oxide addition products, such as nonylphenol-C.sub. 18 ethoxylate: alcohol-alkylene oxide addition products, such as tridecyl alcohol-C.sub. 16 ethoxylate; soaps, such as sodium stearate; alkylnaphthalenesulfonate salts, such as sodium dibutylnaphthalenesulfonate; dialkyl esters of sulfosuccinate salts, such as sodium di(2-ethylhexyl) sulfosuccinate; sorbitol esters, such as sorbitol oleate; quaternary amines, such as lauryl trimethylammonium chloride; polyethylene glycol esters of fatty acids, such as polyethylene glycol stearate; block copolymers of ethylene oxide and propylene oxide; and salts of mono and dialkyl phosphate esters.

Other adjuvants commonly utilized in agricultural compositions include crystallisation inhibitors, viscosity modifiers, suspending agents, spray droplet modifiers, pigments, antioxidants, foaming agents, anti-foaming agents, light-blocking agents, compatibilizing agents, antifoam agents, sequestering agents, neutralising agents and buffers, corrosion inhibitors, dyes, odorants, spreading agents, penetration aids, micronutrients, emollients, lubricants and sticking agents.

In addition, further, other biocidally active ingredients or compositions may be combined with the compositions of the invention and used in the methods of the invention and applied simultaneously or sequentially with the compositions of the invention. When applied simultaneously, these further active ingredients may be formulated together with the compositions of the invention or mixed in, for example, the spray tank. These further biocidally active ingredients may be fungicides, herbicides, insecticides, bactericides, acaricides, nematicides and/or plant growth regulators.

Pesticidal agents are referred to herein using their common name are known, for example, from “The Pesticide Manual”, 15th Ed., British Crop Protection Council 2009.

In addition, the compositions of the invention may also be applied with one or more systemically acquired resistance inducers (“SAR” inducer). SAR inducers are known and described in, for example. U.S. Pat. No. 6,919,298 and include, for example, salicylates and the commercial SAR inducer acibenzolar-S-methyl.

The compounds of formula (I) are normally used in the form of agrochemical compositions and can be applied to the crop area or plant to be treated, simultaneously or in succession with further compounds. These further compounds can be e.g. fertilizers or micronutrient donors or other preparations, which influence the growth of plants. They can also be selective herbicides or non-selective herbicides as well as insecticides, fungicides, bactericides, nematicides, molluscicides or mixtures of several of these preparations, if desired together with further carriers, surfactants or application promoting adjuvants customarily employed in the art of formulation.

The compounds of formula (I) may be used in the form of (fungicidal) compositions for controlling or protecting against phytopathogenic microorganisms, comprising as active ingredient at least one compound of formula (I) or of at least one preferred individual compound as defined herein, in free form or in agrochemically usable salt form, and at least one of the above-mentioned adjuvants.

The invention therefore provides a composition, preferably a fungicidal composition, comprising at least one compound formula (I) an agriculturally acceptable carrier and optionally an adjuvant. An agricultural acceptable carrier is for example a carrier that is suitable for agricultural use. Agricultural carriers are well known in the art. Preferably said composition may comprise at least one or more pesticidally-active compounds, for example an additional fungicidal active ingredient in addition to the compound of formula (I).

The compound of formula (I) may be the sole active ingredient of a composition or it may be admixed with one or more additional active ingredients such as a pesticide, fungicide, synergist, herbicide or plant growth regulator where appropriate. An additional active ingredient may, in some cases, result in unexpected synergistic activities.

Examples of suitable additional active ingredients include the following: acycloamino acid fungicides, aliphatic nitrogen fungicides, amide fungicides, anilide fungicides, antibiotic fungicides, aromatic fungicides, arsenical fungicides, aryl phenyl ketone fungicides, benzamide fungicides, benzanilide fungicides, benzimidazole fungicides, benzothiazole fungicides, botanical fungicides, bridged diphenyl fungicides, carbamate fungicides, carbanilate fungicides, conazole fungicides, copper fungicides, dicarboximide fungicides, dinitrophenol fungicides, dithiocarbamate fungicides, dithiolane fungicides, furamide fungicides, furanilide fungicides, hydrazide fungicides, imidazole fungicides, mercury fungicides, morpholine fungicides, organophosphorous fungicides, organotin fungicides, oxathin fungicides, oxazole fungicides, phenylsulfamide fungicides, polysulfide fungicides, pyrazole fungicides, pyridine fungicides, pyrimidine fungicides, pyrrole fungicides, quaternary ammonium fungicides, quinoline fungicides, quinone fungicides, quinoxaline fungicides, strobilurin fungicides, sulfonanilide fungicides, thiadiazole fungicides, thiazole fungicides, thiazolidine fungicides, thiocarbamate fungicides, thiophene fungicides, triazine fungicides, triazole fungicides, triazolopyrimidine fungicides, urea fungicides, valinamide fungicides, and zinc fungicides.

Examples of suitable additional active ingredients also include the following: 3-difluoromethyl-1-methyl-1H-pyrazole-4-carboxylic acid (9-dichloromethylene-1,2,3,4-tetrahydro-1,4-methano-naphthalen-5-yl)-amide, 3-difluoromethyl-1-methyl-1H-pyrazole-4-carboxylic acid methoxy-[1-methyl-2-(2,4,6-trichlorophenyl)-ethyl]-amide, 1-methyl-3-difluoromethyl-1H-pyrazole-4-carboxylic acid (2-dichloromethylene-3-ethyl-1-methyl-indan-4-yl)-amide (1072957-71-1), 1-methyl-3-difluoromethyl-1H-pyrazole-4-carboxylic acid (4′-methylsulfanyl-biphenyl-2-yl)-amide, 1-methyl-3-difluoromethyl-4H-pyrazole-4-carboxylic acid [2-(2,4-dichloro-phenyl)-2-methoxy-1-methyl-ethyl]-amide, (5-Chloro-2,4-dimethyl-pyridin-3-yl)-(2,3,4-trimethoxy-6-methyl-phenyl)-methanone, (5-Bromo-4-chloro-2-methoxy-pyridin-3-yl)-(2,3,4-trimethoxy-6-methyl-phenyl)-methanone, 2-{2-[(E)-3-(2,6-Dichloro-phenyl)-1-methyl-prop-2-en-(E)-ylideneaminooxymethyl]-phenyl}-2-[(Z)-methoxyimino]-N-methyl-acetamide, 3-[5-(4-Chloro-phenyl)-2,3-dimethyl-isoxazolidin-3-yl]-pyridine, (E)-N-methyl-2-[2-(2, 5-dimethylphenoxymethyl) phenyl]-2-methoxy-iminoacetamide, 4-bromo-2-cyano-N, N-dimethyl-6-trifluoromethylbenzimidazole-1-sulphonamide, a-[N-(3-chloro-2, 6-xylyl)-2-methoxyacetamido]-y-butyrolactone, 4-chloro-2-cyano-N, -dimethyl-5-p-tolylimidazole-1-sulfonamide, N-allyl-4, 5,-dimethyl-2-trimethylsilylthiophene-3-carboxamide, N—(I-cyano-1, 2-dimethylpropyl)-2-(2, 4-dichlorophenoxy) propionamide, N-(2-methoxy-5-pyridyl)-cyclopropane carboxamide, (.+−.)-cis-1-(4-chlorophenyl)-2-(1H-1,2,4-triazol-1-yl)-cycloheptanol, 2-(1-tert-butyl)-1-(2-chlorophenyl)-3-(1,2,4-triazol-1-yl)-propan-2-ol, 2′,6′-dibromo-2-methyl-4-trifluoromethoxy-4′-trifluoromethyl-1,3-thiazole-5-carboxanilide, 1-imidazolyl-1-(4′-chlorophenoxy)-3,3-dimethylbutan-2-one, methyl (E)-2-[2-[6-(2-cyanophenoxy)pyrimidin-4-yloxy]phenyl]3-methoxyacrylate, methyl (E)-2-[2-[6-(2-thioamidophenoxy)pyrimidin-4-yloxy]phenyl]-3-methoxyacrylate, methyl (E)-2-[2-[6-(2-fluorophenoxy)pyrimidin-4-yloxy]phenyl]-3-methoxyacrylate, methyl (E)-2-[2-[6-(2,6-difluorophenoxy)pyrimidin-4-yloxy]phenyl]-3-methoxyacrylate, methyl (E)-2-[2-[3-(pyrimidin-2-yloxy)phenoxy]phenyl]-3-methoxyacrylate, methyl (E)-2-[2-[3-(5-methylpyridin-2-yloxy)-phenoxy]phenyl]-3-methoxyacrylate, methyl (E)-2-[2-[3-(phenyl-sulphonyloxy)phenoxy]phenyl-3-methoxyacrylate, methyl (E)-2-[2-[3-(4-nitrophenoxy)phenoxy]phenyl]-3-methoxyacrylate, methyl (E)-2-[2-phenoxyphenyl]-3-methoxyacrylate, methyl (E)-2-[2-(3,5-dimethyl-benzoyl)pyrrol-1-yl]-3-methoxyacrylate, methyl (E)-2-[2-(3-methoxyphenoxy)phenyl]-3-methoxyacrylate, methyl (E)-2[2-(2-phenylethen-1-yl)-phenyl]-3-methoxyacrylate, methyl (E)-2-[2-(3,5-dichlorophenoxy)pyridin-3-yl]-3-methoxyacrylate, methyl (E)-2-(2-(3-(1,1,2,2-tetrafluoroethoxy)phenoxy)phenyl)-3-methoxyacrylate, methyl (E)-2-(2-[3-(alpha-hydroxybenzyl)phenoxy]phenyl)-3-methoxyacrylate, methyl (E)-2-(2-(4-phenoxypyridin-2-yloxy)phenyl)-3-methoxyacrylate, methyl (E)-2-[2-(3-n-propyloxy-phenoxy)phenyl]3-methoxyacrylate, methyl (E)-2-[2-(3-isopropyloxyphenoxy)phenyl]-3-methoxyacrylate, methyl (E)-2-[2-[3-(2-fluorophenoxy)phenoxy]phenyl]-3-methoxyacrylate, methyl (E)-2-[2-(3-ethoxyphenoxy)phenyl]-3-methoxyacrylate, methyl (E)-2-[2-(4-tert-butyl-pyridin-2-yloxy)phenyl]-3-methoxyacrylate, methyl (E)-2-[2-[3-(3-cyanophenoxy)phenoxy]phenyl]-3-methoxyacrylate, methyl (E)-2-[2-[(3-methyl-pyridin-2-yloxymethyl)phenyl]-3-methoxyacrylate, methyl (E)-2-[2-[6-(2-methyl-phenoxy)pyrimidin-4-yloxy]phenyl]-3-methoxyacrylate, methyl (E)-2-[2-(5-bromo-pyridin-2-yloxymethyl)phenyl)-3-methoxyacrylate, methyl (E)-2-[2-(3-(3-iodopyridin-2-yloxy)phenoxy)phenyl-3-methoxyacrylate, methyl (E)-2-[2-[6-(2-chloropyridin-3-yloxy)pyrimidin-4-yloxy]phenyl]-3-methoxyacrylate, methyl (E),(E)-2-[2-(5,6-dimethylpyrazin-2-ylmethyloximinomethyl)phenyl]-3-methoxyacrylate, methyl (E)-2-{2-[6-(6-methylpyridin-2-yloxy)pyrimidin-4-yloxy]phenyl}-3-methoxy-acrylate, methyl (E),(E)-2-{2-(3-methoxyphenyl)methyloximinomethyl]-phenyl}-3-methoxyacrylate, methyl (E)-2-{2-(6-(2-azidophenoxy)-pyrimidin-4-yloxy]phenyl}-3-methoxyacrylate, methyl (E),(E)-2-{2-[6-phenylpyrimidin-4-yl)-methyloximinomethyl]phenyl}-3-methoxyacrylate, methyl (E),(E)-2-{2-[(4-chlorophenyl)-methyloximinomethyl]-phenyl}-3-methoxyacrylate, methyl (E)-2-{2-[6-(2-n-propylphenoxy)-1,3,5-triazin-4-yloxy]phenyl}-3-methoxyacrylate, methyl (E),(E)-2-{2-[(3-nitrophenyl)methyloximinomethyl]phenyl}-3-methoxyacrylate, 3-chloro-7-(2-aza-2,7,7-trimethyl-oct-3-en-5-ine), 2,6-dichloro-N-(4-trifluoromethylbenzyl)-benzamide, 3-iodo-2-propinyl alcohol, 4-chlorophenyl-3-iodopropargyl formal, 3-bromo-2,3-diiodo-2-propenyl ethylcarbamate, 2,3,3-triiodoallyl alcohol, 3-bromo-2,3-diiodo-2-propenyl alcohol, 3-iodo-2-propinyl n-butylcarbamate, 3-iodo-2-propinyl n-hexylcarbamate, 3-iodo-2-propinyl cyclohexyl-carbamate, 3-iodo-2-propinyl phenylcarbamate; phenol derivatives, such as tribromophenol, tetrachlorophenol, 3-methyl-4-chlorophenol, 3,5-dimethyl-4-chlorophenol, phenoxyethanol, dichlorophene, o-phenylphenol, m-phenylphenol, p-phenylphenol, 2-benzyl-4-chlorophenol, 5-hydroxy-2(5H)-furanone; 4,5-dichlorodithiazolinone, 4,5-benzodithiazolinone, 4,5-trimethylenedithiazolinone, 4,5-dichloro-(3H)-1,2-dithiol-3-one, 3,5-dimethyl-tetrahydro-1,3,5-thiadiazine-2-thione, N-(2-p-chlorobenzoylethyl)-hexaminium chloride, acibenzolar, acypetacs, alanycarb, albendazole, aldimorph, allicin, allyl alcohol, ametoctradin, amisulbrom, amobam, ampropytfos, anilazine, asomate, aureofungin, azaconazole, azafendin, azithiram, azoxystrobin, barium polysulfide, benalaxyl, benalaxyl-M, benodanil, benomyl, benquinox, bentaluron, benthiavalicarb, benthiazole, benzalkonium chloride, benzamacril, benzamorf, benzohydroxamic acid, benzovindiflupyr, berberine, bethoxazin, biloxazol, binapacryl, biphenyl, bitertanol, bithionol, bixafen, blasticidin-S, boscalid, bromothalonil, bromuconazole, bupirimate, buthiobate, butylamine calcium polysulfide, captafol, captan, carbamorph, carbendazim, carbendazim chiorhydrate, carboxin, carpropamid, carvone, CGA41396, CGA41397, chinomethionate, chitosan, chlobenthiazone, chloraniformethan, chloranil, chlorfenazole, chloroneb, chloropicrin, chlorothalonil, chlorozolinate, chlozolinate, climbazole, clotrimazole, clozylacon, copper containing compounds such as copper acetate, copper carbonate, copper hydroxide, copper naphthenate, copper oleate, copper oxychloride, copper oxyquinolate, copper silicate, copper sulphate, copper tallate, copper zinc chromate and Bordeaux mixture, cresol, cufraneb, cuprobam, cuprous oxide, cyazofamid, cyclafuramid, cycloheximide, cyflufenamid, cymoxanil, cypendazole, cyproconazole, cyprodinil, dazomet, debacarb, decafentin, dehydroacetic acid, di-2-pyridyl disulphide 1, 1′-dioxide, dichlofluanid, diciomezine, dichlone, dicloran, dichiorophen, dichlozoline, diclobutrazol, dicocymet, diethofencarb, difenoconazole, difenzoquat, diflumetorim, O. O-di-iso-propyl-S-benzyl thiophosphate, dimefluazole, dimetachlone, dimetconazole, dimethomorph, dimethirimol, diniconazole, diniconazole-M, dinobuton, dinocap, dinocton, dinopenton, dinosulfon, dinoterbon, diphenylamine, dipyrithione, disulfiram, ditalimfos, dithianon, dithioether, dodecyl dimethyl ammonium chloride, dodemorph, dodicin, dodine, doguadine, drazoxolon, edifenphos, enestroburin, epoxiconazole, etaconazole, etem, ethaboxam, ethirimol, ethoxyquin, ethilicin, ethyl (Z)—N-benzyl-N([methyl (methyl-thioethylideneamino-oxycarbonyl) amino] thio)-β-alaninate, etridiazole, famoxadone, fenamidone, fenaminosulf, fenapanil, fenarimol, fenbuconazole, fenfuram, fenhexamid, fenitropan, fenoxanil, fenpicionil, fenpicoxamid, fenpropidin, fenpropimorph, fenpyrazamine, fentin acetate, fentin hydroxide, ferbam, ferimzone, fluazinam, fludioxoni, flumetover, flumorph, flupicolide, fluopyram, fluorolmide, fluotrimazole, fluoxastrobin, fluquinconazole, flusilazole, flusulfamide, flutanil, flutolanil, flutriafol, fluxapyroxad, folpet, formaldehyde, fosetyl, fuberidazole, furalaxyl, furametpyr, furcarbanil, furconazole, furfural, furmecyclox, furophanate, glyodln, griseofuMn, guazatine, halacrlnate, hexa chlorobenzene, hexachlorobutadlene, hexachlorophene, hexaconazole, hexylhiofos, hydrargaphen, hydroxyisoxazole, hymexazole, imazalil, imazalil sulphate, imibenconazole, iminoctadine, iminoctadine triacetate, inezin, iodocarb, ipconazole, ipfentrifluconazole, iprobenfos, iprodione, iprovalicarb, isopropanyl butyl carbamate, isoprothiolane, isopyrazam, isotianil, isovaledione, izopamfos, kasugamycin, kresoxim-methyl. LY186054, LY211795, LY248908, mancozeb, mandipropamid, maneb, mebenil, mecarbinzid, mefenoxam, mefentrifluconazole, mepanipyrim, mepronil, mercuric chloride, mercurous chloride, meptyldinocap, metalaxyl, metalaxyl-M, metam, metazoxolon, metconazole, methasulfocarb, methfuroxam, methyl bromide, methyl iodide, methyl isothiocyanate, metiram, metiram-zinc, metominostrobin, metrafenone, metsulfovax, milneb, moroxydine, myclobutani, myclozolin, nabam, natamycin, neoasozin, nickel dimethyidithiocarbamate, nitrostyrene, nitrothal-iso-propyl, nuarimol, ochilnone, ofurace, organomercury compounds, orysastrobin, osthol, oxadixyl, oxasulfuron, oxine-copper, oxolinic acid, oxpoconazole, oxycarboxin, parinol, pefurazoate, penconazole, pencycuron, penflufen, pentachlorophenol, penthiopyrad, phenamacril, phenazin oxide, phosdiphen, phosetyl-AI, phosphorus acids, phthalide, picoxystrobin, piperalin, polycarbamate, polyoxin D, polyoxrim, polyram, probenazole, prochloraz, procymidone, propamidine, propamocarb, propiconazole, propineb, propionic acid, proquinazid, prothiocarb, prothioconazole, pydiflumetofen, pyracarbolid, pyraciostrobin, pyrametrostrobin, pyraoxystrobin, pyrazophos, pyribencarb, pyridinitril, pyrifenox, pyrimethanil, pyriofenone, pyroquilon, pyroxychlor, pyroxyfur, pyrrolnitrin, quatemary ammonium compounds, quinacetol, quinazamid, quinconazole, quinomethionate, quinoxyfen, quintozene, rabenzazole, santonin, sedaxane, slthiofam, simeconazole, sipconazole, sodium pentachlorophenate, solatenol, spiroxamine, streptomycin, sulphur, sultropen, tebuconazole, tebfloquin, tecioftalam, tecnazene, tecoram, tetraconazole, thiabendazole, thiadifluor, thicyofen, thifluzamide, 2-(thiocyanomethytthio) benzothiazole, thiophanate-methyl, thioquinox, thiram, tiadinil, timibenconazole, tioxymid, tolciofos-methyl, tolylfluanid, triadimefon, triadimenol, triamiphos, triarimol, triazbutil, triazoxide, tricyclazole, tridemorph, trifloxystrobin, triflumazole, triforine, triflumizole, triticonazole, uniconazole, urbacide, validamycin, valifenalate, vapam, vinclozolin, zarilamid, zineb, ziram, and zoxamide.

The compounds of the invention may also be used in combination with anthelmintic agents. Such anthelmintic agents include, compounds selected from the macrocyclic lactone class of compounds such as Ivermectin, avermectin, abamectin, emamectin, eprinomectin, doramectin, selamectin, moxidectin, nemadectin and milbemycin derivatives as described in EP-357460, EP-444964 and EP-594291. Additional anthelmintic agents include semisynthetic and biosynthetic avermectin/milbemycin derivatives such as those described in U.S. Pat. No. 5,015,630, WO-9415944 and WO-9522552. Additional anthelmintic agents include the benzimidazoles such as albendazole, cambendazole, fenbendazole, flubendazole, mebendazole, oxfendazole, oxibendazole, parbendazole, and other members of the class. Additional anthelmintic agents include imidazothiazoles and tetrahydropyrimidines such as tetramisole, levamisole, pyrantel pamoate, oxantel or morantel. Additional anthelmintic agents include flukicides, such as triclabendazole and clorsulon and the cestocides, such as praziquantel and epsiprantel.

The compounds of the invention may be used in combination with derivatives and analogues of the paraherquamide/marcfortine class of anthelmintic agents, as well as the antiparasitic oxazolines such as those disclosed in U.S. Pat. Nos. 5,478,855, 4,639,771 and DE-19520936.

The compounds of the invention may be used in combination with derivatives and analogues of the general class of dioxomorpholine antiparasitic agents as described in WO 96/15121 and also with anthelmintic active cyclic depsipeptides such as those described in WO 96/11945, WO 93/19053, WO 93/25543, EP 0 626 375, EP 0 382 173, WO 94/19334, EP 0 382 173, and EP 0 503 538.

The compounds of the invention may be used in combination with other ectoparasiticides; for example, fipronil; pyrethroids; organophosphates; insect growth regulators such as lufenuron; ecdysone agonists such as tebufenozide and the like; neonicotinoids such as imidacloprid and the like.

The compounds of the invention may be used in combination with terpene alkaloids, for example those described in International Patent Application Publication Numbers WO 95/19363 or WO 04/72086, particularly the compounds disclosed therein.

Other examples of such biologically active compounds that the compounds of the invention may be used in combination with include but are not restricted to the following: Organophosphates: acephate, azamethiphos, azinphos-ethyl, azinphos-methyl, bromophos, bromophos-ethyl, cadusafos, chiorethoxyphos, chlorpyrifos, chlorfenvinphos, chlormephos, demeton, demeton-S-methyl, demeton-S-methyl sulphone, dialifos, diazinon, dichlorvos, dicrotophos, dimethoate, disulfoton, ethion, ethoprophos, etrimfos, famphur, fenamiphos, fenitrothion, fensulfothion, fenthion, flupyrazofos, fonofos, formothion, fosthiazate, heptenophos, isazophos, isothioate, soxathion, malathion, methacriphos, methamidophos, methidathion, methyl-parathion, mevinphos, monocrotophos, naled, omethoate, oxydemeton-methyl, paraoxon, parathion, parathion-methyl, phenthoate, phosalone, phosfolan, phosphocarb, phosmet, phosphamidon, phorate, phoxim, pirimiphos, pirimiphos-methyl, profenofos, propaphos, proetamphos, prothiofos, pyraciofos, pyridapenthion, quinalphos, sulprophos, temephos, terbufos, tebupirimfos, tetrachlorvinphos, thimeton, triazophos, trichlorfon, vamidothion.

Carbamates: alanycarb, aldicarb, 2-sec-butylphenyl methylcarbamate, benfuracarb, carbaryl, carbofuran, carbosulfan, cloethocarb, ethiofencarb, fenoxycarb, fenthiocarb, furathiocarb, HCN-801, isoprocarb, indoxacarb, methiocarb, methomyl, 5-methyl-m-cumenylbutyryl(methyl)carbamate, oxamyl, pirimicarb, propoxur, thiodicarb, thiofanox, triazamate, UC-51717.

Pyrethroids: acrinathin, allethrin, alphametrin, 5-benzyl-3-furylmethyl (E)-(1R)-cis-2,2-dimethyl-3-(2-oxothiolan-3-ylidenemethyl)cyclopropanecarboxylate, bifenthrin, beta-cyfluthrin, cyfluthrin, a-cypermethrin, beta-cypermethrin, bioallethrin, bioallethrin((S)-cyclopentylisomer), bioresmethrin, bifenthrin, NCI-85193, cycloprothrin, cyhalothrin, cythithrin, cyphenothrin, deltamethrin, empenthrin, esfenvalerate, ethofenprox, fenfluthrin, fenpropathrin, fenvalerate, flucythrinate, flumethrin, fluvalinate (D isomer), imiprothrin, cyhalothrin, lambda-cyhalothrin, permethrin, phenothrin, prallethrin, pyrethrins (natural products), resmethrin, tetramethrin, transfluthrin, theta-cypermethrin, silafluofen, t-fluvalinate, tefluthrin, tralomethrin, Zeta-cypermethrin.

Arthropod growth regulators: a) chitin synthesis inhibitors: benzoylureas: chlorfluazuron, diflubenzuron, fluazuron, flucycloxuron, flufenoxuron, hexaflumuron, lufenuron, novaluron, teflubenzuron, triflumuron, buprofezin, diofenolan, hexythiazox, etoxazole, chlorfentazine; b) ecdysone antagonists: halofenozide, methoxyfenozide, tebufenozide; c) juvenoids: pyriproxyfen, methoprene (including S-methoprene), fenoxycarb; d) lipid biosynthesis inhibitors: spirodiclofen.

Other antiparasitics: acequinocyl, amitraz, AKD-1022, ANS-118, azadirachtin, Bacillus thuringiensis, bensultap, bifenazate, binapacryl, bromopropylate, BTG-504, BTG-505, camphechlor, cartap, chlorobenzilate, chlordimeform, chlorfenapyr, chromafenozide, clothianidine, cyromazine, diacloden, diafenthiuron, DBI-3204, dinactin, dihydroxymethyldihydroxypyrrolidine, dinobuton, dinocap, endosulfan, ethiprole, ethofenprox, fenazaquin, flumite, MTI-800, fenpyroximate, fluacrypyrim, flubenzimine, flubrocythrinate, flufenzine, flufenprox, fluproxyfen, halofenprox, hydramethylnon, IKI-220, kanemite, NC-196, neem guard, nidinorterfuran, nitenpyram, SD-35651, WL-108477, pirydaryl, propargite, protrifenbute, pymethrozine, pyridaben, pyrimidifen, NC-111, R-195, RH-0345, RH-2485, RYI-210, S-1283, S-1833, SI-8601, silafluofen, silomadine, spinosad, tebufenpyrad, tetradifon, tetranadin, thiacloprid, thiocyclam, thiamethoxam, tolfenpyrad, triazamate, triethoxyspinosyn, trinactin, verbutin, vertalec, YI-5301.

Biological agents: Bacillus thuringiensis ssp aizawai, kurstaki, Bacillus thuringiensis delta endotoxin, baculovirus, entomopathogenic bacteria, virus and fungi.

Bactericides: chlortetracycline, oxytetracycline, streptomycin.

Other biological agents: enrofloxacin, febantel, penethamate, moloxicam, cefalexin, kanamycin, pimobendan, clenbuterol, omeprazole, tiamulin, benazepril, pyriprole, cefquinome, florfenicol, buserelin, cefovecin, tulathromycin, ceftiour, carprofen, metaflumizone, praziquarantel, triclabendazole.

The following mixtures of the compounds of formula (I) with active ingredients are preferred.

The abbreviation “TX” means one compound selected from the group consisting of the compounds described in Tables 1.1 to 1.19 (below) or Table T1 (below).

an adjuvant selected from the group of substances consisting of petroleum oils (628)+TX,

an acaricide selected from the group of substances consisting of 1,1-bis(4-chlorophenyl)-2-ethoxyethanol (IUPAC name) (910)+TX, 2,4-dichlorophenyl benzenesulfonate (IUPAC/Chemical Abstracts name) (1059)+TX, 2-fluoro-N-methyl-N-1-naphthylacetamide (IUPAC name) (1295)+TX, 4-chiorophenyl phenyl sulfone (IUPAC name) (981)+TX, abamectin (1)+TX, acequinocyl (3)+TX, acetoprole [CCN]+TX, acrinathrin (9)+TX, aldicarb (16)+TX, aldoxycarb (863)+TX, alpha-cypermethrin (202)+TX, amidithion (870)+TX, amidoflumet [CCN]+TX, amidothioate (872)+TX, amiton (875)+TX, amiton hydrogen oxalate (875)+TX, amitraz (24)+TX, aramite (881)+TX, arsenous oxide (882)+TX, AVI 382 (compound code)+TX, AZ 60541 (compound code)+TX, azinphos-ethyl (44)+TX, azinphos-methyl (45)+TX, azobenzene (IUPAC name) (888)+TX, azocyclotin (46)+TX, azothoate (889)+TX, benomyl (62)+TX, benoxafos [CCN]+TX, benzoximate (71)+TX, benzyl benzoate (IUPAC name) [CCN]+TX, bifenazate (74)+TX, bifenthrin (76)+TX, binapacryl (907)+TX, brofenvalerate+TX, bromocyclen (918)+TX, bromophos (920)+TX, bromophos-ethyl (921)+TX, bromopropylate (94)+TX, buprofezin (99)+TX, butocarboxim (103)+TX, butoxycarboxim (104)+TX, butylpyridaben+TX, calcium polysulfide (IUPAC name) (111)+TX, camphechlor (941)+TX, carbanolate (943)+TX, carbaryl (115)+TX, carbofuran (118)+TX, carbophenothion (947)+TX, CGA 50′439 (development code) (125)+TX, chinomethionat (126)+TX, chlorbenside (959)+TX, chlordimeform (964)+TX, chlordimeform hydrochloride (964)+TX, chlorfenapyr (130)+TX, chlorfenethol (968)+TX, chlorfenson (970)+TX, chlorfensulfide (971)+TX, chlorfenvinphos (131)+TX, chlorobenzilate (975)+TX, chloromebuform (977)+TX, chloromethiuron (978)+TX, chloropropylate (983)+TX, chlorpyrifos (145)+TX, chlorpyrifos-methyl (146)+TX, chlorthiophos (994)+TX, cinerin I (696)+TX, cinerin II (696)+TX, cinerins (696)+TX, clofentezine (158)+TX, closantel [CCN]+TX, coumaphos (174)+TX, crotamiton [CCN]+TX, crotoxyphos (1010)+TX, cufraneb (1013)+TX, cyanthoate (1020)+TX, cyflumetofen (CAS Reg. No.: 400882-07-7)+TX, cyhalothrin (196)+TX, cyhexatin (199)+TX, cypermethrin (201)+TX, DCPM (1032)+TX, DDT (219)+TX, demephion (1037)+TX, demephlon-O (1037)+TX, demephion-S (1037)+TX, demeton (1038)+TX, demeton-methyl (224)+TX, demeton-O (1038)+TX, demeton-O-methyl (224)+TX, demeton-S (1038)+TX, demeton-S-methyl (224)+TX, demeton-S-methylsulfon (1039)+TX, diafenthiuron (226)+TX, dialifos (1042)+TX, diazinon (227)+TX, dichlofluanid (230)+TX, dichlorvos (236)+TX, dicliphos+TX, dicofol (242)+TX, dicrotophos (243)+TX, dienochlor (1071)+TX, dimefox (1081)+TX, dimethoate (262)+TX, dinactin (653)+TX, dinex (1089)+TX, dinex-diclexine (1089)+TX, dinobuton (269)+TX, dinocap (270)+TX, dinocap-4 [CCN]+TX, dinocap-6 [CCN]+TX, dinocton (1090)+TX, dinopenton (1092)+TX, dinosulfon (1097)+TX, dinoterbon (1098)+TX, dioxathion (1102)+TX, diphenyl sulfone (IUPAC name) (1103)+TX, disulfiram [CCN]+TX, disulfoton (278)+TX, DNOC (282)+TX, dofenapyn (1113)+TX, doramectin [CCN]+TX, endosulfan (294)+TX, endothion (1121)+TX, EPN (297)+TX, eprinomectin [CCN]+TX, ethion (309)+TX, ethoate-methyl (1134)+TX, etoxazole (320)+TX, etrimfos (1142)+TX, fenazaflor (1147)+TX, fenazaquin (328)+TX, fenbutatin oxide (330)+TX, fenothiocarb (337)+TX, fenpropathrin (342)+TX, fenpyrad+TX, fenpyroximate (345)+TX, fenson (1157)+TX, fentrifanil (1161)+TX, fenvalerate (349)+TX, fipronil (354)+TX, fluacrypyrim (360)+TX, fluazuron (1166)+TX, flubenzimine (1167)+TX, flucycloxuron (366)+TX, flucythrinate (367)+TX, fluenetil (1169)+TX, flufenoxuron (370)+TX, flumethrin (372)+TX, fluorbenside (1174)+TX, fluvalinate (1184)+TX, FMC 1137 (development code) (1185)+TX, formetanate (405)+TX, formetanate hydrochloride (405)+TX, formothion (1192)+TX, formparanate (1193)+TX, gamma-HCH (430)+TX, glyodin (1205)+TX, halfenprox (424)+TX, heptenophos (432)+TX, hexadecyl cyclopropanecarboxylate (IUPAC/Chemical Abstracts name) (1216)+TX, hexythiazox (441)+TX, iodomethane (IUPAC name) (542)+TX, isocarbophos (473)+TX, isopropyl O-(methoxyaminothiophosphoryl)salicylate (IUPAC name) (473)+TX, ivermectin [CCN]+TX, jasmolin I (696)+TX, jasmolin 11 (696)+TX, jodfenphos (1248)+TX, lindane (430)+TX, lufenuron (490)+TX, malathion (492)+TX, malonoben (1254)+TX, mecarbam (502)+TX, mephosfolan (1261)+TX, mesulfen [CCN]+TX, methacrifos (1266)+TX, methamidophos (527)+TX, methidathion (529)+TX, methiocarb (530)+TX, methomyl (531)+TX, methyl bromide (537)+TX, metolcarb (550)+TX, mevinphos (556)+TX, mexacarbate (1290)+TX, milbemectin (557)+TX, milbemycin oxime [CCN]+TX, mipafox (1293)+TX, monocrotophos (561)+TX, morphothion (1300)+TX, moxidectin [CCN]+TX, naled (567)+TX, NC-184 (compound code)+TX, NC-512 (compound code)+TX, nifluridide (1309)+TX, nikkomycins [CCN]+TX, nitrilacarb (1313)+TX, nitrilacarb 1:1 zinc chloride complex (1313)+TX, NNI-0101 (compound code)+TX, NNI-0250 (compound code)+TX, omethoate (594)+TX, oxamyl (602)+TX, oxydeprofos (1324)+TX, oxydisulfoton (1325)+TX, pp′-DDT (219)+TX, parathion (615)+TX, permethrin (626)+TX, petroleum oils (628)+TX, phenkapton (1330)+TX, phenthoate (631)+TX, phorate (636)+TX, phosalone (637)+TX, phosfolan (1338)+TX, phosmet (638)+TX, phosphamidon (639)+TX, phoxim (642)+TX, pirimiphos-methyl (652)+TX, polychloroterpenes (traditional name) (1347)+TX, polynactins (653)+TX, proclonol (1350)+TX, profenofos (662)+TX, promacyl (1354)+TX, propargite (671)+TX, propetamphos (673)+TX, propoxur (678)+TX, prothidathion (1360)+TX, prothoate (1362)+TX, pyrethrin 1 (696)+TX, pyrethrin II (696)+TX, pyrethrins (696)+TX, pyridaben (699)+TX, pyridaphenthion (701)+TX, pyrimidifen (706)+TX, pyrimitate (1370)+TX, quinalphos (711)+TX, quintiofos (1381)+TX, R-1492 (development code) (1382)+TX, RA-17 (development code) (1383)+TX, rotenone (722)+TX, schradan (1389)+TX, sebufos+TX, selamectin [CCN]+TX, SI-0009 (compound code)+TX, sophamide (1402)+TX, spirodiclofen (738)+TX, spiromesifen (739)+TX, SSI-121 (development code) (1404)+TX, sulfiram [CCN]+TX, sulfluramid (750)+TX, sulfotep (753)+TX, sulfur (754)+TX, SZI-121 (development code) (757)+TX, tau-fluvalinate (398)+TX, tebufenpyrad (763)+TX, TEPP (1417)+TX, terbam+TX, tetrachlorvinphos (777)+TX, tetradifon (786)+TX, tetranactin (653)+TX, tetrasul (1425)+TX, thiafenox+TX, thlocarboxime (1431)+TX, thiofanox (800)+TX, thiometon (801)+TX, thioquinox (1436)+TX, thuringlensin [CCN]+TX, triamiphos (1441)+TX, triarathene (1443)+TX, triazophos (820)+TX, triazuron+TX, trichlorfon (824)+TX, trifenofos (1455)+TX, trinactin (653)+TX, vamidothion (847)+TX, vaniliprole [CCN] and YI-5302 (compound code)+TX,

an algicide selected from the group of substances consisting of bethoxazin [CCN]+TX, copper dioctanoate (IUPAC name) (170)+TX, copper sulfate (172)+TX, cybutryne [CCN]+TX, dichlone (1052)+TX, dichlorophen (232)+TX, endothal (295)+TX, fentin (347)+TX, hydrated lime [CCN]+TX, nabam (566)+TX, quinoclamine (714)+TX, quinonamid (1379)+TX, simazine (730)+TX, triphenyltin acetate (IUPAC name) (347) and triphenyltin hydroxide (IUPAC name) (347)+TX,

an anthelmintic selected from the group of substances consisting of abamectin (1)+TX, crufomate (1011)+TX, doramectin [CCN]+TX, emamectin (291)+TX, emamectin benzoate (291)+TX, eprinomectin [CCN]+TX, ivermectin [CCN]+TX, milbemycin oxime [CCN]+TX, moxidectin [CCN]+TX, piperazine [CCN]+TX, selamectin [CCN]+TX, spinosad (737) and thiophanate (1435)+TX,

an avicide selected from the group of substances consisting of chloralose (127)+TX, endrin (1122)+TX, fenthion (346)+TX, pyridin-4-amine (IUPAC name) (23) and strychnine (745)+TX, a bactericide selected from the group of substances consisting of 1-hydroxy-1H-pyridine-2-thione (IUPAC name) (1222)+TX, 4-(quinoxalin-2-ylamino)benzenesulfonamide (IUPAC name) (748)+TX, 8-hydroxyquinoline sulfate (446)+TX, bronopol (97)+TX, copper dioctanoate (IUPAC name) (170)+TX, copper hydroxide (IUPAC name) (169)+TX, cresol [CCN]+TX, dichlorophen (232)+TX, dipyrithione (1105)+TX, dodicin (1112)+TX, fenaminosulf (1144)+TX, formaldehyde (404)+TX, hydrargaphen [CCN]+TX, kasugamycin (483)+TX, kasugamycin hydrochloride hydrate (483)+TX, nickel bis(dimethyldithiocarbamate) (IUPAC name) (1308)+TX, nitrapyrin (580)+TX, octhilinone (590)+TX, oxolinic acid (606)+TX, oxytetracycline (611)+TX, potassium hydroxyquinoline sulfate (446)+TX, probenazole (658)+TX, streptomycin (744)+TX, streptomycin sesquisulfate (744)+TX, tecloftalam (766)+TX, and thiomersal [CCN]+TX,

a biological agent selected from the group of substances consisting of Adoxophyes orana GV (12)+TX, Agrobacterium radiobacter (13)+TX, Amblyseius spp. (19)+TX, Anagrapha falcifera NPV (28)+TX, Anagrus atomus (29)+TX, Aphelinus abdominalis (33)+TX, Aphidius colemani (34)+TX, Aphidoletes aphidimyza (35)+TX, Autographa californica NPV (38)+TX, Bacillus firmus (48)+TX, Bacillus sphaericus Nekie (scientific name) (49)+TX, Bacillus thuringiensis Berliner (scientific name) (51)+TX, Bacillus thuringiensis subsp. aizawai (scientific name) (51)+TX, Bacillus thuringiensis subsp. israelensis (scientific name) (51)+TX, Bacillus thuringiensis subsp. japonensis (scientific name) (51)+TX, Bacillus thuringiensis subsp. kurstaki (scientific name) (51)+TX, Bacillus thuringiensis subsp. tenebrionis (scientific name) (51)+TX, Beauveria bassiana (53)+TX, Beauveria bongniartii (54)+TX, Chrysoperla carnea (151)+TX, Cryptolaemus montrouzieni (178)+TX, Cydia pomonella GV (191)+TX, Dacnusa sibirica (212)+TX, Diglyphus isaea (254)+TX, Encarsia formosa (scientific name) (293)+TX, Eretmocerus eremicus (300)+TX, Helicoverpa zea NPV (431)+TX, Heterorhabditis bacteriophora and H. megidis (433)+TX, Hippodamia convergens (442)+TX, Leptomastix dactylopil (488)+TX, Macrolophus caliginosus (491)+TX, Mamestra brassicae NPV (494)+TX, Metaphycus helvolus (522)+TX, Metarhizium anisopliae var. acridum (scientific name) (523)+TX, Metarhizium anisopliae var. anisopliae (scientific name) (523)+TX, Neodiprion sertifer NPV and N. lecontei NPV (575)+TX, Orius spp. (596)+TX, Paecilomyces fumosoroseus (613)+TX, Phytoseiulus persimilis (644)+TX, Spodoptera exigua multicapsid nuclear polyhedrosis virus (scientific name) (741)+TX, Steinernema bibionis (742)+TX, Steinernema carpocapsae (742)+TX, Steinernema feltiae (742)+TX, Steinernema glaseri (742)+TX, Steinernema riobrave (742)+TX, Steinernema riobravis (742)+TX, Steinernema scapterisci (742)+TX, Steinernema spp. (742)+TX, Trichogramma spp. (826)+TX, Typhlodromus occidentalis (844) and Verticillum lecanii (848)+TX,

a soil sterilant selected from the group of substances consisting of iodomethane (IUPAC name) (542) and methyl bromide (537)+TX,

a chemosterilant selected from the group of substances consisting of apholate [CCN]+TX, bisazir [CCN]+TX, busulfan [CCN]+TX, diflubenzuron (250)+TX, dimatif [CCN]+TX, hemel [CCN]+TX, hempa [CCN]+TX, metepa [CCN]+TX, methiotepa [CCN]+TX, methyl apholate [CCN]+TX, morzid [CCN]+TX, penfluron [CCN]+TX, tepa [CCN]+TX, thiohempa [CCN]+TX, thiotepa [CCN]+TX, tretamine [CCN] and uredepa [CCN]+TX,

an insect pheromone selected from the group of substances consisting of (E)-dec-5-en-1-yl acetate with (E)-dec-5-en-1-ol (IUPAC name) (222)+TX, (E)-tridec-4-en-1-yl acetate (IUPAC name) (829)+TX, (E)-6-methylhept-2-en-4-ol (IUPAC name) (541)+TX, (E,Z)-tetradeca-4,10-dien-1-yl acetate (IUPAC name) (779)+TX, (Z)-dodec-7-en-1-yl acetate (IUPAC name) (285)+TX, (Z)-hexadec-11-enal (IUPAC name) (436)+TX, (Z)-hexadec-1-en-1-yl acetate (IUPAC name) (437)+TX, (Z)-hexadec-13-en-11-yn-1-yl acetate (IUPAC name) (438)+TX, (Z)-icos-13-en-10-one (IUPAC name) (448)+TX, (Z)-tetradec-7-en-1-al (IUPAC name) (782)+TX, (Z)-tetradec-9-en-1-ol (IUPAC name) (783)+TX, (Z)-tetradec-9-en-1-yl acetate (IUPAC name) (784)+TX, (7E,9Z)-dodeca-7,9-dien-1-yl acetate (IUPAC name) (283)+TX, (9Z,11E)-tetradeca-9,11-dien-1-yl acetate (IUPAC name) (780)+TX, (9Z,12E)-tetradeca-9,12-dien-1-yl acetate (IUPAC name) (781)+TX, 14-methyloctadec-1-ene (IUPAC name) (545)+TX, 4-methylnonan-5-ol with 4-methylnonan-5-one (IUPAC name) (544)+TX, alpha-multistriatin [CCN]+TX, brevicomin [CCN]+TX, codlelure [CCN]+TX, codlemone (167)+TX, cuelure (179)+TX, disparlure (277)+TX, dodec-8-en-1-yl acetate (IUPAC name) (286)+TX, dodec-9-en-1-yl acetate (IUPAC name) (287)+TX, dodeca-8+TX, 10-dien-1-yl acetate (IUPAC name) (284)+TX, dominicalure [CCN]+TX, ethyl 4-methyloctanoate (IUPAC name) (317)+TX, eugenol [CCN]+TX, frontalin [CCN]+TX, gossyplure (420)+TX, grandlure (421)+TX, grandlure I (421)+TX, grandlure II (421)+TX, grandlure III (421)+TX, grandlure IV (421)+TX, hexalure [CCN]+TX, ipsdienol [CCN]+TX, ipsenol [CCN]+TX, japonilure (481)+TX, lineatin [CCN]+TX, litlure [CCN]+TX, looplure [CCN]+TX, medlure [CCN]+TX, megatomoic acid [CCN]+TX, methyl eugenol (540)+TX, muscalure (563)+TX, octadeca-2,13-dien-1-yl acetate (IUPAC name) (588)+TX, octadeca-3,13-dien-1-yl acetate (IUPAC name) (589)+TX, orfralure [CCN]+TX, oryctalure (317)+TX, ostramone [CCN]+TX, siglure [CCN]+TX, sordidin (736)+TX, sulcatol [CCN]+TX, tetradec-11-en-1-yl acetate (IUPAC name) (785)+TX, trimedlure (839)+TX, trimedlure A (839)+TX, trimedlure B₁ (839)+TX, trimedlure B₂ (839)+TX, trimedlure C (839) and trunc-call [CCN]+TX,

an insect repellent selected from the group of substances consisting of 2-(octylthio)ethanol (IUPAC name) (591)+TX, butopyronoxyl (933)+TX, butoxy(polypropylene glycol) (936)+TX, dibutyl adipate (IUPAC name) (1046)+TX, dibutyl phthalate (1047)+TX, dibutyl succinate (IUPAC name) (1048)+TX, diethytoluamide [CCN]+TX, dimethyl carbate [CCN]+TX, dimethyl phthalate [CCN]+TX, ethyl hexanediol (1137)+TX, hexamide [CCN]+TX, methoquin-butyl (1276)+TX, methylneodecanamide [CCN]+TX, oxamate [CCN] and picaridin [CCN]+TX,

an insecticide selected from the group of substances consisting of 1-dichloro-1-nitroethane (IUPAC/Chemical Abstracts name) (1058)+TX, 1,1-dichloro-2,2-bis(4-ethylphenyl)ethane (IUPAC name) (1056), +TX, 12-dichloropropane (IUPAC/Chemical Abstracts name) (1062)+TX, 1,2-dichloropropane with 1,3-dichloropropene (IUPAC name) (1063)+TX, 1-bromo-2-chloroethane (IUPAC/Chemical Abstracts name) (916)+TX, 2,2,2-trichloro-1-(3,4-dichlorophenyl)ethyl acetate (IUPAC name) (1451)+TX, 2,2-dichlorovinyl 2-ethylsulfinylethyl methyl phosphate (IUPAC name) (1066)+TX, 2-(1,3-dithiolan-2-yl)phenyl dimethylcarbamate (IUPAC/Chemical Abstracts name) (1109)+TX, 2-(2-butoxyethoxy)ethyl thiocyanate (IUPAC/Chemical Abstracts name) (935)+TX, 2-(4,5-dimethyl-1,3-dioxolan-2-yl)phenyl methylcarbamate (IUPAC/Chemical Abstracts name) (1084)+TX, 2-(4-chloro-3,5-xylyloxy)ethanol (IUPAC name) (986)+TX, 2-chlorovinyl diethyl phosphate (IUPAC name) (984)+TX, 2-imidazolidone (IUPAC name) (1225)+TX, 2-isovalerylindan-1,3-dione (IUPAC name) (1246)+TX, 2-methyl(prop-2-ynyl)aminophenyl methylcarbamate (IUPAC name) (1284)+TX, 2-thiocyanatoethyl laurate (IUPAC name) (1433)+TX, 3-bromo-1-chloroprop-1-ene (IUPAC name) (917)+TX, 3-methyl-1-phenylpyrazol-5-yl dimethylcarbamate (IUPAC name) (1283)+TX, 4-methyl(prop-2-ynyl)amino-3,5-xylyl methylcarbamate (IUPAC name) (1285)+TX, 5,5-dimethyl-3-oxocyclohex-1-enyl dimethylcarbamate (IUPAC name) (1085)+TX, abamectin (1)+TX, acephate (2)+TX, acetamiprid (4)+TX, acethion [CCN]+TX, acetoprole [CCN]+TX, acrinathrin (9)+TX, acrylonitrile (IUPAC name) (861)+TX, alanycarb (15)+TX, aldicarb (16)+TX, aldoxycarb (863)+TX, aldrin (864)+TX, allethrin (17)+TX, allosamidin [CCN]+TX, allyxycarb (866)+TX, alpha-cypermethrin (202)+TX, alpha-ecdysone [CCN]+TX, aluminium phosphide (640)+TX, amidithion (870)+TX, amidothioate (872)+TX, aminocarb (873)+TX, amiton (875)+TX, amiton hydrogen oxalate (875)+TX, amitraz (24)+TX, anabasine (877)+TX, athidathion (883)+TX, AVI 382 (compound code)+TX, AZ 60541 (compound code)+TX, azadirachtin (41)+TX, azamethiphos (42)+TX, azinphos-ethyl (44)+TX, azinphos-methyl (45)+TX, azothoate (889)+TX, Bacillus thuringiensis delta endotoxins (52)+TX, barium hexafluorosilicate [CCN]+TX, barium polysulfide (IUPAC/Chemical Abstracts name) (892)+TX, barthrin [CCN]+TX, Bayer 22/190 (development code) (893)+TX, Bayer 22408 (development code) (894)+TX, bendiocarb (58)+TX, benfuracarb (60)+TX, bensultap (66)+TX, beta-cyfluthrn (194)+TX, beta-cypermethrin (203)+TX, bifenthrin (76)+TX, bioallethrin (78)+TX, bioallethrin S-cyclopentenyl isomer (79)+TX, bioethanomethrin [CCN]+TX, biopermethrin (908)+TX, bioresmethrin (80)+TX, bis(2-chloroethyl) ether (IUPAC name) (909)+TX, bistrifluron (83)+TX, borax (86)+TX, brofenvalerate+TX, bromfenvinfos (914)+TX, bromocyclen (918)+TX, bromo-DDT [CCN]+TX, bromophos (920)+TX, bromophos-ethyl (921)+TX, bufencarb (924)+TX, buprofezin (99)+TX, butacarb (926)+TX, butathiofos (927)+TX, butocarboxim (103)+TX, butonate (932)+TX, butoxycarboxim (104)+TX, butylpyridaben+TX, cadusafos (109)+TX, calcium arsenate [CCN]+TX, calcium cyanide (444)+TX, calcium polysulfide (IUPAC name) (111)+TX, camphechlor (941)+TX, carbanolate (943)+TX, carbaryl (115)+TX, carbofuran (118)+TX, carbon disulfide (IUPAC/Chemical Abstracts name) (945)+TX, carbon tetrachloride (IUPAC name) (946)+TX, carbophenothion (947)+TX, carbosulfan (119)+TX, cartap (123)+TX, cartap hydrochloride (123)+TX, cevadine (725)+TX, chlorbicyclen (960)+TX, chlordane (128)+TX, chlordecone (963)+TX, chlordimeform (964)+TX, chlordimeform hydrochloride (964)+TX, chlorethoxyfos (129)+TX, chlorfenapyr (130)+TX, chlorfenvinphos (131)+TX, chlorfluazuron (132)+TX, chlormephos (136)+TX, chloroform [CCN]+TX, chloropicrin (141)+TX, chlorphoxim (989)+TX, chlorprazophos (990)+TX, chlorpyrifos (145)+TX, chlorpyrifos-methyl (146)+TX, chlorthiophos (994)+TX, chromafenozide (150)+TX, cinerin I (696)+TX, cinerin II (696)+TX, cinerins (696)+TX, cis-resmethrin+TX, cismethrin (80)+TX, clocythrin+TX, cloethocarb (999)+TX, closantel [CCN]+TX, clothianidin (165)+TX, copper acetoarsenite [CCN]+TX, copper arsenate [CCN]+TX, copper oleate [CCN]+TX, coumaphos (174)+TX, coumithoate (1006)+TX, crotamiton [CCN]+TX, crotoxyphos (1010)+TX, crufomate (1011)+TX, cryolite (177)+TX, CS 708 (development code) (1012)+TX, cyanofenphos (1019)+TX, cyanophos (184)+TX, cyanthoate (1020)+TX, cyclethrin [CCN]+TX, cycloprothrin (188)+TX, cyfluthrin (193)+TX, cyhalothrin (196)+TX, cypermethrin (201)+TX, cyphenothrin (206)+TX, cyromazine (209)+TX, cythioate [CCN]+TX, d-limonene [CCN]+TX, d-tetramethrin (788)+TX, DAEP (1031)+TX, dazomet (216)+TX, DDT (219)+TX, decarbofuran (1034)+TX, deltamethrin (223)+TX, demephion (1037)+TX, demephion-O (1037)+TX, demephion-S (1037)+TX, demeton (1038)+TX, demeton-methyl (224)+TX, demeton-O (1038)+TX, demeton-O-methyl (224)+TX, demeton-S (1038)+TX, demeton-S-methyl (224)+TX, demeton-S-methylsulphon (1039)+TX, diafenthiuron (226)+TX, dialifos (1042)+TX, diamidafos (1044)+TX, diazinon (227)+TX, dicapthon (1050)+TX, dichlofenthion (1051)+TX, dichlorvos (236)+TX, dicliphos+TX, dicresyl [CCN]+TX, dicrotophos (243)+TX, dicyclanil (244)+TX, dieldrin (1070)+TX, diethyl 5-methylpyrazol-3-yl phosphate (IUPAC name) (1076)+TX, diflubenzuron (250)+TX, dilor [CCN]+TX, dimefluthrin [CCN]+TX, dimefox (1081)+TX, dimetan (1085)+TX, dimethoate (262)+TX, dimethrin (1083)+TX, dimethylvinphos (265)+TX, dimetilan (1086)+TX, dinex (1089)+TX, dinex-diclexine (1089)+TX, dinoprop (1093)+TX, dinosam (1094)+TX, dinoseb (1095)+TX, dinotefuran (271)+TX, diofenolan (1099)+TX, dioxabenzofos (1100)+TX, dioxacarb (1101)+TX, dioxathion (1102)+TX, disulfoton (278)+TX, dithicrofos (1108)+TX, DNOC (282)+TX, doramectin [CCN]+TX, DSP (1115)+TX, ecdysterone [CCN]+TX, El 1642 (development code) (1118)+TX, emamectin (291)+TX, emamectin benzoate (291)+TX, EMPC (1120)+TX, empenthrin (292)+TX, endosulfan (294)+TX, endothlon (1121)+TX, endrin (1122)+TX, EPBP (1123)+TX, EPN (297)+TX, epofenonane (1124)+TX, eprinomectin [CCN]+TX, esfenvalerate (302)+TX, etaphos [CCN]+TX, ethiofencarb (308)+TX, ethion (309)+TX, ethiprole (310)+TX, ethoate-methyl (1134)+TX, ethoprophos (312)+TX, ethyl formate (IUPAC name) [CCN]+TX, ethyl-DDD (1056)+TX, ethylene dibromide (316)+TX, ethylene dichloride (chemical name) (1136)+TX, ethylene oxide [CCN]+TX, etofenprox (319)+TX, etrimfos (1142)+TX, EXD (1143)+TX, famphur (323)+TX, fenamiphos (326)+TX, fenazaflor (1147)+TX, fenchlorphos (1148)+TX, fenethacarb (1149)+TX, fenfluthrin (1150)+TX, fenitrothion (335)+TX, fenobucarb (336)+TX, fenoxacrim (1153)+TX, fenoxycarb (340)+TX, fenpirithrin (1155)+TX, fenpropathrin (342)+TX, fenpyrad+TX, fensulfothion (1158)+TX, fenthion (346)+TX, fenthion-ethyl [CCN]+TX, fenvalerate (349)+TX, fipronil (354)+TX, flonicamid (358)+TX, flubendiamide (CAS. Reg. No.: 272451-65-7)+TX, flucofuron (1168)+TX, flucycloxuron (366)+TX, flucythrinate (367)+TX, fluenetil (1169)+TX, flufenerim [CCN]+TX, flufenoxuron (370)+TX, flufenprox (1171)+TX, flumethrin (372)+TX, fluvalinate (1184)+TX, FMC 1137 (development code) (1185)+TX, fonofos (1191)+TX, formetanate (405)+TX, formetanate hydrochloride (405)+TX, formothion (1192)+TX, formparanate (1193)+TX, fosmethilan (1194)+TX, fospirate (1195)+TX, fosthiazate (408)+TX, fosthietan (1196)+TX, furathiocarb (412)+TX, furethrin (1200)+TX, gamma-cyhalothrin (197)+TX, gamma-HCH (430)+TX, guazatine (422)+TX, guazatine acetates (422)+TX, GY-81 (development code) (423)+TX, halfenprox (424)+TX, halofenozide (425)+TX, HCH (430)+TX, HEOD (1070)+TX, heptachlor (1211)+TX, heptenophos (432)+TX, heterophos [CCN]+TX, hexaflumuron (439)+TX, HHDN (864)+TX, hydramethylnon (443)+TX, hydrogen cyanide (444)+TX, hydroprene (445)+TX, hyquincarb (1223)+TX, imidacloprid (458)+TX, imiprothrin (460)+TX, indoxacarb (465)+TX, iodomethane (IUPAC name) (542)+TX, IPSP (1229)+TX, isazofos (1231)+TX, isobenzan (1232)+TX, isocarbophos (473)+TX, isodrin (1235)+TX, isofenphos (1236)+TX, isolane (1237)+TX, isoprocarb (472)+TX, isopropyl O-(methoxyaminothiophosphoryl)salicylate (IUPAC name) (473)+TX, isoprothiolane (474)+TX, isothioate (1244)+TX, isoxathion (480)+TX, ivermectin [CCN]+TX, jasmolin I (696)+TX, jasmolin II (696)+TX, jodfenphos (1248)+TX, juvenile hormone I [CCN]+TX, juvenile hormone II [CCN]+TX, juvenile hormone III [CCN]+TX, kelevan (1249)+TX, kinoprene (484)+TX, lambda-cyhalothrin (198)+TX, lead arsenate [CCN]+TX, lepimectin (CCN)+TX, leptophos (1250)+TX, lindane (430)+TX, lirimfos (1251)+TX, lufenuron (490)+TX, lythidathion (1253)+TX, m-cumenyl methylcarbamate (IUPAC name) (1014)+TX, magnesium phosphide (IUPAC name) (640)+TX, malathion (492)+TX, malonoben (1254)+TX, mazidox (1255)+TX, mecarbam (502)+TX, mecarphon (1258)+TX, menazon (1260)+TX, mephosfolan (1261)+TX, mercurous chloride (513)+TX, mesulfenfos (1263)+TX, metaflumizone (CCN)+TX, metam (519)+TX, metam-potassium (519)+TX, metam-sodium (519)+TX, methacrifos (1266)+TX, methamidophos (527)+TX, methanesulfonyl fluoride (IUPAC/Chemical Abstracts name) (1268)+TX, methidathion (529)+TX, methiocarb (530)+TX, methocrotophos (1273)+TX, methomyl (531)+TX, methoprene (532)+TX, methoquin-butyl (1276)+TX, methothrin (533)+TX, methoxychlor (534)+TX, methoxyfenozide (535)+TX, methyl bromide (537)+TX, methyl isothiocyanate (543)+TX, methylchloroform [CCN]+TX, methylene chloride [CCN]+TX, metofluthrin [CCN]+TX, metolcarb (550)+TX, metoxadiazone (1288)+TX, mevinphos (556)+TX, mexacarbate (1290)+TX, milbemectin (557)+TX, milbemycin oxime [CCN]+TX, mipafox (1293)+TX, mirex (1294)+TX, monocrotophos (561)+TX, morphothion (1300)+TX, moxidectin [CCN]+TX, naftalofos [CCN]+TX, naled (567)+TX, naphthalene (IUPAC/Chemical Abstracts name) (1303)+TX, NC-170 (development code) (1306)+TX, NC-184 (compound code)+TX, nicotine (578)+TX, nicotine sulfate (578)+TX, nifluridide (1309)+TX, nitenpyram (579)+TX, nithiazine (1311)+TX, nitrilacarb (1313)+TX, nitrilacarb 1:1 zinc chloride complex (1313)+TX, NNI-0101 (compound code)+TX, NNI-0250 (compound code)+TX, nornicotine (traditional name) (1319)+TX, novaluron (585)+TX, noviflumuron (586)+TX, 0-5-dichloro-4-iodophenyl O-ethyl ethylphosphonothioate (IUPAC name) (1057)+TX, O,O-diethyl O-4-methyl-2-oxo-2H-chromen-7-yl phosphorothioate (IUPAC name) (1074)+TX, O,O-diethyl O-6-methyl-2-propylpyrimidin-4-yl phosphorothioate (IUPAC name) (1075)+TX, O,O,O′,O′-tetrapropyl dithiopyrophosphate (IUPAC name) (1424)+TX, oleic acid (IUPAC name) (593)+TX, omethoate (594)+TX, oxamyl (602)+TX, oxydemeton-methyl (609)+TX, oxydeprofos (1324)+TX, oxydisulfoton (1325)+TX, pp′-DDT (219)+TX, para-dichlorobenzene [CCN]+TX, parathion (615)+TX, parathion-methyl (616)+TX, penfluron [CCN]+TX, pentachlorophenol (623)+TX, pentachlorophenyl laurate (IUPAC name) (623)+TX, permethrin (626)+TX, petroleum oils (628)+TX, PH 60-38 (development code) (1328)+TX, phenkapton (1330)+TX, phenothrin (630)+TX, phenthoate (631)+TX, phorate (636)+TX, phosalone (637)+TX, phosfolan (1338)+TX, phosmet (638)+TX, phosnichlor (1339)+TX, phosphamidon (639)+TX, phosphine (IUPAC name) (640)+TX, phoxim (642)+TX, phoxim-methyl (1340)+TX, pirimetaphos (1344)+TX, pirimicarb (651)+TX, pirimiphos-ethyl (1345)+TX, pirimiphos-methyl (652)+TX, polychlorodicyclopentadiene isomers (IUPAC name) (1346)+TX, polychloroterpenes (traditional name) (1347)+TX, potassium arsenite [CCN]+TX, potassium thiocyanate [CCN]+TX, prallethrin (655)+TX, precocene I [CCN]+TX, precocene II [CCN]+TX, precocene III [CCN]+TX, primidophos (1349)+TX, profenofos (662)+TX, profluthrin [CCN]+TX, promacyl (1354)+TX, promecarb (1355)+TX, propaphos (1356)+TX, propetamphos (673)+TX, propoxur (678)+TX, prothidathion (1360)+TX, prothiofos (686)+TX, prothoate (1362)+TX, protrifenbute [CCN]+TX, pymetrozine (688)+TX, pyraclofos (689)+TX, pyrazophos (693)+TX, pyresmethrin (1367)+TX, pyrethrin I (696)+TX, pyrethrin II (696)+TX, pyrethrins (696)+TX, pyridaben (699)+TX, pyridalyl (700)+TX, pyridaphenthion (701)+TX, pyrimidifen (706)+TX, pyrimitate (1370)+TX, pyriproxyfen (708)+TX, quassia [CCN]+TX, quinalphos (711)+TX, quinalphos-methyl (1376)+TX, quinothion (1380)+TX, quintiofos (1381)+TX, R-1492 (development code) (1382)+TX, rafoxanide [CCN]+TX, resmethrin (719)+TX, rotenone (722)+TX, RU 15525 (development code) (723)+TX, RU 25475 (development code) (1386)+TX, ryania (1387)+TX, ryanodine (traditional name) (1387)+TX, sabadilla (725)+TX, schradan (1389)+TX, sebufos+TX, selamectin [CCN]+TX, SI-0009 (compound code)+TX, SI-0205 (compound code)+TX, SI-0404 (compound code)+TX, SI-0405 (compound code)+TX, silafluofen (728)+TX, SN 72129 (development code) (1397)+TX, sodium arsenite [CCN]+TX, sodium cyanide (444)+TX, sodium fluoride (IUPAC/Chemical Abstracts name) (1399)+TX, sodium hexafluorosilicate (1400)+TX, sodium pentachlorophenoxide (623)+TX, sodium selenate (IUPAC name) (1401)+TX, sodium thiocyanate [CCN]+TX, sophamide (1402)+TX, spinosad (737)+TX, spiromesifen (739)+TX, spirotetrmat (CCN)+TX, sulcofuron (746)+TX, sulcofuron-sodium (746)+TX, sulfluramid (750)+TX, sulfotep (753)+TX, sulfuryl fluoride (756)+TX, sulprofos (1408)+TX, tar oils (758)+TX, tau-fluvalinate (398)+TX, tazimcarb (1412)+TX, TDE (1414)+TX, tebufenozide (762)+TX, tebufenpyrad (763)+TX, tebupirimfos (764)+TX, teflubenzuron (768)+TX, tefluthrin (769)+TX, temephos (770)+TX, TEPP (1417)+TX, terallethrin (1418)+TX, terbam+TX, terbufos (773)+TX, tetrachloroethane [CCN]+TX, tetrachlorvinphos (777)+TX, tetramethrin (787)+TX, theta-cypermethrin (204)+TX, thiacloprid (791)+TX, thiafenox+TX, thiamethoxam (792)+TX, thicrofos (1428)+TX, thiocarboxime (1431)+TX, thiocyclam (798)+TX, thiocyclam hydrogen oxalate (798)+TX, thiodicarb (799)+TX, thiofanox (800)+TX, thiometon (801)+TX, thionazin (1434)+TX, thiosultap (803)+TX, thiosultap-sodium (803)+TX, thuringiensin [CCN]+TX, tolfenpyrad (809)+TX, tralomethrin (812)+TX, transfluthrin (813)+TX, transpermethrin (1440)+TX, triamiphos (1441)+TX, triazamate (818)+TX, triazophos (820)+TX, triazuron+TX, trichlorfon (824)+TX, trichlormetaphos-3 [CCN]+TX, trichloronat (1452)+TX, trifenofos (1455)+TX, triflumuron (835)+TX, trimethacarb (840)+TX, triprene (1459)+TX, vamidothion (847)+TX, vaniliprole [CCN]+TX, veratridine (725)+TX, veratrine (725)+TX, XMC (853)+TX, xylylcarb (854)+TX, YI-5302 (compound code)+TX, zeta-cypermethrin (205)+TX, zetamethrin+TX, zinc phosphide (640)+TX, zolaprofos (1469) and ZXI 8901 (development code) (858)+TX, cyantraniliprole [736994-63-19+TX, chlorantraniliprole [500008-45-7]+TX, cyenopyrafen [560121-52-0]+TX, cyflumetofen [400882-07-7]+TX, pyrifluquinazon [337458-27-2]+TX, spinetoram [187166-40-1+187166-15-0]+TX, spirotetramat [203313-25-1]+TX, sulfoxaflor [946578-00-3]+TX, flufiprole [704886-18-0]+TX, meperfluthrin [915288-13-0]+TX, tetramethylfluthrin [84937-88-2]+TX, triflumezopyrim (disclosed in WO 2012/092115)+TX, fluxametamide (WO 2007/026965)+TX, epsilon-metofluthrin [240494-71-7]+TX, epsilon-momfluorothrin [1065124-65-3]+TX, fluazaindolizine [1254304-22-7]+TX, chloroprallethrin [399572-87-3]+TX, fluxametamide [928783-29-3]+TX, cyhalodiamide [1262605-53-7]+TX, tioxazafen [330459-31-9]+TX, broflanilide [1207727-04-5]+TX, flufiprole [704886-18-0]+TX, cyclaniliprole [1031756-98-5]+TX, tetraniliprole [1229654-66-3]+TX, guadipyr (described in WO2010/060231)+TX, cycloxaprid (described in WO2005/077934)+TX,

a molluscicide selected from the group of substances consisting of bis(tributyltin) oxide (IUPAC name) (913)+TX, bromoacetamide [CCN]+TX, calcium arsenate [CCN]+TX, cloethocarb (999)+TX, copper acetoarsenite [CCN]+TX, copper sulfate (172)+TX, fentin (347)+TX, ferric phosphate (IUPAC name) (352)+TX, metaldehyde (518)+TX, methiocarb (530)+TX, niclosamide (576)+TX, niclosamide-olamine (576)+TX, pentachlorophenol (623)+TX, sodium pentachlorophenoxide (623)+TX, tazimcarb (1412)+TX, thiodicarb (799)+TX, tributyltin oxide (913)+TX, trifenmorph (1454)+TX, trimethacarb (840)+TX, triphenyltin acetate (IUPAC name) (347) and triphenyltin hydroxide (IUPAC name) (347)+TX, pyriprole [394730-71-3]+TX, a nematicide selected from the group of substances consisting of AKD-3088 (compound code)+TX, 1,2-dibromo-3-chloropropane (IUPAC/Chemical Abstracts name) (1045)+TX, 1,2-dichloropropane (IUPAC/Chemical Abstracts name) (1062)+TX, 1,2-dichloropropane with 1,3-dichloropropene (IUPAC name) (1063)+TX, 1,3-dichloropropene (233)+TX, 3,4-dichlorotetrahydrothiophene 1,1-dioxide (IUPAC/Chemical Abstracts name) (1065)+TX, 3-(4-chlorophenyl)-5-methylrhodanine (IUPAC name) (980)+TX, 5-methyl-6-thioxo-1,3,5-thiadiazinan-3-ylacetic acid (IUPAC name) (1286)+TX, 6-sopentenylaminopurine (210)+TX, abamectin (1)+TX, acetoprole [CCN]+TX, alanycarb (15)+TX, aldicarb (16)+TX, aldoxycarb (863)+TX, AZ 60541 (compound code)+TX, benclothiaz [CCN]+TX, benomyl (62)+TX, butylpyridaben+TX, cadusafos (109)+TX, carbofuran (118)+TX, carbon disulfide (945)+TX, carbosulfan (119)+TX, chloropicrin (141)+TX, chlorpyrifos (145)+TX, cloethocarb (999)+TX, cytokinins (210)+TX, dazomet (216)+TX, DBCP (1045)+TX, DCIP (218)+TX, diamidafos (1044)+TX, dichlofenthion (1051)+TX, dicliphos+TX, dimethoate (262)+TX, doramectin [CCN]+TX, emamectin (291)+TX, emamectin benzoate (291)+TX, eprinomectin [CCN]+TX, ethoprophos (312)+TX, ethylene dibromide (316)+TX, fenamiphos (326)+TX, fenpyrad+TX, fensulfothion (1158)+TX, fosthiazate (408)+TX, fosthietan (1196)+TX, furfural [CCN]+TX, GY-81 (development code) (423)+TX, heterophos [CCN]+TX, iodomethane (IUPAC name) (542)+TX, isamidofos (1230)+TX, isazofos (1231)+TX, ivermectin [CCN]+TX, kinetin (210)+TX, mecarphon (1258)+TX, metam (519)+TX, metam-potassium (519)+TX, metam-sodium (519)+TX, methyl bromide (537)+TX, methyl isothiocyanate (543)+TX, milbemycin oxime [CCN]+TX, moxidectin [CCN]+TX, Myrothecium verrucaria composition (565)+TX, NC-184 (compound code)+TX, oxamyl (602)+TX, phorate (636)+TX, phosphamidon (639)+TX, phosphocarb [CCN]+TX, sebufos+TX, selamectin [CCN]+TX, spinosad (737)+TX, terbam+TX, terbufos (773)+TX, tetrachlorothiophene (IUPAC/Chemical Abstracts name) (1422)+TX, thiafenox+TX, thionazin (1434)+TX, triazophos (820)+TX, triazuron+TX, xylenols [CCN]+TX, YI-5302 (compound code) and zeatin (210)+TX, fluensulfone [318290-98-1]+TX,

a nitrification inhibitor selected from the group of substances consisting of potassium ethylxanthate [CCN] and nitrapyrin (580)+TX,

a plant activator selected from the group of substances consisting of acibenzolar (6)+TX, acibenzolar-S-methyl (6)+TX, probenazole (658) and Reynoutia sachalinensis extract (720)+TX, a rodenticide selected from the group of substances consisting of 2-isovalerylindan-1,3-dione (IUPAC name) (1246)+TX, 4-(quinoxalin-2-ylamino)benzenesulfonamide (IUPAC name) (748)+TX, alpha-chlorohydrin [CCN]+TX, aluminium phosphide (640)+TX, antu (880)+TX, arsenous oxide (882)+TX, barium carbonate (891)+TX, bisthiosemi (912)+TX, brodifacoum (89)+TX, bromadiolone (91)+TX, bromethalin (92)+TX, calcium cyanide (444)+TX, chloralose (127)+TX, chlorophacinone (140)+TX, cholecalciferol (850)+TX, coumachlor (1004)+TX, coumafuryl (1005)+TX, coumatetralyl (175)+TX, crimidine (1009)+TX, difenacoum (246)+TX, difethialone (249)+TX, diphacinone (273)+TX, ergocalciferol (301)+TX, flocoumafen (357)+TX, fluoroacetamide (379)+TX, flupropadine (1183)+TX, flupropadine hydrochloride (1183)+TX, gamma-HCH (430)+TX, HCH (430)+TX, hydrogen cyanide (444)+TX, iodomethane (IUPAC name) (542)+TX, lindane (430)+TX, magnesium phosphide (IUPAC name) (640)+TX, methyl bromide (537)+TX, norbormide (1318)+TX, phosacetim (1336)+TX, phosphine (IUPAC name) (640)+TX, phosphorus [CCN]+TX, pindone (1341)+TX, potassium arsenite [CCN]+TX, pyrinuron (1371)+TX, scillroside (1390)+TX, sodium arsenite [CCN]+TX, sodium cyanide (444)+TX, sodium fluoroacetate (735)+TX, strychnine (745)+TX, thallium sulfate [CCN]+TX, warfarin (851) and zinc phosphide (640)+TX,

a synergist selected from the group of substances consisting of 2-(2-butoxyethoxy)ethyl piperonylate (IUPAC name) (934)+TX, 5-(1,3-benzodioxol-5-yl)-3-hexylcyclohex-2-enone (IUPAC name) (903)+TX, farnesol with nerolidol (324)+TX, MB-599 (development code) (498)+TX, MGK 264 (development code) (296)+TX, piperonyl butoxide (649)+TX, piprotal (1343)+TX, propyl isomer (1358)+TX, S421 (development code) (724)+TX, sesamex (1393)+TX, sesasmolin (1394) and sulfoxide (1406)+TX,

an animal repellent selected from the group of substances consisting of anthraquinone (32)+TX, chloralose (127)+TX, copper naphthenate [CCN]+TX, copper oxychloride (171)+TX, diazinon (227)+TX, dicyclopentadiene (chemical name) (1069)+TX, guazatine (422)+TX, guazatine acetates (422)+TX, methiocarb (530)+TX, pyridin-4-amine (IUPAC name) (23)+TX, thiram (804)+TX, trimethacarb (840)+TX, zinc naphthenate [CCN] and ziram (856)+TX,

a virucide selected from the group of substances consisting of imanin [CCN] and ribavirin [CCN]+TX,

a wound protectant selected from the group of substances consisting of mercuric oxide (512)+TX, octhilinone (590) and thiophanate-methyl (802)+TX,

and biologically active compounds selected from the group consisting of azaconazole (60207-31-0]+TX, benzovindiflupyr [1072957-71-1]+TX, bitertanol [70585-36-3]+TX, bromuconazole [116255-48-2]+TX, cyproconazole [94361-06-5]+TX, difenoconazole [119446-68-3]+TX, diniconazole [83657-24-3]+TX, epoxiconazole [106325-08-0]+TX, fenbuconazole [114369-43-6]+TX, fluquinconazole [136426-54-5]+TX, flusilazole [85509-19-9]+TX, flutriafol [76674-21-0]+TX, hexaconazole [79983-71-4]+TX, imazalil [35554-44-0]+TX, imibenconazole [86598-92-7]+TX, ipconazole [125225-28-7]+TX, metconazole [125116-23-6]+TX, myclobutanil [88671-89-0]+TX, pefurazoate [101903-30-4]+TX, penconazole [66246-88-6]+TX, prothioconazole [178928-70-6]+TX, pyrifenox [88283-41-4]+TX, prochloraz [67747-09-5]+TX, propiconazole [60207-90-1]+TX, simeconazole [149508-90-7]+TX, tebuconazole [107534-96-3]+TX, tetraconazole [112281-77-3]+TX, tradimefon [43121-43-3]+TX, triadimenol [55219-65-3]+TX, triflumizole [99387-89-0]+TX, triticonazole [131983-72-7]+TX, ancymidol [12771-68-5]+TX, fenarimol [60168-88-9]+TX, nuarimol [63284-71-9]+TX, bupirimate [41483-43-6]+TX, dimethirimol [5221-53-4]+TX, ethirimol [23947-60-6]+TX, dodemorph [1593-77-7]+TX, fenpropidine [67306-00-7]+TX, fenpropimorph [67564-91-4]+TX, spiroxamine [118134-30-8]+TX, tridemorph [81412-43-3]+TX, cyprodinil [121552-61-2]+TX, mepanipyrim [110235-47-7]+TX, pyrimethanil [53112-28-0]+TX, fenpiclonil [74738-17-3]+TX, fludioxonil [131341-86-1]+TX, benalaxyl [71626-11-4]+TX, furalaxyl [57646-30-7]+TX, metalaxyl [57837-19-1]+TX, R-metalaxyl [70630-17-0]+TX, ofurace [58810-48-3]+TX, oxadixyl [77732-09-3]+TX, benomyl [17804-35-2]+TX, carbendazim [10605-21-7]+TX, debacarb [62732-91-6]+TX, fuberidazole [3878-19-1]+TX, thiabendazole [148-79-8]+TX, chlozolinate [84332-86-5]+TX, dichlozoline [24201-58-9]+TX, iprodione [36734-19-7]+TX, myclozoline [54864-61-8]+TX, procymidone [32809-16-8]+TX, vinclozoline [50471-44-8]+TX, boscalid [188425-85-6]+TX, carboxin [5234-68-4]+TX, fenfuram [24691-80-3]+TX, fenpicoxamid [517875-34-2]+TX, flutolanil [66332-96-5]+TX, mepronil [55814-41-0]+TX, oxycarboxin [5259-88-1]+TX, penthiopyrad [183675-82-3]+TX, thifluzamide [130000-40-7]+TX, guazatine [108173-90-6]+TX, dodine [2439-10-3] [112-65-2] (free base)+TX, iminoctadine [13516-27-3]+TX, azoxystrobin [131860-33-8]+TX, dimoxystrobin [149961-52-4]+TX, enestroburin {Proc. BCPC, Int. Congr., Glasgow, 2003, 1, 93}+TX, fluoxastrobin [361377-29-9]+TX, kresoxim-methyl [143390-89-0]+TX, metominostrobin [133408-50-1]+TX, trifloxystrobin [141517-21-7]+TX, orysastrobin [248593-16-0]+TX, picoxystrobin [117428-22-5]+TX, pyraclostrobin [175013-18-0]+TX, ferbam [14484-64-1]+TX, mancozeb [8018-01-7]+TX, maneb [12427-38-2]+TX, metiram [9006-42-2]+TX, propineb [12071-83-9]+TX, thiram [137-26-8]+TX, zineb [12122-67-7]+TX, ziram [137-30-4]+TX, captafol [2425-06-1]+TX, captan [133-06-2]+TX, dichlofluanid [1085-98-9]+TX, fluoroimide [41205-21-4]+TX, folpet [133-07-3]+TX, tolylfluanid [731-27-1]+TX, bordeaux mixture [8011-63-0]+TX, copperhydroxid [20427-59-2]+TX, copperoxychlorid [1332-40-7]+TX, coppersulfat [7758-98-7]+TX, copperoxid [1317-39-1]+TX, mancopper [53988-93-5]+TX, oxine-copper [10380-28-6]+TX, dinocap [131-72-6]+TX, nitrothal-isopropyl [10552-74-6]+TX, edifenphos [17109-49-8]+TX, iprobenphos [26087-47-8]+TX, isoprothiolane [50512-35-1]+TX, phosdiphen [36519-00-3]+TX, pyrazophos [13457-18-6]+TX, tolclofos-methyl [57018-04-9]+TX, acibenzolar-S-methyl [135158-54-2]+TX, anilazine [101-05-3]+TX, benthiavalicarb [413615-35-7]+TX, blasticidin-S [2079-00-7]+TX, chinomethionat [2439-01-2]+TX, chloroneb [2675-77-6]+TX, chlorothalonil [1897-45-6]+TX, cyflufenamid [180409-60-3]+TX, cymoxanil [57966-95-7]+TX, dichlone [117-80-6]+TX, diclocymet [139920-32-4]+TX, diclomezine [62865-36-5]+TX, dicloran [99-30-9]+TX, diethofencarb [87130-20-9]+TX, dimethomorph [110488-70-5]+TX, SYP-LI90 (Flumorph) [211867-47-9]+TX, dithianon [3347-22-6]+TX, ethaboxam [162650-77-3]+TX, etridiazole [2593-15-9]+TX, famoxadone [131807-57-3]+TX, fenamidone [161326-34-7]+TX, fenoxanil [115852-48-7]+TX, fentin [668-34-8]+TX, ferimzone [89269-64-7]+TX, fluazinam [79622-59-6]+TX, flupicolide [239110-15-7]+TX, flusulfamide [106917-52-6]+TX, fenhexamid [126833-17-8]+TX, fosetyl-aluminium [39148-24-8]+TX, hymexazol [10004-44-1]+TX, iprovalicarb [140923-17-7]+TX, IKF-916 (Cyazofamid) [120116-88-3]+TX, kasugamycin [6980-18-3]+TX, methasulfocarb [66952-49-6]+TX, metrafenone [220899-03-6]+TX, oxathiapiprolin [1003318-67-9]+TX, pencycuron [66063-05-6]+TX, phthalide [27355-22-2]+TX, polyoxins [11113-80-7]+TX, probenazole [27605-76-1]+TX, propamocarb [25606-41-1]+TX, proquinazid [189278-12-4]+TX, pyroquilon [57369-32-1]+TX, quinoxyfen [124495-18-7]+TX, quintozene [82-68-8]+TX, sulfur [7704-34-9]+TX, tiadinil [223580-51-6]+TX, triazoxide [72459-58-6]+TX, tricydazole [41814-78-2]+TX, triforine [26644-46-2]+TX, validamycin [37248-47-8]+TX, zoxamide (RH7281) [156052-68-5]+TX, mandipropamid [374726-62-2]+TX, isopyrazam [881685-58-1]+TX, sedaxane [874967-67-6]+TX, 3-difluoromethyl-1-methyl-1H-pyrazole-4-carboxylic acid (9-dichloromethylene-1,2,3,4-tetrahydro-1,4-methano-naphthalen-5-yl)-amide (disclosed in WO 2007/048556)+TX, 3-difluoromethyl-1-methyl-1H-pyrazole-4-carboxylic acid (3′,4′,5′-trifluoro-biphenyl-2-yl)-amide (disclosed in WO 2006/087343)+TX, [(3S,4R,4aR,6S,6aS,12R,12aS,12bS)-3-[(cyclopropylcarbonyl)oxy]-1,3,4,4a,5,6,6a,12,12a,12b-decahydro-6,12-dihydroxy-4,6a,12b-trimethyl-11-oxo-9-(3-pyridinyl)-2H,11Hnaphtho[2,1-b]pyrano[3,4-e]pyran-4-ylmethyl-cyclopropanecarboxylate [915972-17-7]+TX and 1,3,5-trimethyl-N-(2-methyl-1-oxopropyl)-N-[3-(2-methylpropyl)-4-[2,2,2-trifluoro-1-methoxy-1-(trifluoromethyl)ethyl]phenyl]-1H-pyrazole-4-carboxamide [926914-55-8]+TX; lancotrione [1486617-21-3]+TX, florpyrauxifen [943832-81-3]]+TX, ipfentrifluconazole [1417782-08-1]+TX, mefentrifluconazole [1417782-03-6]+TX, quinofumelin [861647-84-9]]+TX, chloroprallethrin [399572-87-3]]+TX, cyhalodiamide [1262605-53-7]]+TX, fluazaindolizine [1254304-22-7]+TX, fluxametamide [928783-29-3]+TX, epsilon-metofluthrin [240494-71-7]]+TX, epsilon-momfluorothrin [1065124-65-3]+TX, pydiflumetofen [1228284-64-7]+TX, kappa-bifenthrin [439680-76-9]+TX, broflanilide [1207727-04-5]+TX, dicloromezotiaz [1263629-39-5]+TX, dipymetitrone [16114-35-5]+TX, pyraziflumid [942515-63-1] and kappa-tefluthrin [391634-71-2]+TX; and

microbials including: Acinetobacter iwoffii+TX, Acremonium alternatum+TX+TX, Acremonium cephalosporium+TX+TX, Acremonium diospyri+TX, Acremonium obciavatum+TX, Adoxophyes orana granulovirus (AdoxGV) (Capex®)+TX, Agrobacterium radiobacter strain K84 (Galitrol-A®)+TX, Alternaria alternate+TX, Alternaria cassia+TX, Alternaria destruens (Smolder®)+TX, Ampelomyces quisqualis (AQ10®)+TX, Aspergillus flavus AF36 (AF36®)+TX, Aspergillus flavus NRRL 21882 (Aflaguard®)+TX, Aspergillus spp.+TX, Aureobasidium pullulans+TX, Azospirillum+TX, (MicroAZ®+TX, TAZO B®)+TX, Azotobacter+TX, Azotobacter chroocuccum (Azotomeal®)+TX, Azotobacter cysts (Bionatural Blooming Blossoms®)+TX, Bacillus amyloliquefaciens+TX, Bacillus cereus+TX, Bacillus chitinosporus strain CM-1+TX, Bacillus chitinosporus strain AQ746+TX, Bacillus licheniformis strain HB-2 (Biostart™ Rhizoboost®)+TX, Bacillus licheniformis strain 3086 (EcoGuard®+TX, Green Releaf®)+TX, Bacillus circulans+TX, Bacillus firmus (BioSafe®+TX, BioNem-WP®+TX, VOTIVO®)+TX, Bacillus firmus strain 1-1582+TX, Bacillus macerans+TX, Bacillus marismortui+TX, Bacillus megaterium+TX, Bacillus mycoides strain AQ726+TX, Bacillus papillae (Milky Spore Powder®)+TX, Bacillus pumilus spp.+TX, Bacillus pumilus strain GB34 (Yield Shield®)+TX, Bacillus pumilus strain AQ717+TX, Bacillus pumilus strain QST 2808 (Sonata®+TX, Ballad Plus®)+TX, Bacillus spahericus (VectoLex®)+TX, Bacillus spp.+TX, Bacillus spp. strain AQ175+TX, Bacillus spp. strain AQ177+TX, Bacillus spp. strain AQ178+TX, Bacillus subtilis strain QST 713 (CEASE®+TX, Serenade®+TX, Rhapsody®)+TX, Bacillus subtilis strain QST 714 (JAZZ®)+TX, Bacillus subtilis strain AQ153+TX, Bacillus subtilis strain AQ743+TX, Bacillus subtilis strain QST3002+TX, Bacillus subtilis strain QST3004+TX, Bacillus subtilis var. amyloliquefaciens strain FZB24 (Taegro®+TX, Rhizopro®)+TX, Bacillus thuringiensis Cry 2Ae+TX, Bacillus thuringiensis Cry1Ab+TX, Bacillus thuringiensis aizawai GC 91 (Agree®)+TX, Bacillus thuringiensis israelensis (BMP123®+TX, Aquaba®+TX, VectoBac®)+TX, Bacillus thuringiensis kurstaki (Javelin®+TX, Deliver®+TX, CryMax®+TX, Bonide®+TX, Scutela WP®+TX, Turlav WP®+TX, Astuto®+TX, Dipel WP®+TX, Biobit®+TX, Foray®)+TX, Bacillus thuringiensis kurstaki BMP 123 (Baritone®)+TX, Bacillus thuringiensis kurstaki HD-1 (Bioprotec-CAF/3P®)+TX, Bacillus thuringiensis strain BD#32+TX, Bacillus thuringiensis strain AQ52+TX, Bacillus thuringiensis var. aizawai (XenTari®+TX, DiPel®)+TX, bacteria spp. (GROWMEND®+TX, GROWSWEET®+TX, Shootup®)+TX, bacteriophage of Clavipacter michiganensis (AgriPhage®)+TX, Bakflor®+TX, Beauveria bassiana (Beaugenic®+TX, Brocaril WP®)+TX, Beauveria bassiana GHA (Mycotrol ES®+TX, Mycotrol O®+TX, BotaniGuard®)+TX, Beauveria brongniartii (Engerlingspilz®+TX, Schweizer Beauveria®+TX, Melocont®)+TX, Beauveria spp.+TX, Botrytis cineria+TX, Bradyrhizobium japonicum (TerraMax®)+TX, Brevibacillus brevis+TX, Bacillus thuringiensis tenebrionis (Novodotr)+TX, BtBooster+TX, Burkholderia cepacia (Deny®+TX, Intercept®+TX, Blue Circle®)+TX, Burkholderia gladii+TX, Burkholderia gladioli+TX, Burkholderia spp.+TX, Canadian thistle fungus (CBH Canadian Bioherbicide®)+TX, Candida butyri+TX, Candida famata+TX, Candida fructus+TX, Candida glabrata+TX, Candida guilliermondii+TX, Candida melibiosica+TX, Candida oleophila strain O+TX, Candida parapsilosis+TX, Candida pelliculosa+TX, Candida pulcherrima+TX, Candida reukaufii+TX, Candida saitoana (Bio-Coat®+TX, Biocure®)+TX, Candida sake+TX, Candida spp.+TX, Candida tenius+TX, Cedecea dravisae+TX, Cellulomonas flavigena+TX, Chaetomium cochliodes (Nova-Cide®)+TX, Chaetomium globosum (Nova-Cide®)+TX, Chromobacterium subtsugae strain PRAA4-1T (Grandevo®)+TX, Cladosporium cladosporioides+TX, Cladosporium oxysponm+TX, Cladosporium chlorocephalum+TX, Cladosporium spp.+TX, Cladosporium tenuissimum+TX, Clonostachys rosea (EndoFine®)+TX, Colletotrichum acutatum+TX, Coniothyrium minitans (Cotans WG®)+TX, Coniothyrium spp.+TX, Cryptococcus albidus (YIELDPLUS®)+TX, Cryptococcus humicola+TX, Cryptococcus infirmominiatus+TX, Cryptococcus laurentii+TX, Cryptophlebia leucotreta granulovirus (Cryptex®)+TX, Cupriavidus campinensis+TX, Cydia pomonella granulovirus (CYD-X®)+TX, Cydia pomonella granulovirus (Madex®+TX, Madex Plus®+TX, Madex Max/Carpovirusine®)+TX, Cylindrobasidium laeve (Stumpout®)+TX, Cylindrocladium+TX, Debaryomyces hansenii+TX, Drechslera hawaiinensis+TX, Enterobacter doacae+TX, Enterobacteriaceae+TX, Entomophtora virulenta (Vektor®)+TX, Epicoccum nigrum+TX, Epicoccum purpureascens+TX, Epicoccum spp.+TX, Filobasidium forifbrme+TX, Fusarium acuminatum+TX, Fusarium chlamydosporum+TX, Fusarium oxysporum (Fusaclean®/Bofox C®)+TX, Fusarium proliferatum+TX, Fusarium spp.+TX, Galactomyces geotichum+TX, Gliocladium catenulatum (Primastop®+TX, Prestop®)+TX, Gliocladium roseum+TX, Gliocladium spp. (SoilGard®)+TX, Gliocladium virens (Soilgard®)+TX, Granulovirus (Granupom®)+TX, Halobacillus halophilus+TX, Halobacillus litoralis+TX, Halobacillus trueperi+TX, Halomonas spp.+TX, Halomonas subglaciescola+TX, Halovibrio variabilis+TX, Hanseniaspora uvarum+TX, Helicoverpa armigera nucleopolyhedrovirus (Helicovex®)+TX, Helicoverpa zea nuclear polyhedrosis virus (Gemstar®)+TX, Isoflavone-formononetin (Myconate®)+TX, Kloeckere apiculata+TX, Kloeckera spp.+TX, Lagenidium giganteum (Laginex®)+TX, Lecanicillium longisporum (Vertiblast®)+TX, Lecanicllium muscarium (Vertikll®)+TX, Lymantria dispar nucleopolyhedrosis virus (Disparvirus®)+TX, Marinococcus halophilus+TX, Meira geulakonigl+TX, Metarhizium anisopliae (Met52®)+TX, Metarhizium anisopilae (Destruxin WP®)+TX, Metschnikowia fruticola (Shemer®)+TX, Metschnikowia pulcherima+TX, Microdochium dimerum (Antibot®)+TX, Micromonospora coerulea+TX, Microsphaeropsis ochracea+TX, Muscodor albus 620 (Muscudor®)+TX, Muscodor roseus strain A3-5+TX, Mycorrhizae spp. (AMykor®+TX, Root Maximizer)+TX, Myrothecium verrucara strain AARC-0255 (DiTera®)+TX, BROS PLUS®+TX, Ophiostoma piliferum strain D97 (Sylvanex®)+TX, Paecilomyces farinosus+TX, Paecilomyces fumosoroseus (PFR-97®+TX, PreFeRal®)+TX, Paecilomyces linacinus (Biostat WP®)+TX, Paecilomyces lilacinus strain 251 (MeloCon WG®)+TX, Paenibacillus polymyxa+TX, Pantoea agglomerans (BlghtBan C9-1®)+TX, Pantoea spp.+TX, Pasteuria spp. (Econem)+TX, Pasteuria nishizawae+TX, Penicillium aurantiogriseum+TX, Penicillium billai (Jumpstart®+TX, TagTeam®)+TX, Penicillium brevicompactum+TX, Penicillium frequentans+TX, Penicillium griseofulvum+TX, Penicillium purpurogenum+TX, Penicillium spp.+TX, Penicillium viridicatum+TX, Phlebiopsis gigantean (Rotstop®)+TX, phosphate solubilizing bacteria (Phosphomeale)+TX, Phytophthora cryptogea+TX, Phytophthora palmivora (Devine®)+TX, Pichia anomala+TX, Pichia guilermondii+TX, Pichia membranaefaciens+TX, Pichia onychis+TX, Pichia stipites+TX, Pseudomonas aeruginosa+TX, Pseudomonas aureofasciens (Spot-Less Biofungicide®)+TX, Pseudomonas cepacia+TX, Pseudomonas chlroraphis (AtEze®)+TX, Pseudomonas corrugate+TX, Pseudomonas fluorescens strain A506 (BlightBan A506®)+TX, Pseudomonas putida+TX, Pseudomonas reactans+TX, Pseudomonas spp.+TX, Pseudomonas syringae (Bio-Save®)+TX, Pseudomonas viridiflava+TX, Pseudomonas fluorescens (Zequanox®)+TX, Pseudozyma flocculosa strain PF-A22 UL (Sporodex L®)+TX, Puccinla canaliculata+TX, Puccinla thlaspeos (Wood Warrior)+TX, Pythium paroecandrum+TX, Pythium olgandrum (Polygandron®+TX, Polyversum)+TX, Pythium periplocum+TX, Rhanella aquatilis+TX, Rhanella spp.+TX, Rhizobia (Dormal®+TX, Vault®)+TX, Rhizoctonia+TX, Rhodococcus globerulus strain AQ719+TX, Rhodosporidium diobovatum+TX, Rhodosporidium toruloides+TX, Rhodotorula spp.+TX, Rhodotorula glutinis+TX, Rhodotorula graminis+TX, Rhodotorula mucilagnosa+TX, Rhodotorula rubra+TX, Saccharomyces cerevislae+TX, Salinococcus roseus+TX, Sclerotinia minor+TX, Sclerotinia minor (SARRITOR®)+TX, Scytalidium spp.+TX, Scytalidium uredinicola+TX, Spodoptera exigua nuclear polyhedrosis virus (Spod-X®+TX, Spexit®)+TX, Serratia marcescens+TX, Serratia plymuthica+TX, Serratia spp.+TX, Sordaria fimicola+TX, Spodoptera littoralis nucleopolyhedrovirus (Littovitr®)+TX, Sporobolomyces roseus+TX, Stenotrophomonas maltophilia+TX, Streptomyces ahygroscopicus+TX, Streptomyces albaduncus+TX, Streptomyces exfollates+TX, Streptomyces galbus+TX, Streptomyces griseoplanus+TX, Streptomyces griseovkidis (Mycostop®)+TX, Streptomyces lydicus (Actinovate®)+TX, Streptomyces lydicus WYEC-108 (ActinoGrow®)+TX, Streptomyces violaceus+TX, Tilletiopsis minor+TX, Tilletiopsis spp.+TX, Trichoderma asperellum (T34 Biocontrol®)+TX, Trichoderma gamsii (Tenet®)+TX, Trichoderma atrovirkide (Plantmate®)+TX, Trichoderma hamatum TH 382+TX, Trichoderma harzianum rifai (Mycostar®)+TX, Trichoderma harzianum T-22 (Trianum-P®+TX, PlantShield HC®+TX, RootShiekld+TX, Trianum-G®)+TX, Trichoderma harzianum T-39 (Trichodex®)+TX, Trichoderma inhamatum+TX, Trfchoderma koningii+TX, Trichoderma spp. LC 52 (Sentinel®)+TX, Trichoderma lignorum+TX, Trichoderma longibrachlatum+TX, Trichoderma polysporum (Binab T®)+TX, Trchoderma taxi+TX, Trfchoderma virens+TX, Trichoderma virens (formerly Gliocladium virens GL-21) (SoilGuard®)+TX, Trichoderma viride+TX, Trichoderma viride strain ICC 080 (Remedie®)+TX, Trichosporon pullulans+TX, Trichosporon spp.+TX, Trichothecium spp.+TX, Trichothecium roseum+TX, Typhula phacorrhiza strain 94670+TX, Typhula phacorrhiza strain 94671+TX, Ulocladium atrum+TX, Ulocladium oudemansii (Botry-Zen®)+TX, Ustilago maydis+TX, various bacteria and supplementary micronutrients (Natural II®)+TX, various fungi (Millennium Microbes®)+TX, Veticillium chlamydosporum+TX, Verticillium lecanii (Mycotal®+TX, Vertalec®)+TX, Vip3Aa20 (VIPtera®)+TX, Virgibaclillus marismortui+TX, Xanthomonas campestris pv. Poae (Camperico®)+TX, Xenorhabdus bovienii+TX, Xenorhabdus nematophilus; and

Plant extracts including: pine oil (Retenol®)+TX, azadirachtin (Plasma Neem Oil+TX, AzaGuard®+TX, MeemAzal®+TX, Moit-X®+TX, Botanical IGR (Neemazad®+TX, Neemix)+TX, canola oil (Lilly Miller Vegol®)+TX, Chenopodium ambrosloides near ambrosioides (Requiem®)+TX, Chrysanthemum extract (Crisant®)+TX, extract of neem oil (Trilogy®)+TX, essentials oils of Lablatae (Botania®)+TX, extracts of clove rosemary peppermint and thyme oil (Garden insect Killer®)+TX, Glycinebetaine (Greenstim®)+TX, garlic+TX, lemongrass oil (GreenMatch®)+TX, neem oil+TX, Nepeta cataria (Catnip oil)+TX, Nepeta catarina+TX, nicotine+TX, oregano oil (MossBuster)+TX, Pedaliaceae oil (Nematon®)+TX, pyrethrum+TX, Quillaja saponaria (NemaQ®)+TX, Reynoutria sachalinensis (Regalia®+TX, Sakalia®)+TX, rotenone (Eco Roten®)+TX, Rutaceae plant extract (Soleo®)+TX, soybean oil (Ortho Ecosense®)+TX, tea tree oil (Timorex Gold®)+TX, thymus oil+TX, AGNIQUE® MMF+TX, BugOl®+TX, mixture of rosemary sesame pepermint thyme and cinnamon extracts (EF 300®)+TX, mixture of clove rosemary and peppermint extract (EF 400®)+TX, mixture of clove pepermint garlic oil and mint (Soil Shot®)+TX, kaolin (Screen®)+TX, storage glucam of brown algae (Laminarin®); and

pheromones including: blackheaded fireworm pheromone (3M Sprayable Blackheaded Fireworm Pheromone®)+TX, Codling Moth Pheromone (Paramount dispenser-(CM)/Isomate C-Plus®)+TX, Grape Berry Moth Pheromone (3M MEC-GBM Sprayable Pheromone®)+TX, Leafroller pheromone (3M MEC—LR Sprayable Pheromone®)+TX, Muscamone (Snip7 Fly Bait®+TX, Starbar Premium Fly Bait®)+TX, Oriental Fruit Moth Pheromone (3M oriental fruit moth sprayable Pheromone®)+TX, Peachtree Borer Pheromone (Isomate-P®)+TX, Tomato Pinworm Pheromone (3M Sprayable Pheromone®)+TX, Entostat powder (extract from palm tree) (Exosex CM®)+TX, (E+TX,Z+TX,Z)-3+TX,8+TX,11 Tetradecatrienyl acetate+TX, (Z+TX,Z+TX,E)-7+TX,11+TX,13-Hexadecatrienal+TX, (E+TX,Z)-7+TX,9-Dodecadlen-1-yl acetate+TX, 2-Methyl-1-butanol+TX, Calcium acetate+TX, Scenturion®+TX, Biolure®+TX, Check-Mate®+TX, Lavandulyl senecioate; and

Macrobials including: Aphelinus abdominalis+TX, Aphidius ervi (Aphelinus-System®)+TX, Acerophagus papaya+TX, Adalia bipunctata (Adalia-System®)+TX, Adalia bipunctata (Adaline®)+TX, Adalla bipunctata (Aphidalia®)+TX, Ageniaspis citricola+TX, Ageniaspis fuscicollis+TX, Amblyseius andersoni (Anderline®+TX, Andersoni-System®)+TX, Amblyseius californicus (Amblyline®+TX, Spical®)+TX, Amblyseius cucumeris (Thripex®+TX, Bugline Cucumeris®)+TX, Amblyseius fallads (Falacis®)+TX, Amblyseius swirskii (Bugline Swirski®+TX, Swirskii-Mite®)+TX, Amblyseius womersleyi (NomerMite®)+TX, Amitus hesperidum+TX, Anagrus atomus+TX, Anagyrus fusdventris+TX, Anagyrus kamali+TX, Anagyrus loecki+TX, Anagyrus pseudococci (Citripar®)+TX, Anicetus benefices+TX, Anisopteromalus calandrae+TX, Anthocaris nemoralis (Anthocoris-System®)+TX, Aphelinus abdominalis (Apheline®+TX, Aphiline®)+TX, Aphelinus asychis+TX, Aphidius coleman (Aphipar®)+TX, Aphidius ervi (Ervipar®)+TX, Aphidius gifuensis+TX, Aphidius mabricariae (Aphipar-M®)+TX, Aphidoletes aphidimyza (Aphidend®)+TX, Aphidoletes aphidimyza (Aphidoline®)+TX, Aphytis lingnanensis+TX, Aphytis melinus+TX, Aprostocetus hagenowii+TX, Atheta coriaria (Staphyline®)+TX, Bombus spp.+TX, Bombus terrestris (Natupol Beehive®)+TX, Bombus terrestris (Beeline®+TX, Tripol®)+TX, Cephalonomia stephanoderis+TX, Chilocorus nigritus+TX, Chrysoperla carnea (Chrysoline®)+TX, Chrysoperla carnea (Chrysopa®)+TX, Chrysoperla rufilabris+TX, Cirrospilus ingenuus+TX, Cirospilus quadristriatus+TX, Citrostichus phyllocnistoides+TX, Closterocerus chamaeleon+TX, Closterocerus spp.+TX, Coccidoxenoides perminutus (Planopar®)+TX, Coccophagus cowperi+TX, Coccophagus iycimnia+TX, Cotesia flavipes+TX, Cotesia plutellae+TX, Cryptolaemus montrouzieri (Cryptobug®+TX, Cryptoline®)+TX, Cybocephalus nipponicus+TX, Dacnusa sibirica+TX, Dacnusa sibirica (Minusa®)+TX, Diglyphus isaea (Diminex®)+TX, Delphastus catalinae (Delphastus®)+TX, Delphastus pusillus+TX, Diachasmimorpha krausii+TX, Diachasmimorpha longicaudata+TX, Diaparsis jucunda+TX, Diaphorencyrttus aligarhensis+TX, Diglyphus isaea+TX, Diglyphus saea (Miglyphus®+TX, Digline)+TX, Dacnusa sibirica (DacDigline®+TX, Minex®)+TX, Diversinervus spp.+TX, Encarsia citrina+TX, Encarsia formosa (Encarsia Max®+TX, Encarline®+TX, En-Strip®)+TX, Eretmocerus eremicus (Enermix®)+TX, Encarsia guadeloupae+TX, Encarsia haitiensis+TX, Episyrphus balteatus (Syrphidend®)+TX, Eretmoceris siphonini+TX, Eretmocerus californicus+TX, Eretmocerus eremicus (Ercal®+TX, Eretline E®)+TX, Eretmocerus eremicus (Bemimix®)+TX, Eretmocerus hayati+TX, Eretmocerus mundus (Bemipar®+TX, Eretline M®)+TX, Eretmocerus siphonini+TX, Exochomus quadripustulatus+TX, Feltiella acarisuga (Spidend®)+TX, Feltiella acansuga (Feltiline®)+TX, Fopius arisanus+TX, Fopius ceratitivorus+TX, Formononetin (Wirless Beehome®)+TX, Franklinothrips vespiformis (Vespop®)+TX, Galendromus occidentalis+TX, Goniozus legneri+TX, Habrobracon hebetor+TX, Harmonia axyridis (HarmoBeetle®)+TX, Heterorhabditis spp. (Lawn Patrol®)+TX, Heterorhabditis bacteriophora (NemaShield HB®+TX, Nemaseek®+TX, Terranem-Nam®+TX, Terranem®+TX, Larvanemre+TX, B-Greene+TX, NemAttack®+TX, Nematop®)+TX, Heterorhabditis megidis (Nemasys H®+TX, BioNem H+TX, Exhibitline Hm®+TX, Larvanem-M®)+TX, Hippodamia convergens+TX, Hypoaspis acuieifer (Acuieifer-System®+TX, Entomite-A®)+TX, Hypoaspis miles (Hypoline M®+TX, Entomite-M®)+TX, Lbalia leucospoides+TX, Lecanoideus floccissimus+TX, Lemophagus errabundus+TX, Leptomastidea abnormis+TX, Leptomastix dactylopil (Leptopar®)+TX, Leptomastix epona+TX, Lindorus lophanthae+TX, Lipolexis oregmae+TX, Lucilia caesar (Natufly®)+TX, Lysiphlebus testaceipes+TX, Macrolophus caliginosus (Mirical-N®+TX, Macroline C®+TX, Mirical®)+TX, Mesoseiulus longipes+TX, Metaphycus flavus+TX, Metaphycus lounsburyi+TX, Micromus angulatus (Milacewing®)+TX, Microterys flavus+TX, Muscidifurax raptorellus and Spalangia cameroni (Biopar®)+TX, Neodryinus typhlocybae+TX, Neoseiulus californicus+TX, Neoseiulus cucumeris (THRYPEX®)+TX, Neoseiulus fallacis+TX, Nesideocoris tenuis (NesidioBug®+TX, Nesibug®)+TX, Ophyra aenescens (Biofly®)+TX, Orius insidiosus (Thripor-I®+TX, Orline I®)+TX, Orius laevigatus (Thripor-L®+TX, Orline I®)+TX, Orius majusculus (Oriline M®)+TX, Orius strigicollis (Thripor-S®)+TX, Pauesia juniperorum+TX, Pedioblus foveolatus+TX, Phasmarhabdtis hermaphrodita (Nemaslug®)+TX, Phymastichus coffea+TX, Phytoseiulus macropilus+TX, Phytoseiulus persimilis (Spidex®+TX, Phytoline P®)+TX, Podisus maculiventris (Podisus®)+TX, Pseudacteon curvatus+TX, Pseudacteon obtusus+TX, Pseudacteon tricuspis+TX, Pseudaphycus maculipennis+TX, Pseudleptomastix mexicana+TX, Psyllaephagus pilosus+TX, Psyttalia concolor (complex)+TX, Quadrastichus spp.+TX, Rhyzobius lophanthae+TX, Rodolia cardinalis+TX, Rumina decollate+TX, Semielacher petiolatus+TX, Sitobion avenae (Ervibank®)+TX, Steinernema carpocapsae (Nematac C®+TX, Millenium®+TX, BioNem C®+TX, NemAttack®+TX, Nemastar®+TX, Capsanem®)+TX, Steinernema feltiae (NemaShieldki®+TX, Nemasys F®+TX, BioNem F®+TX, Steinernema-System®+TX, NemAttack®+TX, Nemaplus®+TX, Exhibitline Sf®+TX, Scia-Rid®+TX, Entonem®)+TX, Steinernema kraussei (Nemasys L®+TX, BoNem L®+TX, Exhibitline Srb®)+TX, Steinernema riobrave (BioVector®+TX, BoVektor®)+TX, Steinernema scapterisci (Nematac S®)+TX, Steinernema spp.+TX, Steinernematid spp. (Guardian Nematodes®)+TX, Stethorus punctillum (Stethorus®)+TX, Tamarixia radiate+TX, Tetrastichus setifer+TX, Thripobius semiluteus+TX, Torymus sinensis+TX, Trichogramma brassicae (Tricholine B®)+TX, Trichogramma brassicae (Tricho-Strip®)+TX, Trichogramma evanescens+TX, Trichogramma minutum+TX, Trichogramma ostriniae+TX, Trichogramma platneri+TX, Trichogramma pretiosum+TX, Xanthopimpla stemmator, and

other biologicals including: abscisic acid+TX, bioSea®+TX, Chondrostereum purpureum (Chontrol Paste®)+TX, Colletotrichum gloeosporioides (Collego®)+TX, Copper Octanoate (Cueva®)+TX, Delta traps (Trapline D®)+TX, Erwinia amylovora (Harpin) (ProAct®+TX, Ni-HIBIT Gold CST®)+TX, Fenrri-phosphate (Ferramol®)+TX, Funnel traps (Trapline Y®)+TX, Gallex®+TX, Grower's Secret®+TX, Homo-brassonolide+TX, Iron Phosphate (Lily Miller Worry Free Ferramol Slug & Snail Bait®)+TX, MCP hail trap (Trapline F®)+TX, Microctonus hyperodae+TX, Mycoleptodiscus terrestris (Des-X®)+TX, BioGain®+TX, Aminomite®+TX, Zenox®+TX, Pheromone trap (Thripline Ams®)+TX, potassium bicarbonate (MilStop®)+TX, potassium salts of fatty acids (Sanova®)+TX, potassium silicate solution (Sil-Matrix®)+TX, potassium iodide+potassiumthiocyanate (Enzicur®)+TX, SuffOil-X®+TX, Spider venom+TX, Nosema locustae (Semaspore Organic Grasshopper Control®)+TX, Sticky traps (Trapline YF®+TX, Rebell Amarillo®)+TX and Traps (Takitrapline y+B®)+TX.

The references in brackets behind the active ingredients, e.g. [3878-19-1] refer to the Chemical Abstracts Registry number. The above described mixing partners are known. Where the active ingredients are included in “The Pesticide Manual” [The Pesticide Manual—A World Compendium; Thirteenth Edition; Editor. C. D. S. TomLin; The British Crop Protection Council], they are described therein under the entry number given in round brackets hereinabove for the particular compound; for example, the compound “abamectin” is described under entry number (1). Where “[CCN]” is added hereinabove to the particular compound, the compound in question is included in the “Compendium of Pesticide Common Names”, which is accessible on the internet [A. Wood; Compendium of Pesticide Common Names, Copyright © 1995-2004]; for example, the compound “acetoprole” is described under the internet address http://www.alanwood.net/pesticdes/acetoprole.html.

Most of the active ingredients described above are referred to hereinabove by a so-called “common name”, the relevant “ISO common name” or another “common name” being used in individual cases. If the designation is not a “common name”, the nature of the designation used instead is given in round brackets for the particular compound; in that case, the IUPAC name, the IUPAC/Chemical Abstracts name, a “chemical name”, a “traditional name”, a “compound name” or a “development code” is used or, if neither one of those designations nor a “common name” is used, an “alternative name” is employed. “CAS Reg. No” means the Chemical Abstracts Registry Number.

The active ingredient mixture of the compounds of formula (I) selected from a compound described in one of Tables 1 to 1.19 (below) or Table T1 (below), and an active ingredient as described above are preferably in a mixing ratio of from 100:1 to 1:6000, especially from 50:1 to 1:50, more especially in a ratio of from 20:1 to 1:20, even more especially from 10:1 to 1:10, very especially from 5:1 and 1:5, special preference being given to a ratio of from 2:1 to 1:2, and a ratio of from 4:1 to 2:1 being likewise preferred, above all in a ratio of 1:1, or 5:1, or 5:2, or 5:3, or 5:4, or 4:1, or 4:2, or 4:3, or 3:1, or 3:2, or 2:1, or 1:5, or 2:5, or 3:5, or 4:5, or 1:4, or 2:4, or 3:4, or 1:3, or 2:3, or 1:2, or 1:600, or 1:300, or 1:150, or 1:35, or 2:35, or 4:35, or 1:75, or 2:75, or 4:75, or 1:6000, or 1:3000, or 1:1500, or 1:350, or 2:350, or 4:350, or 1:750, or 2:750, or 4:750. Those mixing ratios are by weight.

The mixtures as described above can be used in a method for controlling pests, which comprises applying a composition comprising a mixture as described above to the pests or their environment, with the exception of a method for treatment of the human or animal body by surgery or therapy and diagnostic methods practised on the human or animal body.

The mixtures comprising a compound of formula (I) selected from one of Tables 1.1 to 1.19 (below), or Table T1 (below), and one or more active ingredients as described above can be applied, for example, in a single “ready-mix” form, in a combined spray mixture composed from separate formulations of the single active ingredient components, such as a “tank-mix”, and in a combined use of the single active ingredients when applied in a sequential manner, i.e. one after the other with a reasonably short period, such as a few hours or days. The order of applying the compounds of formula (I) selected from Tables 1.1 to 1.19 (below) or Table T1 (below), and the active ingredient(s) as described above, is not essential for working the present invention.

The compositions according to the invention can also comprise further solid or liquid auxiliaries, such as stabilizers, for example unepoxidized or epoxidized vegetable oils (for example epoxidized coconut oil, rapeseed oil or soya oil), antifoams, for example silicone oil, preservatives, viscosity regulators, binders and/or tackifiers, fertilizers or other active ingredients for achieving specific effects, for example bactericides, fungicides, nematocides, plant activators, molluscicides or herbicides.

The compositions according to the invention are prepared in a manner known per se, in the absence of auxiliaries for example by grinding, screening and/or compressing a solid active ingredient and in the presence of at least one auxiliary for example by intimately mixing and/or grinding the active ingredient with the auxiliary (auxiliaries). These processes for the preparation of the compositions and the use of the compounds (I) for the preparation of these compositions are also a subject of the invention.

Another aspect of the invention is related to the use of a compound of formula (I) or of a preferred individual compound as defined herein, of a composition comprising at least one compound of formula (I) or at least one preferred individual compound as above-defined, or of a fungicidal or insecticidal mixture comprising at least one compound of formula (I) or at least one preferred individual compound as above-defined, in admixture with other fungicides or insecticides as described above, for controlling or preventing infestation of plants, e.g. useful plants such as crop plants, propagation material thereof, e.g. seeds, harvested crops, e.g. harvested food crops, or non-living materials by insects or by phytopathogenic microorganisms, preferably fungal organisms.

A further aspect of the invention is related to a method of controlling or preventing an infestation of plants, e.g., useful plants such as crop plants, propagation material thereof, e.g. seeds, harvested crops, e.g., harvested food crops, or of non-living materials by insects or by phytopathogenic or spoilage microorganisms or organisms potentially harmful to man, especially fungal organisms, which comprises the application of a compound of formula (I) or of a preferred individual compound as above-defined as active ingredient to the plants, to parts of the plants or to the locus thereof, to the propagation material thereof, or to any part of the non-living materials.

Controlling or preventing means reducing infestation by phytopathogenic or spoilage microorganisms or organisms potentially harmful to man, especially fungal organisms, to such a level that an improvement is demonstrated.

A preferred method of controlling or preventing an infestation of crop plants by phytopathogenic microorganisms, especially fungal organisms, or insects which comprises the application of a compound of formula (I), or an agrochemical composition which contains at least one of said compounds, is foliar application. The frequency of application and the rate of application will depend on the risk of infestation by the corresponding pathogen or insect. However, the compounds of formula (I) can also penetrate the plant through the roots via the soil (systemic action) by drenching the locus of the plant with a liquid formulation, or by applying the compounds in solid form to the soil, e.g. In granular form (soil application). In crops of water rice such granulates can be applied to the flooded rice field. The compounds of formula I may also be applied to seeds (coating) by impregnating the seeds or tubers either with a liquid formulation of the fungicide or coating them with a solid formulation.

A formulation, e.g. a composition containing the compound of formula (I), and, if desired, a solid or liquid adjuvant or monomers for encapsulating the compound of formula (I), may be prepared in a known manner, typically by intimately mixing and/or grinding the compound with extenders, for example solvents, solid carriers and, optionally, surface active compounds (surfactants).

Advantageous rates of application are normally from 5 g to 2 kg of active ingredient (a.i.) per hectare (ha), preferably from 10 g to 1 kg a.i./ha, most preferably from 20 g to 600 g a.i./ha. When used as seed drenching agent, convenient dosages are from 10 mg to 1 g of active substance per kg of seeds.

When the combinations of the present invention are used for treating seed, rates of 0.001 to 50 g of a compound of formula I per kg of seed, preferably from 0.01 to 10 g per kg of seed are generally sufficient.

Suitably, a composition comprising a compound of formula (I) according to the present invention is applied either preventative, meaning prior to disease development or curative, meaning after disease development.

The compositions of the invention may be employed in any conventional form, for example in the form of a twin pack, a powder for dry seed treatment (DS), an emulsion for seed treatment (ES), a flowable concentrate for seed treatment (FS), a solution for seed treatment (LS), a water dispersible powder for seed treatment (WS), a capsule suspension for seed treatment (CF), a gel for seed treatment (GF), an emulsion concentrate (EC), a suspension concentrate (SC), a suspo-emulsion (SE), a capsule suspension (CS), a water dispersible granule (WG), an emulsifiable granule (EG), an emulsion, water in oil (EO), an emulsion, oil in water (EW), a micro-emulsion (ME), an oil dispersion (OD), an oil miscible flowable (OF), an oil miscible liquid (OL), a soluble concentrate (SL), an ultra-low volume suspension (SU), an ultra-low volume liquid (UL), a technical concentrate (TK), a dispersible concentrate (DC), a wettable powder (WP) or any technically feasible formulation in combination with agriculturally acceptable adjuvants.

Such compositions may be produced in conventional manner, e.g. by mixing the active ingredients with appropriate formulation inerts (diluents, solvents, fillers and optionally other formulating ingredients such as surfactants, biocides, anti-freeze, stickers, thickeners and compounds that provide adjuvancy effects). Also conventional slow release formulations may be employed where long lasting efficacy is intended. Particularly formulations to be applied in spraying forms, such as water dispersible concentrates (e.g. EC, SC, DC, OD, SE, EW, EO and the like), wettable powders and granules, may contain surfactants such as wetting and dispersing agents and other compounds that provide adjuvancy effects, e.g. the ondensation product of formaldehyde with naphthalene sulphonate, an alkylarylsulphonate, a lignin sulphonate, a fatty alkyl sulphate, and ethoxylated alkylphenol and an ethoxylated fatty alcohol.

A seed dressing formulation is applied in a manner known per se to the seeds employing the combination of the invention and a diluent in suitable seed dressing formulation form, e.g. as an aqueous suspension or in a dry powder form having good adherence to the seeds. Such seed dressing formulations are known in the art. Seed dressing formulations may contain the single active ingredients or the combination of active ingredients in encapsulated form, e.g. as slow release capsules or microcapsules.

In general, the formulations include from 0.01 to 90% by weight of active agent, from 0 to 20% agriculturally acceptable surfactant and 10 to 99.99% solid or liquid formulation inerts and adjuvant(s), the active agent consisting of at least the compound of formula (I) optionally together with other active agents, particularly microbiocides or conservatives or the like. Concentrated forms of compositions generally contain in between about 2 and 80%, preferably between about 5 and 70% by weight of active agent. Application forms of formulation may for example contain from 0.01 to 20% by weight, preferably from 0.01 to 5% by weight of active agent. Whereas commercial products will preferably be formulated as concentrates, the end user will normally employ diluted formulations.

Whereas it is preferred to formulate commercial products as concentrates, the end user will normally use dilute formulations.

TABLE 1.1
This table discloses 24 specific compounds of the formula (1-1):

| | |
|---|---|
| 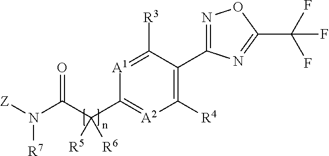 | (T-1) |

wherein n is 0, A¹ is C—R¹, A is C—R², and R², R³, R⁴, and R⁷ are hydrogen, R¹ is fluorine, and Z is as defined below in the Table 1.

Each of Tables 1.2 to 1.19 (which follow Table 1) make available 24 individual compounds of the formula (T-1) in which n, A¹, A^{Z}, R¹, R², R³, R⁴, R⁵, R⁶, and R⁷ are as specifically defined in Tables 12 to 1.19, which refer to Table 1 wherein Z is specifically defined.

TABLE 1

| Compound no. | Z |
|---|---|
| 1.001 | 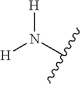 [SMILES: *N] |
| 1.002 | 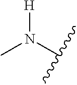 [SMILES: *NC] |
| 1.003 | 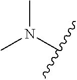 [SMILES: *N(C)C] |
| 1.004 | 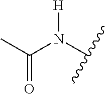 [SMILES: *NC(C)=O] |
| 1.005 | 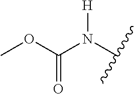 [SMILES: *NC(=O)OC] |
| 1.006 | 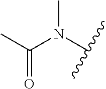 [SMILES: *N(C)C(C)=O] |
| 1.007 | 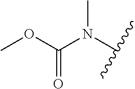 [SMILES: *N(C)C(=O)OC] |
| 1.008 | 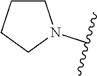 [SMILES: *N1CCCC1] |
| 1.009 | 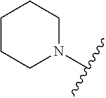 [SMILES: *N1CCCCC1] |
| 1.010 | 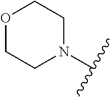 [SMILES: *N1CCOCC1] |
| 1.011 | 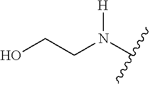 [SMILES: *NCCO] |
| 1.012 | 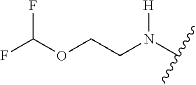 [SMILES: *NCCOC(F)F] |
| 1.013 | 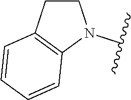 [SMILES: *N1CCc2ccccc21] |
| 1.014 | 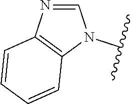 [SMILES: *n1cnc2ccccc21] |
| 1.015 | 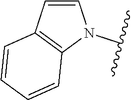 [SMILES: *n1ccc2ccccc21] |
| 1.016 | 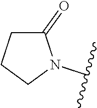 [SMILES: *N1CCCC1=O] |
| 1.017 | 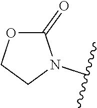 [SMILES: *N1CCOC1=O] |
| 1.018 | 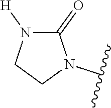 [SMILES: *N1CCNC1=O] |
| 1.019 | 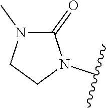 [SMILES: *N1CCN(C)C1=O] |
| 1.020 | 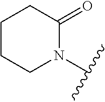 [SMILES: *N1CCCCC1=O] |
| 1.021 | 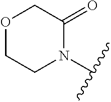 [SMILES: *N1CCOCC1=O] |
| 1.022 | 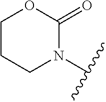 [SMILES: *N1CCCOC1=O] |
| 1.023 | 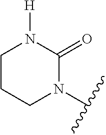 [SMILES: *N1CCCNC1=O] |
| 1.024 | 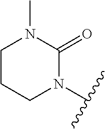 [SMILES: *N1CCCN(C)C1=O] |

Table 1.2: This table discloses 22 specific compounds (ie, compounds 2.002 to 2.009 and 2.011 to 2.024) of formula (T-1) wherein n is 0, A¹ is C—R¹, A² is C—R², and R¹, R², R³, R⁴, and R⁷ are hydrogen, and Z is as defined above in Table 1.
Table 1.3: This table discloses 24 specific compounds of formula (T-1) wherein n is 0, A¹ is C—R¹, A² is C—R², and R², R³, R⁴, and R⁷ are hydrogen, R¹ is chlorine, and Z is as defined above in Table 1.
Table 1.4: This table discloses 24 specific compounds of formula (T-1) wherein n is 0, A¹ is C—R¹, A² is C—R², and R², R³, R⁴, and R⁷ are hydrogen, R¹ is methoxy, and Z is as defined above in Table 1.
Table 1.5: This table discloses 24 specific compounds of formula (T-1) wherein n is 0, A¹ is C—R¹, A2 is C—R², and R², R³, R⁴, and R⁷ are hydrogen, R¹ is methyl, and Z is as defined above in Table 1.
Table 1.6: This table discloses 24 specific compounds of formula (T-1) wherein n is 0, A¹ is C—R¹. A² is C—R², and R¹, R², R¹, and R⁷ are hydrogen. R³ is fluorine, and Z is as defined above in Table 1.
Table 1.7: This table discloses 24 specific compounds of formula (T-1) wherein n is 0, A¹ is C—R¹, A² is C—R², and R³, R⁴, and R⁷ are hydrogen, R¹ and R² are fluorine, and Z is as defined above in Table 1.
Table 1.8: This table discloses 24 specific compounds of formula (T-1) wherein n is 0, A¹ is C—R¹, A² is C—R², and R³, R⁴, and R⁷ are hydrogen, R¹ and R³ are fluorine, and Z is as defined above in Table 1.
Table 1.9: This table discloses 24 specific compounds of formula (T-1) wherein n is 0, A¹ is N, A² is C—R², and R², R³, R⁴, and R⁷ are hydrogen and Z is as defined above in Table 1.
Table 1.10: This table discloses 24 specific compounds of formula (T-1) wherein n is 0, A¹ is N, A² is C—R², and R³, R⁴, and R⁷ are hydrogen, R² is methyl, and Z is as defined above in Table 1.
Table 1.11: This table discloses 24 specific compounds of formula (T-1) wherein n is 0. A¹ is N, A² is N and R³, R⁴, and R⁷ are hydrogen and Z is as defined above in Table 1.
Table 1.12: This table discloses 24 specific compounds of formula (T-1) wherein n is 0, A¹ is C—R¹, A² is C—R², and R¹, R², R³, and R⁴ are hydrogen, R⁷ is methyl, and Z is as defined above in Table 1.
Table 1.13: This table discloses 24 specific compounds of formula (T-1) wherein n is 0, A¹ is C—R¹, A2 is C—R², and R¹, R², R³, and R⁴ are hydrogen, R⁷ is phenylcarbonyl, and Z is as defined above in Table 1.
Table 1.14: This table discloses 24 specific compounds of formula (T-1) wherein n is 0, A¹ is C—R¹, A² is C—R², and R¹, R², R³, and R⁴, are hydrogen, R⁷ is N-tert-butylacetamide, and Z is as defined above in Table 1.
Table 1.15: This table discloses 24 specific compounds of formula (T-1) wherein n is 1, A¹ is C—R¹, A² is C—R², and R¹, R², R³, R⁴, R⁵, R⁶, and R⁷ are hydrogen, and Z is as defined above in Table 1.
Table 1.16: This table discloses 24 specific compounds of formula (T-1) wherein n is 1, A¹ is C—R¹, A² is C—R², and R², R³, R⁴, R⁵, R⁶, and R⁷ are hydrogen, R¹ is fluorine, and Z is as defined above in Table 1.
Table 1.17: This table discloses 24 specific compounds of formula (T-1) wherein n is 1, A¹ is C—R¹, A² is C—R², and R¹, R², R³, R⁵, R⁶, and R⁷ are hydrogen, R³ is fluorine, and Z is as defined above in Table 1.
Table 1.18: This table discloses 24 specific compounds of formula (T-1) wherein n is 1, A¹ is N, A² is C—R², and R², R³, R⁴, R⁵, R⁶, and R⁷ are hydrogen and Z is as defined above in Table 1.
Table 1.19: This table discloses 24 specific compounds of formula (T-1) wherein n is 1, A¹ is C—R¹, A² is C—R², and R¹, R², R³, R⁴, R⁵, and R⁷ are hydrogen, R⁵ is methyl, and Z is as defined above in Table 1.

### EXAMPLES

The Examples which follow serve to illustrate the invention. The compounds of the invention can be distinguished from known compounds by virtue of greater efficacy at low application rates, which can be verified by the person skilled in the art using the experimental procedures outlined in the Examples, using lower application rates if necessary, for example 50 ppm, 12.5 ppm, 6 ppm, 3 ppm, 1.5 ppm, 0.8 ppm or 0.2 ppm.

Compounds of Formula (I) may possess any number of benefits including, inter alia, advantageous levels of biological activity for protecting plants against diseases that are caused by fungi or superior properties for use as agrochemical active ingredients (for example, greater biological activity, an advantageous spectrum of activity, an increased safety profile (including improved crop tolerance), improved physico-chemical properties, or increased biodegradability).

Throughout this description. LC/MS means Liquid Chromatography Mass Spectrometry and the description of the apparatus and the method (Methods A and B) is as follows:

The description of the LC/MS apparatus and the method A is:

SQ Detector 2 from Waters
Ionisation method: Electrospray
Polarity: positive and negative ions

### Capillary (kV) 3.0. Cone (V) 30.00, Extractor (V) 2.00, Source Temperature (° C.) 150, Desolvation

### Temperature (° C.) 350, Cone Gas Flow (L/Hr) 0, Desolvation Gas Flow (L/Hr) 650

Mass range: 100 to 900 Da
DAD Wavelength range (nm): 210 to 500

Method Waters ACQUITY UPLC with the following HPLC gradient conditions:

(Solvent A: Water/Methanol 20:1+0.05% formic acid and Solvent B: Acetonitrile+0.05% formic acid)

| Time (minutes) | A (%) | B (%) | Flow rate (ml/min) |
|---|---|---|---|
| 0 | 100 | 0 | 0.85 |
| 1.2 | 0 | 100 | 0.85 |
| 1.5 | 0 | 100 | 0.85 |

Type of column: Waters ACQUITY UPLC HSS T3; Column length: 30 mm; Internal diameter of column: 2.1 mm; Particle Size: 1.8 micron; Temperature: 60° C.

The description of the LC/MS apparatus and the method B is:

SQ Detector 2 from Waters
ionisation method: Electrospray
Polarity: positive ions

### Capillary (kV) 3.5, Cone (V) 30.00, Extractor (V) 3.00, Source Temperature (° C.) 150, Desolvation

### Temperature (° C.) 400, Cone Gas Flow (L/Hr) 60. Desolvation Gas Flow (L/Hr) 700

Mass range: 140 to 800 Da
DAD Wavelength range (nm): 210 to 400
Method Waters ACQUITY UPLC with the following HPLC gradient conditions
(Solvent A: Water/Methanol 9:1+0.1% formic acid and Solvent B: Acetonitrile+0.1% formic acid)

| Time (minutes) | A (%) | B (%) | Flow rate (ml/min) |
|---|---|---|---|
| 0 | 100 | 0 | 0.75 |
| 2.5 | 0 | 100 | 0.75 |
| 2.8 | 0 | 100 | 0.75 |
| 3.0 | 100 | 0 | 0.75 |

Type of column: Waters ACQUITY UPLC HSS T3; Column length: 30 mm; Internal diameter of column: 2.1 mm; Particle Size: 1.8 micron; Temperature: 60° C.

Where necessary, enantiomerically pure final compounds may be obtained from racemic materials as appropriate via standard physical separation techniques, such as reverse phase chiral chromatography, or through stereoselective synthetic techniques, eg, by using chiral starting materials.

### Formulation Examples

| Wettable powders | a) | b) | c) |
|---|---|---|---|
| active ingredient [compound of formula (I)] | 25% | 50% | 75% |
| sodium lignosulfonate | 5% | 5% | — |
| sodium lauryl sulfate | 3% | — | 5% |
| sodium diisobutylnaphthalenesulfonate | — | 6% | 10% |
| phenol polyethylene glycol ether | — | 2% | — |
| (7-8 mol of ethylene oxide) | | | |
| highly dispersed silicic acid | 5% | 10% | 10% |
| Kaolin | 62% | 27% | — |

The active ingredient is thoroughly mixed with the adjuvants and the mixture s thoroughly ground in a suitable mil, affording wettable powders that can be diluted with water to give suspensions of the desired concentration.

| Powders for dry seed treatment | a) | b) | c) |
|---|---|---|---|
| active ingredient [compound of formula (I)] | 25% | 50% | 75% |
| light mineral oil | 5% | 5% | 5% |
| highly dispersed silicic acid | 5% | 5% | — |
| Kaolin | 65% | 40% | — |
| Talcum | — | — | 20% |

The active ingredient is thoroughly mixed with the adjuvants and the mixture is thoroughly ground in a suitable mill, affording powders that can be used directly for seed treatment.

### Emulsifiable Concentrate

| | |
|---|---|
| active ingredient [compound of formula (I)] | 10% |
| octylphenol polyethylene glycol ether | 3% |
| (4-5 mol of ethylene oxide) | |
| calcium dodecylbenzenesulfonate | 3% |
| castor oil polyglycol ether (35 mol of ethylene oxide) | 4% |
| Cyclohexanone | 30% |
| xylene mixture | 50% |

Emulsions of any required dilution, which can be used in plant protection, can be obtained from this concentrate by dilution with water.

| Dusts | a) | b) | c) |
|---|---|---|---|
| Active ingredient [compound of formula (I)] | 5% | 6% | 4% |
| Talcum | 95% | — | — |
| Kaolin | — | 94% | — |
| mineral filler | — | — | 96% |

Ready-for-use dusts are obtained by mixing the active ingredient with the carrier and grinding the mixture in a suitable mill. Such powders can also be used for dry dressings for seed.

Extruder granules

| | |
|---|---|
| Active ingredient [compound of formula (I)] | 15% |
| sodium lignosulfonate | 2% |
| Carboxymethylcellulose | 1% |
| Kaolin | 82% |

The active ingredient is mixed and ground with the adjuvants, and the mixture is moistened with water. The mixture is extruded and then dried in a stream of air.

Coated granules

| | |
|---|---|
| Active ingredient [compound of formula (I)] | 8% |
| polyethylene glycol (mol. wt. 200) | 3% |
| Kaolin | 89% |

The finely ground active ingredient is uniformly applied, in a mixer, to the kaolin moistened with polyethylene glycol. Non-dusty coated granules are obtained in this manner.

Suspension concentrate

| | |
|---|---|
| active ingredient [compound of formula (I)] | 40% |
| propylene glycol | 10% |
| nonylphenol polyethylene glycol ether (15 mol of ethylene oxide) | 6% |
| Sodium lignosulfonate | 10% |
| Carboxymethylcellulose | 1% |
| silicone oil (in the form of a 75% emulsion in water) | 1% |
| Water | 32% |

The finely ground active ingredient is intimately mixed with the adjuvants, giving a suspension concentrate from which suspensions of any desired dilution can be obtained by dilution with water. Using such dilutions, living plants as well as plant propagation material can be treated and protected against infestation by microorganisms, by spraying, pouring or immersion.

Flowable concentrate for seed treatment

| | |
|---|---|
| active ingredient [compound of formula (I)] | 40% |
| propylene glycol | 5% |
| copolymer butanol PO/EO | 2% |
| tristyrenephenole with 10-20 moles EO | 2% |
| 1,2-benzisothiazolin-3-one (in the form of a 20% solution in | 0.5% |
| water) | |
| monoazo-pigment calcium salt | 5% |
| Silicone oil (in the form of a 75% emulsion in water) | 0.2% |
| Water | 45.3% |

The finely ground active ingredient is intimately mixed with the adjuvants, giving a suspension concentrate from which suspensions of any desired dilution can be obtained by dilution with water. Using such dilutions, living plants as well as plant propagation material can be treated and protected against infestation by microorganisms, by spraying, pouring or immersion.

### Slow-Release Capsule Suspension

28 parts of a combination of the compound of formula I are mixed with 2 parts of an aromatic solvent and 7 parts of toluene diisocyanate/polymethylene-polyphenylisocyanate-mixture (8:1). This mixture is emulsified in a mixture of 1.2 parts of polyvinylalcohol, 0.05 parts of a defoamer and 51.6 parts of water until the desired particle size is achieved. To this emulsion a mixture of 2.8 parts 1,6-diaminohexane in 5.3 parts of water is added. The mixture is agitated until the polymerization reaction is completed.

The obtained capsule suspension is stabilized by adding 0.25 parts of a thickener and 3 parts of a dispersing agent. The capsule suspension formulation contains 28% of the active ingredients. The medium capsule diameter is 8-15 microns.

The resulting formulation is applied to seeds as an aqueous suspension in an apparatus suitable for that purpose.

### List of Abbreviations

DIEA=N-ethyl-N-isopropyl-propan-2-amine

### DIPEA=N,N-diisopropylethylamine

DMA=dimethylacetamide
DMF=dimethylformamide
DMSO=dimethyl sulfoxide
EDCl=1-ethyl-3-(3-dimethylaminopropyl)carbodiimlde
EtOAc=ethyl acetate
EtOH=ethyl alcohol
HCl=hydrochloric acid
HOBt=hydroxybenzotriazole
mp=melting point
MeOH=methyl alcohol
NaOH=sodium hydroxide
TFAA=trifluoroacetic acid anhydride
THF=tetrahydrofuran

### Preparation Examples

### Example 1: This Example Illustrates the Preparation 2-fluoro-N-(2-oxooxazolidin-3-yl)-4-[5-(trifluoromethyl)-1,2,4-oxadiazol-3-yl]benzamide (Compound 1.3 of Table T1)

### Step 1: Preparation of 2-fluoro-4-(N-hydroxycarbamimidoyl)-benzoic Acid

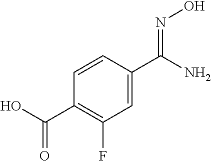 [SMILES: N/C(=N\O)c1ccc(C(=O)O)c(F)c1]

A solution of hydroxylamine hydrochloride (3.0 g) in water (20 mL) was added at room temperature to a stirred solution of 4-cyano-2-fluorobenzoic acid (3.52 g, 21.3 mmol) in ethanol (35 mL), followed by dropwise addition of potassium carbonate (1.60 g). Then 8-hydroxyquinoline (0.041 g, 028 mmol) was added. The resulting thick suspension was heated to reflux for 3 hours to obtain a yellow solution. After removal of ethanol, under reduced pressure, the residue was acidified with 2N HCl to pH 3. The while precipitate was filtered, washed with water and dried under reduced pressure at 50° C. to yield 2-fluoro-4-(N-hydroxycarbamimidoyl)-benzoic acid as beige solid. mp: >250° C. ¹H NMR (400 MHz, DMSO-d6) δ ppm: 13.22 (s, 1H), 10.00 (s, 1H), 7.85 (t, 1H), 7.63 (m, 1H), 7.54-7.61 (m, 1H).

### Step 2: Preparation of 2-fluoro-4-(5-(trifluoromethyl)-1,2,4-oxadiazol-3-yl)-benzoic Acid

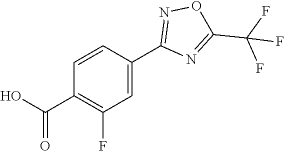 [SMILES: O=C(O)c1ccc(-c2noc(C(F)(F)F)n2)cc1F]

Trifluoroacetic anhydride (4.1 mL) was added dropwise to a stirred suspension of 2-fluoro-4-(N-hydroxycarbamimidoyl)-benzoic acid (3.80 g, 19.0 mmol) in THF (77 mL) at 10 to 15° C. The beige suspension was warmed to room temperature and stirred overnight. After evaporation, the crude product was stirred with heptane/ethylacetate (95:5), filtered and dried under reduced pressure at 50° C. to yield 2-fluoro-4-(5-(trifluoromethyl)-1,2,4-oxadiazol-3-yl)-benzoic acid as yellow solid. mp: 175-177° C. ¹H NMR (400 MHz, DMSO-de) δ ppm: 13.55 (s, 1H), 8.12 (t, 1H), 8.00 (d, 1H), 7.94 (d, 1H).

### Step 3: Preparation of 2-fluoro-4-(5-(trifluoromethyl)-[1.2.4]oxadiazol-3-yl)-benzoyl chloride

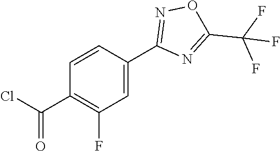 [SMILES: O=C(Cl)c1ccc(-c2noc(C(F)(F)F)n2)cc1F]

To a white suspension consisting of 2-fluoro-4-(5-(trifluoromethyl)-1,2,4-oxadiazol-3-yl)benzoic acid (3.6 g, 13.0 mmol) and CH₂Cl₂ (130 mL) at room temperature was added thionyl chloride (1.51 mL) dropwise. The resulting suspension was heated to reflux and stirred for 3 hours, to obtain a yellow solution. The solvent was evaporated under reduced pressure at 30° C. to yield 2-fluoro-4-(5-(trifluoromethyl)-[1,2,4]oxadiazol-3-yl)-benzoyl chloride as yellowish solid that was used directly without purification. ¹H NMR (400 MHz, CDCl₃) δ ppm: 8.26 (t, 1H), 8.07 (m, 1H), 7.99 (m, 1H).

### Step 4: Preparation of 2-fluoro-N-(2-oxooxazolidin-3-yl)-4-[5-(trifluoromethyl)-1,2,4-oxadiazol-3-yl]benzamide

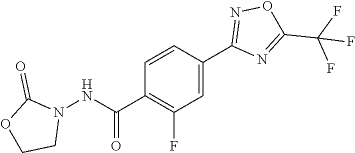 [SMILES: O=C(NN1CCOC1=O)c1ccc(-c2noc(C(F)(F)F)n2)cc1F]

To a screw-cap vial containing 3-aminooxazolidin-2-one (0.05 g) suspended in CH₂Cl₂ (12 mL) cooled to 0° C. Then triethylamine (0.14 mL) was introduced followed by 2-fluoro-4-[5-(trifluoromethyl)-1,2,4-oxadiazol-3-yl]benzoyl chloride (0.15 g) in one portion. The reaction contents were then poured into a separatory funnel and diluted with CH₂Cl₂ and water. The organic layer was separated and then washed with 1N HCl, 1N NaOH, and brine. The solvent was removed under reduced pressure and the crude residue was purified by flash chromatography over silica gel (heptane:ethyl acetate gradient) to give the title compound as a white solid (mp: 186-189° C.). ¹H NMR (400 MHz, CDCl₃): δ 8.60 (d, 1H), 8.32 (d, 1H), 8.10 (d, 1H), 7.97 (d, 1H), 4.55 (t, 2H), 4.00 (t, 2H). LC/MS retention time=0.85 minutes, 361 (M+H)

### Example 2: This Example Illustrates the Preparation of N-tert-butyl-2-[dimethylamino-[4-[5-(trifluoromethyl)-1,2,4-oxadiazol-3-yl]benzoyl]amino]acetamide (Compound 1.1 of Table T1)

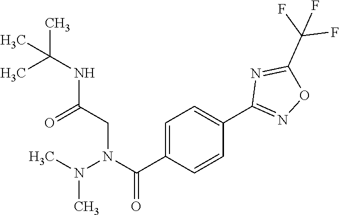 [SMILES: CN(C)N(CC(=O)NC(C)(C)C)C(=O)c1ccc(-c2noc(C(F)(F)F)n2)cc1]

To a screw-cap vial containing 4-[5-(trifluoromethyl)-1,2,4-oxadiazol-3-yl]benzoic acid (0.20 g) suspended in methanol (10 mL) was added 1,1-dimethylhydrazine (0.07 mL) followed by an aqueous solution (35%) of formaldehyde (0.08 mL). After stirring for 30 minutes, tert-butylisocyanide (0.10 mL) was introduced and the reaction was stirred for 20 hours. Upon completion, the volatiles were removed under reduced pressure and the crude contents were poured into a separatory funnel containing EtOAc and water. The organic layer was separated and then washed with brine and over Na₂SO₄. The solvent was removed under reduced pressure and the crude residue was purified by flash chromatography over silica gel (dichiloromethane:ethyl acetate gradient) to give the title compound as an off-color resin. ¹H NMR (400 MHz, CDCl₃): δ 8.13 (d, 2H), 7.68 (d, 1H), 6.66 (brs, 1H), 4.05 (s, 2H), 2.51 (s, 6H), 1.35 (s, 9H). LC/MS retention time=1.64 minutes, 414 (M+H).

### Example 3: This Example Illustrates the Preparation of N′,N′-dimethyl-2-[4-[5-(trifluoromethyl)-1,2,4-oxadiazol-3-yl]phenyl] acetohydrazide (Compound 1.27 of Table T1)

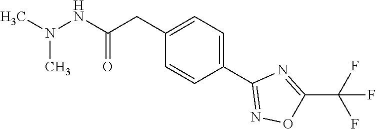 [SMILES: CN(C)NC(=O)Cc1ccc(-c2noc(C(F)(F)F)n2)cc1]

### Step 1: Preparation of N′-hydroxy-4-methyl-benzamidine

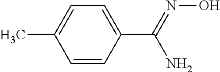

To a stirring suspension of 4-methylbenzonitrile (35 g, 0.29 mol) in ethanol (220 mL) and water (440 mL) was added at room temperature hydroxylamine hydrochloride (41.1 g, 0.58 mol), potassium carbonate (65.4 g, 0.47 mol) and 8-hydroxyquinoline (0.22 g, 1.5 mmol). The reaction mixture was heated at 80° C. for 4 hours. The mixture was cooled to room temperature and diluted with 2N HCl until pH 8. Ethanol was evaporated under reduced pressure then the mixture was filtered, washed with water, and dried under vacuum to afford 39.1 g of N′-hydroxy-4-methyl-benzamidine which was used without further purification.

LC/MS (Method A) retention time=0.23 minutes, 151.0 (M+H).

### Step 2: Preparation of 3-(p-tolyl)-5-(trifluoromethyl-1,2,4-oxadiazole

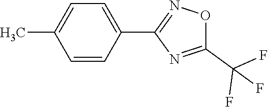 [SMILES: Cc1ccc(-c2noc(C(F)(F)F)n2)cc1]

To a stirring solution of N′-hydroxy-4-methyl-benzamidine (38.7 g, 0.25 mol) in 2-methyltetrahydrofuran (750 mL) was added TFAA at 0° C. The reaction mixture was stirred at 15° C. for two hours then diluted with water. The organic layer was separated, washed successively with an aqueous sodium bicarbonate solution, aqueous ammonium chloride solution, and water. The organic phase was then dried over sodium sulfate, filtered and evaporated to dryness. The crude was subject to flash chromatography over silica gel (750 g prepacked column; eluent heptane/EtOAc 99:1 to 90:10) to afford 54.1 g of 3-(p-tolyl)-5-(trifluoromethyl)-1,2,4-oxadiazole as clear oil, which solidified after storage.

LC/MS (Method A) retention time=1.15 minutes, mass not detected.

¹H NMR (400 MHz, CDCl₃) δ ppm: 8.00 (d, 2H), 7.32 (d, 2H), 2.45 (s, 3H).

¹¹F NMR (400 MHz, CDCl₃) δ ppm: −65.41 (s).

### Step 3a: Preparation of 3-14-(bromomethylphenyl-5-(trifluoromethyl)-1,2,4-oxadiazole

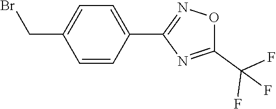 [SMILES: FC(F)(F)c1nc(-c2ccc(CBr)cc2)no1]

A stirring mixture of 3-(p-tolyl)-5-(trifluoromethyl)-1,2,4-oxadiazole (56.0 g, 0.24 mol) and NBS (45.4 g, 0.25 mol) In tetrachloromethane (480 mL) under argon was heated to 70° C. AIBN (4.03 g, 24 mmol) was added and the reaction mixture was stirred at 65° C. for 18 hours. The mixture was cooled to 25° C. and diluted with dichloromethane and water. The organic layer was washed with sodium bicarbonate solution, dried over sodium sulfate, filtered and evaporated to dryness. The crude was subject to flash chromatography over silica gel (750 g pre packed column; eluent cyclohehane/EtOAc 100:0 to 95:5) to afford 44.7 g of 3-[4-(bromomethyl)phenyl]-5-(trifluoromethyl)-1,2,4-oxadiazole as a white solid mp: 58-63° C.

¹H NMR (400 MHz, CDCl₃) δ ppm: 8.11 (d, 2H), 7.55 (d, 2H), 4.53 (s, 2H).

¹⁹F NMR (400 MHz, CDCl₃) δ ppm: −65.32 (s).

3-[4-(dibromomethyl)phenyl]-5-(trifluoromethyl)-1,2,4-oxadiazole (see below) was isolated as by-product as white solid (mp 61-66° C.).

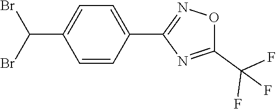 [SMILES: FC(F)(F)c1nc(-c2ccc(C(Br)Br)cc2)no1]

¹H NMR (400 MHz, CDCl₃) δ ppm: 8.15 (d, 2H), 7.73 (d, 2H), 6.68 (s, 1H).

¹⁹F NMR (400 MHz, CDCl₃) δ ppm: −65.34 (s).

### Step 3b: Preparation of 3-[4-(bromomethyl)phenyl]-5-(trifluoromethyl-1,2,4-oxadiazole

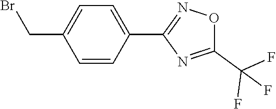 [SMILES: FC(F)(F)c1nc(-c2ccc(CBr)cc2)no1]

To a stirring 1:9 ratio mixture of 3-[4-(bromomethyl)phenyl]-5-(trifluoromethyl)-1,2,4-oxadiazole and 3-[4-(dibromomethyl)phenyl]-5-(trifluoromethyl)-1,2,4-oxadiazole (10.2 g) in acetonitrile (95 mL), water (1.9 mL) and DIPEA (6.20 ml, 35.7 mmol) was added diethylphosphite (4.7 ml, 35.7 mmol) at 5° C. The mixture was stirred at 5-10° C. for two hours, water and 1M HCl were added, and acetonitrile was evaporated under reduced pressure. The white slurry was extracted with dichloromethane and the combined organic layers were dried over sodium sulfate, and filtered. The solvent was removed under reduced pressure and the resultant crude was subject to flash chromatography over silica gel (40 g prepacked column; eluent cyclohexane/EtOAc 99:1 to 9:1) to afford 7.10 g of 3-[4-(bromomethyl)phenyl]-5-(trifluoromethyl)-1,2,4-oxadiazole.

¹H NMR (400 MHz, CDCl₃) δ ppm: 8.11 (d, 2H), 7.55 (d, 2H), 4.53 (s, 2H).

¹⁹F NMR (400 MHz, CDCl₃) δ ppm: −65.32 (s).

### Step 4: Preparation of 2-[4-[5-(trifluoromethyl)-1,2,4-oxadiazol-3-yl]phenyl]acetonitrile

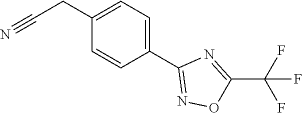 [SMILES: N#CCc1ccc(-c2noc(C(F)(F)F)n2)cc1]

To a solution of 3-[4-(bromomethyl)phenyl]-5-(trifluoromethyl)-1,2,4-oxadiazole (10.0 g, 32.6 mmol) and trimethylsilylformonitrile (4.2 g, 43.9 mmol) in acetonitrile (400 mL) at 0° C. was added a 1M THF solution of tetrabutylammonium hydrofluoride (42.3 mL, 42.3 mmol). The reaction was stirred for 3 hours while reaching room temperature then the reaction contents were diluted with water and extracted with ethyl acetate. The combined organic layer organic was washed with water, brine, dried over Na₂SO₄, and concentrated under reduced pressure. The resultant crude was subject to flash chromatography over silica gel (eluent cyclohexane/EtOAc 8:2) to afford 2-[4-[5-(trifluoromethyl)-1,2,4-oxadiazol-3-yl]phenyl]acetonitrile (7.0 g, 82% yield).

¹H NMR (400 MHz. CDCl₃) δ ppm: 8.15 (m, 2H), 7.55 (m, 2H), 3.88 (m, 2H).

### Step 5: Preparation of methyl 2-[4-[5-(trifluoromethyl-1,2,4-oxadiazol-3-yl]phenyl]acetate

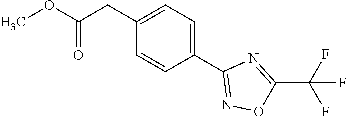 [SMILES: COC(=O)Cc1ccc(-c2noc(C(F)(F)F)n2)cc1]

To a solution of 2-[4-[5-(trifluoromethyl)-1,2,4-oxadiazol-3-yl]phenyl]acetonitrile (7.0 g, 27.6 mmol) in methanol (70 mL) was added chloro(trimethyl)silane (18.0 g, 165.9 mmol) at room temperature then the reaction was heated at 65° C. for 12 hours. After the contents were cooled to room temperature, methanol was removed under reduced pressure and the resultant residue was diluted with water and extracted with ethyl acetate. The organic layer organic was washed with a saturated aqueous NaHCO₃ solution, brine, dried over Na₂SO₄, and concentrated under reduced pressure. The resultant crude residues was subject to flash chromatography over silica gel (eluent cyclohexane/EtOAc 99:1 to 9:1) to afford methyl 2-[4-[5-(trifluoromethyl)-1,2,4-oxadiazol-3-yl]phenyl]acetate (6.5 g, 23 mmol, 71% yield).

¹H NMR (400 MHz, CDCl₃) δ ppm: 8.11 (d, 2H), 7.48 (d, 2H), 3.75 (s, 2H), 3.74 (s, 3H).

Step 6: Preparation of 2-[4-[5-(trifluoromethyl)-1,2,4-oxadiazol-3-yl]phenyl]acetic Acid

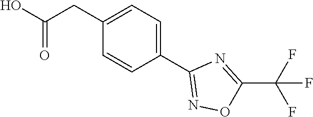 [SMILES: O=C(O)Cc1ccc(-c2noc(C(F)(F)F)n2)cc1]

To a solution of methyl 2-[4-[5-(trifluoromethyl)-1,2,4-oxadiazol-3-yl]phenyl]acetate (2.90 g, 10.1 mmol) in methanol (116 mL) was added dihydroxybarium octahydrate (6.39 g, 20.3 mmol) at 0° C. and the reaction was stirred for 1 hour while reaching room temperature. The reaction contents were then diluted with water and washed with ethyl acetate. The aqueous layer was acidified to pH 2 using a 1M HCl aqueous solution and extracted with ethyl acetate. The combined organic layer organic was washed with water, brine, dried over Na₂SO₄, and concentrated under reduced pressure. The resultant crude residue was subject to flash chromatography over silica gel (eluent cyclohexane/EtOAc 8:2) to afford 2-[4-[5-(trifluoromethyl)-1,2,4-oxadiazol-3-yl]phenyl]acetic acid (2.6 g, 94% yield).

¹H NMR (400 MHz, CDCl₃) δ ppm: 12.49 (brs, 1H), 8.02 (d, 2H), 7.53 (d, 2H), 3.72 (s, 2H).

### Step 7: Preparation of N′,N′-dimethyl-2-[4-[5-(trifluoromethyl)-1,2,4-oxadiazol-3-yl]phenyl] acetohydrazide

To a solution of 2-[4-[5-(trifluoromethyl)-1,2,4-oxadiazol-3-yl]phenyl]acetic acid (0.20 g, 0.7 mmol) in dichloromethane (10 mL) was added EDCl (0.2 g, 1 mmol) and HOBt (0.06 g, 0.4 mmol) then stirred for 10 minutes. To the reaction contents were introduced 1,1-dimethylhydrazine (0.5 g, 0.9 mmol) and trimethylamine (0.3 mL, 2 mmol). After stirring overnight, the resultant residue was diluted with water, stirred for 10 minutes, and extracted with dichloromethane. The organic layer organic was washed with a saturated aqueous NaHCO₃ solution, brine, dried over Na₂SO₄, and concentrated under reduced pressure. The resultant crude was subject to flash chromatography over silica gel (eluent cyclohexane/EtOAc 99:1 to 9:1) to afford N′,N′-dimethyl-2-[4-[5-(trifluoromethyl)-1,2,4-oxadiazol-3-yl]phenyl] acetohydrazide (0.11 g, 50% yield).

¹H NMR (400 MHz, DMSO-d6) δ ppm: 8.47 (m, 1H), 8.01 (m, 2H), 7.47 (m, 2H), 3.39 (m, 2H), 2.46 (m, 6H).

Where necessary, enantiomerically pure final compounds may be obtained from racemic materials as appropriate via standard physical separation techniques, such as reverse phase chiral chromatography, or through stereoselective synthetic techniques, (eg, by using chiral starting materials).

TABLE T1
Melting point (mg) data and/or retention times (RT) for compounds according to Formula (I):

| | | | RT | [M + H] | | mp |
|---|---|---|---|---|---|---|
| Entry | IUPAC name | Structure | (min) | (measured) | Method | (° C.) |
| 1.1 | N-tert-butyl-2- [dimethylamino-[4-[5- (trifluoromethyl)-1,2,4- oxadiazol-3- yl]benzoyl]amino]- acetamide | 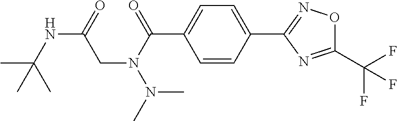 [SMILES: CN(C)N(CC(=O)NC(C)(C)C)C(=O)c1ccc(-c2noc(C(F)(F)F)n2)cc1] | 1.64 | 413 | B | |
| 1.2 | N-[2-(tert-butylamino)- 2-oxo-ethyl]-N-(2- oxooxazolidin-3-yl)-4- [5-(trifluoromethyl)- 1,2,4-oxadiazol-3- yl]benzamide | 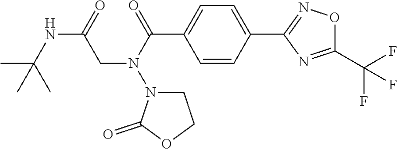 [SMILES: CC(C)(C)NC(=O)CN(C(=O)c1ccc(-c2noc(C(F)(F)F)n2)cc1)N1CCOC1=O] | 1.54 | 456 | B | |
| 1.3 | 2-fluoro-N-(2- oxooxazolidin-3-yl)-4- [5-(trifluoromethyl)- 1,2,4-oxadiazol-3- yl]benzamide | 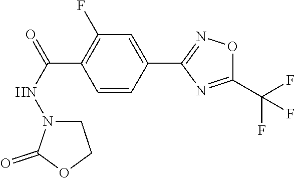 [SMILES: O=C(NN1CCOC1=O)c1ccc(-c2noc(C(F)(F)F)n2)cc1F] | | | | 186.8-188.8 |
| 1.4 | N′-acetyl-2-fluoro-4- [5-(trifluoromethyl)- 1,2,4-oxadiazol-3- yl]benzohydrazide | 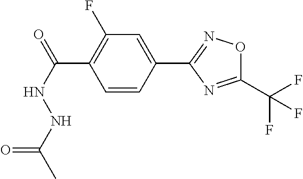 [SMILES: CC(=O)NNC(=O)c1ccc(-c2noc(C(F)(F)F)n2)cc1F] | 0.78 | 333 | A | |
| 1.5 | N-benzoyl-4-[5- (trifluoromethyl)-1,2,4- oxadiazol-3- yl]benzohydrazide | 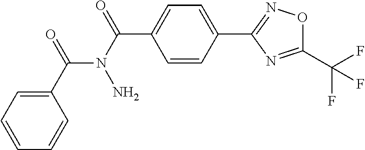 [SMILES: NN(C(=O)c1ccccc1)C(=O)c1ccc(-c2noc(C(F)(F)F)n2)cc1] | | | | 115.3-118.6 |
| 1.6 | methyl N-[[4-[5- (trifluoromethyl)-1,2,4- oxadiazol-3- yl]benzoyl]amino]- carbamate | 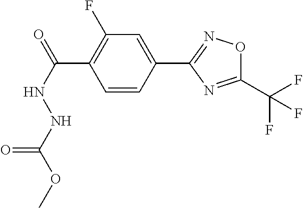 [SMILES: COC(=O)NNC(=O)c1ccc(-c2noc(C(F)(F)F)n2)cc1F] | | | | 165.2-169.9 |
| 1.7 | N-(2-oxo-1-piperidyl)- 4-[5-(trifluoromethyl)- 1,2,4-oxadiazol-3- yl]benzamide | 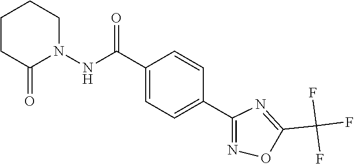 [SMILES: O=C(NN1CCCCC1=O)c1ccc(-c2noc(C(F)(F)F)n2)cc1] | | | | 192-216 |
| 1.8 | N-(3-methyl-2-oxo- imidazolidin-1-yl)-4-[5- (trifluoromethyl)-1,2,4- oxadiazol-3- yl]benzamide | 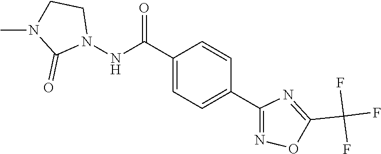 [SMILES: CN1CCN(NC(=O)c2ccc(-c3noc(C(F)(F)F)n3)cc2)C1=O] | | | | 238-245 |
| 1.9 | N-(2,4- dioxoimidazolidin-1- yl)-4-[5- fluoromethyl)-1,2,4- oxadiazoi-3- yl]benzamide | 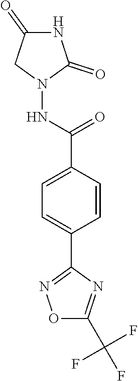 [SMILES: O=C1CN(NC(=O)c2ccc(-c3noc(C(F)(F)F)n3)cc2)C(=O)N1] | | | | 192-196 |
| 1.10 | N-indolin-1-yl-4-[5- (trifluoromethyl)-1,2,4- oxadiazol-3- yl]benzamide | 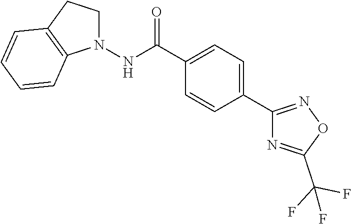 [SMILES: O=C(NN1CCc2ccccc21)c1ccc(-c2noc(C(F)(F)F)n2)cc1] | | | | 167-170 |
| 1.11 | N-indol-1-yl-4-[5- (trifluoromethyl)-1,2,4- oxadiazol-3- yl]benzamide | 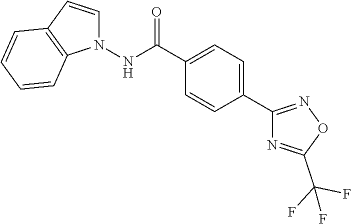 [SMILES: O=C(Nn1ccc2ccccc21)c1ccc(-c2noc(C(F)(F)F)n2)cc1] | | | | 188-190 |
| 1.12 | 2,6-difluoro-N-(2-oxo- 1-piperidyl)-4-[5- (trifluoromethyl)-1,2,4- oxadiazol-3- yl]benzamide | 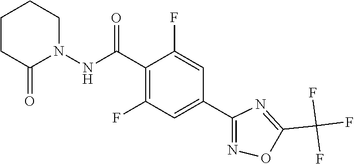 [SMILES: O=C(NN1CCCCC1=O)c1c(F)cc(-c2noc(C(F)(F)F)n2)cc1F] | | | | 205-209 |
| 1.13 | 3-methyl-N-(2-oxo-1- piperidyl)-5-[5- (trifluoromethyl)-1,2,4- oxadiazol-3- yl]pyridine-2- carboxamide | 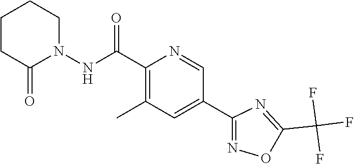 [SMILES: Cc1cc(-c2noc(C(F)(F)F)n2)cnc1C(=O)NN1CCCCC1=O] | | | | 159-162 |
| 1.14 | N-(2-oxo-1-piperidyl)- 2-[4-[5- (trifluoromethyl)-1,2,4- oxadiazol-3- yl]phenyl]acetamide | 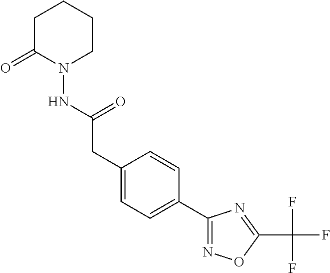 [SMILES: O=C(Cc1ccc(-c2noc(C(F)(F)F)n2)cc1)NN1CCCCC1=O] | | | | 165-170 |
| 1.15 | 2-fluoro-N-morpholino- 4-[5-(trifluoromethyl)- 1,2,4-oxadiazol-3- yl]benzamide | 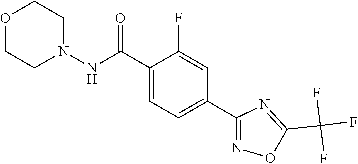 [SMILES: O=C(NN1CCOCC1)c1ccc(-c2noc(C(F)(F)F)n2)cc1F] | | | | 140-160 |
| 1.16 | 3-fluoro-N-morpholino- 4-[5-(trifluoromethyl)- 1,2,4-oxadiazol-3- yl]benzamide | 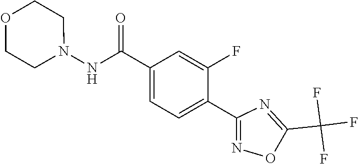 [SMILES: O=C(NN1CCOCC1)c1ccc(-c2noc(C(F)(F)F)n2)c(F)c1] | | | | 140-160 |
| 1.17 | N-morpholino-2-[4-[5- (trifluoromethyl)-1,2,4- oxadiazol-3- yl]phenyl]acetamide | 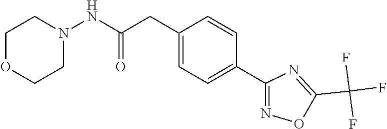 [SMILES: O=C(Cc1ccc(-c2noc(C(F)(F)F)n2)cc1)NN1CCOCC1] | | | | 182-188 |
| 1.18 | N′-(2-hydroxyethyl)-4- [5-(trifluoromethyl)- 1,2,4-oxadiazol-3- yl]benzohydrazide | 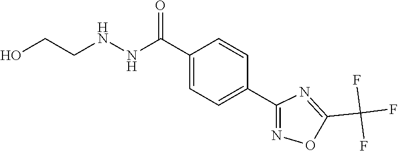 [SMILES: O=C(NNCCO)c1ccc(-c2noc(C(F)(F)F)n2)cc1] | | | | 140-149 |
| 1.19 | N-(2-oxooxazolidin-3- yl)-4-[5- (trifluoromethyl)-1,2,4- oxadiazol-3- yl]benzamide | 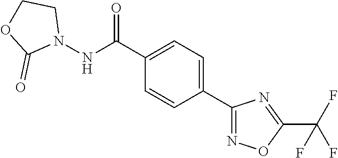 [SMILES: O=C(NN1CCOC1=O)c1ccc(-c2noc(C(F)(F)F)n2)cc1] | | | | 196-200 |
| 1.20 | N-[5- (isopropoxymethyl)-2- oxo-oxazolidin-3-yl]-4- [5-(trifluoromethyl)- 1,2,4-oxadiazol-3- yl]benzamide | 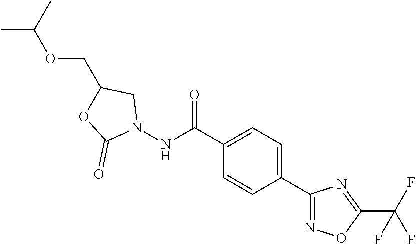 [SMILES: CC(C)OCC1CN(NC(=O)c2ccc(-c3noc(C(F)(F)F)n3)cc2)C(=O)O1] | | | | 192-197 |
| 1.21 | N-(2-oxooxazolidin-3- yl)-2-[4-[5- (trifluoromethyl)-1,2,4- oxadiazol-3- yl]phenyl]acetamide | 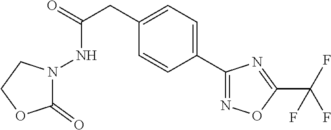 [SMILES: O=C(Cc1ccc(-c2noc(C(F)(F)F)n2)cc1)NN1CCOC1=O] | | | | 184-187 |
| 1.22 | N′-[2- (difluoromethoxy)ethyl]- 4-[5-(trifluoromethyl)- 1,2,4-oxadiazol-3- yl]benzohydrazide | 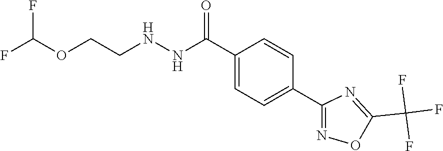 [SMILES: O=C(NNCCOC(F)F)c1ccc(-c2noc(C(F)(F)F)n2)cc1] | 0.97 | 367 | A | |
| 1.23 | 2-fluoro-N-(2-oxo-1- piperidyl)-4-[5- (trifluoromethyl)-1,2,4- oxadiazol-3- yl]benzamide | 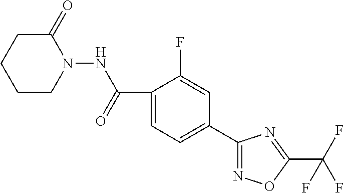 [SMILES: O=C(NN1CCCCC1=O)c1ccc(-c2noc(C(F)(F)F)n2)cc1F] | | | | 120-136 |
| 1.24 | 3-fluoro-N-(2-oxo-1- piperidyl)-4-[5- (trifluoromethyl)-1,2,4- oxadiazol-3- yl]benzamide | 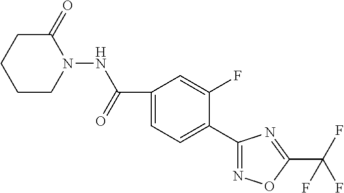 [SMILES: O=C(NN1CCCCC1=O)c1ccc(-c2noc(C(F)(F)F)n2)c(F)c1] | | | | 180-200 |
| 1.25 | 3-fluoro-N-(2- oxooxazolidin-3-yl)-4- [5-(trifluorormethyl)- 1,2,4-oxadiazol-3- yl]benzamide | 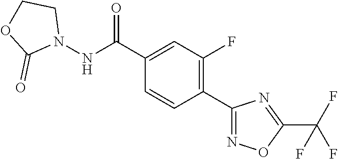 [SMILES: O=C(NN1CCOC1=O)c1ccc(-c2noc(C(F)(F)F)n2)c(F)c1] | | | | 146-166 |
| 1.26 | 2-[2-fluoro-4-[5- (trifluoromethyl)-1,2,4- oxadiazol-3-yl]phenyl]- N′,N′-dimethyl- acetohydrazide | 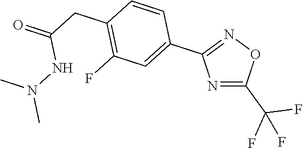 [SMILES: CN(C)NC(=O)Cc1ccc(-c2noc(C(F)(F)F)n2)cc1F] | | | | 156-158 |
| 1.27 | N′,N′-dimethyl-2-[4- [5-(trifluoromethyl)- 1,2,4-oxadiazol-3- yl]phenyl]- acetohydrazide | 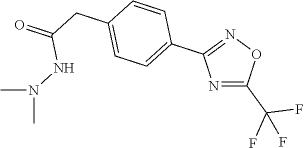 [SMILES: CN(C)NC(=O)Cc1ccc(-c2noc(C(F)(F)F)n2)cc1] | | | | 180-182 |
| 1.28 | 2-[2-fluoro-4-[5- (trifluoromethyl)-1,2,4- oxadiazoi-3-yl]phenyl]- N-morpholino- acetamide | 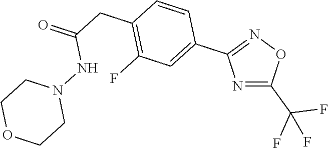 [SMILES: O=C(Cc1ccc(-c2noc(C(F)(F)F)n2)cc1F)NN1CCOCC1] | | | | 201-203 |
| 1.29 | 2-[2-fluoro-4-[5- (trifluoromethyl)-1,2,4- oxadiazol-3-yl]phenyl]- N-(2-oxo-1- piperidyl)acetamide | 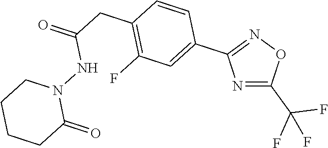 [SMILES: O=C(Cc1ccc(-c2noc(C(F)(F)F)n2)cc1F)NN1CCCCC1=O] | | | | 154-156 |
| 1.30 | 2-[3-fluoro-4-[5- (trifluoromethyl)-1,2,4- oxadiazol-3-yl]phenyl]- N′,N′-dimethyl- acetohydrazide | 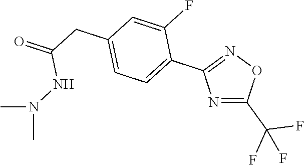 [SMILES: CN(C)NC(=O)Cc1ccc(-c2noc(C(F)(F)F)n2)c(F)c1] | | | | 163-165 |
| 1.31 | 2-[3-fluoro-4-[5- (trifluoromethyl)-1,2,4- oxadiazol-3-yl]phenyl]- N-morpholino- acetamide | 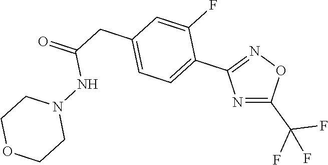 [SMILES: O=C(Cc1ccc(-c2noc(C(F)(F)F)n2)c(F)c1)NN1CCOCC1] | | | | 207-209 |
| 1.32 | 2-[3-flouro-4-[5- (trifluoromethyl)-1,2,4- oxadiazol-3-yl]phenyl]- N-(2-oxo-1- piperidyl)acetamide | 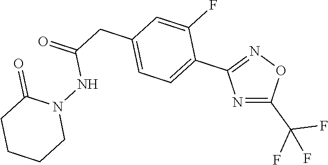 [SMILES: O=C(Cc1ccc(-c2noc(C(F)(F)F)n2)c(F)c1)NN1CCCCC1=O] | | | | 161-163 |

### Biological Examples

### General Examples of Leaf Disk Tests in Well Plates:

Leaf disks or leaf segments of various plant species are cut from plants grown in a greenhouse. The cut leaf disks or segments are placed in multiwell plates (24-well format) onto water agar. The leaf disks are sprayed with a test solution before (preventative) or after (curative) inoculation. Compounds to be tested are prepared as DMSO solutions (max. 10 mg/ml) which are diluted to the appropriate concentration with 0.025% Tween20 just before spraying. The inoculated leaf disks or segments are incubated under defined conditions (temperature, relative humidity, light, etc.) according to the respective test system. A single evaluation of disease level is carried out 3 to 14 days after inoculation, depending on the pathosystem. Percent disease control relative to the untreated check leaf disks or segments is then calculated.

### General Examples of Liquid Culture Tests in Well Plates:

Mycella fragments or conidia suspensions of a fungus prepared either freshly from liquid cultures of the fungus or from cryogenic storage, are directly mixed into nutrient broth. DMSO solutions of the test compound (max. 10 mg/ml) are diluted with 0.025% Tween20 by a factor of 50 and 10 μl of this solution is pipetted into a microtiter plate (96-well format). The nutrient broth containing the fungal spores/mycelia fragments is then added to give an end concentration of the tested compound. The test plates are incubated in the dark at 24° C., and 96% relative humidity. The inhibition of fungal growth is determined photometrically after 2 to 7 days, depending on the pathosystem, and percent antifungal activity relative to the untreated check is calculated.

### Example 1: Fungicidal Activity Against Puccinia recondita f. sp. tritici/Wheat/Leaf Disc Preventative (Brown Rust)

Wheat leaf segments cv. Kanzler are placed on agar in multiwell plates (24-well format) and sprayed with the formulated test compound diluted in water. The leaf disks are inoculated with a spore suspension of the fungus 1 day after application. The inoculated leaf segments are incubated at 19° C. and 75% rh under a light regime of 12 h light/12 h darkness in a climate cabinet and the activity of a compound is assessed as percent disease control compared to untreated when an appropriate level of disease damage appears in untreated check leaf segments (7-9 days after application).

The following compounds gave at least 80% control of Puccinia recondita f. sp. tritici at 200 ppm when compared to untreated control under the same conditions, which showed extensive disease development:

Compounds (from Table T1) 1.1, 1.2, 1.3, 1.4, 1.5, 1.6, 1.7, 1.8, 1.10, 1.11, 1.14, 1.15, 1.16, 1.17, 1.18, 1.19, 1.21, 1.22, 1.23, 1.24, 1.25, 1.26, 1.27, 1.29, 1.30, and 1.32.

### Example 2: Fungicidal Activity Against Puccinia recondita f. sp. tritici/Wheat/Leaf Disc Curative (Brown Rust

Wheat leaf segments cv. Kanzler are placed on agar in multiwell plates (24-well format). The leaf segments are inoculated with a spore suspension of the fungus. Plates are stored in darkness at 19° C., and 75% rh. The formulated test compound diluted in water is applied 1 day after inoculation. The leaf segments are incubated at 19° C., and 75% rh under a light regime of 12 h light/12 h darkness in a climate cabinet and the activity of a compound is assessed as percent disease control compared to untreated when an appropriate level of disease damage appears in untreated check leaf segments (6-8 days after application).

The following compounds gave at least 80% control of Puccinia recondita f. sp. tritic at 200 ppm when compared to untreated control under the same conditions, which showed extensive disease development:

Compounds (from Table T1) 1.1, 1.3, 1.4, 1.6, 1.9, 1.14, 1.15, 1.17, 1.18, 1.19, 1.21, 1.22, 1.23, 1.24, 1.25, 1.26, 1.27, 1.28, 1.29, 1.30, 1.31, and 1.32.

### Example 3: Fungicidal Activity Against Phakopsora pachyrhizi/Soybean/Leaf Disc Preventative (Asian Soybean Rust)

Soybean leaf disks are placed on water agar in multiwell plates (24-well format) and sprayed with the formulated test compound diluted in water. One day after application leaf discs are inoculated by spraying a spore suspension on the lower leaf surface. After an incubation period in a climate cabinet of 24-36 hours in darkness at 20° C., and 75% rh leaf disc are kept at 20° C. with 12 h light/day and 75% rh. The activity of a compound is assessed as percent disease control compared to untreated when an appropriate level of disease damage appears in untreated check leaf disks (12-14 days after application).

The following compounds gave at least 80% control of Phakopsora pachyrhizi at 200 ppm when compared to untreated control under the same conditions, which showed extensive disease development:

Compounds (from Table T1) 1.1, 1.2, 1.3, 1.4, 1.6, 1.7, 1.8, 1.9, 1.14, 1.15, 1.16, 1.17, 1.19, 1.20, 1.21, 1.22, 1.23, 1.24, 1.26, and 1.27.

### Example 4: Fungicidal Activity Against Glomerella lagenarium (Colletotrichum lagenarium) Liquid Culture/Cucumber/Preventative (Anthracnose)

Conidia of the fungus from cryogenic storage are directly mixed into nutrient broth (PDB potato dextrose broth). Alter placing a (DMSO) solution of test compound into a microtiter plate (96-well format), the nutrient broth containing the fungal spores is added. The test plates are incubated at 24° C., and the inhibition of growth is measured photometrically 3-4 days after application.

The following compounds gave at least 80% control of Glomerella lagenarium at 20 ppm when compared to untreated control under the same conditions, which showed extensive disease development:

Compounds (from Table T1) 1.1, 1.3, 1.5, 1.6, 1.10, 1.11, 1.12, 1.15, 1.18, 1.19, 1.20, 1.21, 1.22, 1.23, 1.24, 1.25, 1.26, 1.27, 1.28, 1.29, 1.30, and 1.32.

### Example 5: Fungicidal Activity Against Uromyces viciae-Fabaei Field Bean/Leaf Disc Preventative (Faba-Bean Rust)

Field bean leaf discs are placed on water agar in multiwell plates (96-well format) and 10 μl of the formulated test compound diluted in acetone and a spreader pipetted onto the leaf disc. Two hours after application leaf discs are inoculated by spraying a spore suspension on the lower leaf surface. The leaf discs are incubated in a climate cabinet at 22° C. with 18 hour day and 70% relative humidity. The activity of a compound is assessed as percent disease control compared to untreated when an appropriate level of disease damage appears in untreated check leaf disks (12 days after application).

The following compounds at 100 ppm in the applied formulation give at least 80% disease control in this test when compared to untreated control leaf discs under the same conditions, which show extensive disease development.

Compounds (from Table T1) 1.1, 1.3, 1.4, 1.5, 1.6, 1.7, 1.8, and 1.9.

1. A compound of formula (I): 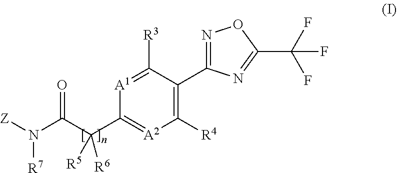
wherein
n represents 0 or 1;
A¹ represents N or CR¹, wherein R¹ is hydrogen, halogen, methyl, ethyl, difluoromethyl, or difluoromethoxy;
A² represents N or CR², wherein R² is hydrogen, halogen, methyl, ethyl, difluoromethyl, or difluoromethoxy;
R³ and R⁴ independently represent hydrogen or halogen;
R⁵ and R⁶ independently represent hydrogen or methyl;
R⁷ is hydrogen, C₁₋₄alkyl, C₁₋₄alkylcarbonyl, C₁₋₄alkylaminocarbonylC₁₋₄alkyl, di-C₁₋₄ alkylaminocarbonylC₁₋₄alkyl, C₁₋₄alkylaminocarbonyl, phenylcarbonyl, or C₃₋₆cycloalkyl;
Z represents —NR⁸R⁹, wherein
R⁸ represents hydrogen or C₁₋₄alkyl;
R⁹ represents hydrogen, C₁₋₄alkyl, C₃₋₆alkenyl, C₃₋₆alkynyl, C₁₋₄haloalkyl, cyanoC₁₋₆alkyl, hydroxyC₁₋₆alkyl, C₁₋₄alkoxyC₁₋₆alkyl, C₁₋₂fluoroalkoxyC₁₋₆alkyl, C₁₋₄alkylcarbonyl, C₁₋₄alkylcarbonylC₁₋₆alkyl, C₁₋₄haloalkylcarbonyl, C₁₋₄haloalkylaminocarbonyl, C₁₋₄alkoxycarbonyl, C₁₋₄alkoxycarbonylC₁₋₆alkyl, C₁₋₄alkylaminocarbonyl, di-C₁₋₄alkylaminocarbonyl, C₁₋₄alkylaminothiocarbonyl, di-C₁₋₄alkylaminothiocarbonyl, C₃₋₈cycloalkyl, C₃₋₈cycloalkylC₁₋₃alkyl, phenyl, phenylC₁₋₃alkyl, phenylcarbonyl, heteroaryl, heteroarylC₁₋₃alkyl, heteroarylcarbonyl, wherein the heteroaryl moiety is a 5- or 6-membered aromatic ring which comprises 1, 2, 3 or 4 heteroatoms individually selected from N, O and S, heterocyclyl, heterocyclylC₁₋₃alkyl, wherein the heterocyclyl moiety is a 4- to 6-membered non-aromatic ring which comprises 1, 2 or 3 heteroatoms individually selected from N, O and S, or heterobicyclyl, wherein the heterobicyclyl moiety is a 7- to 11-membered aromatic, saturated or partially saturated fused ring system which comprises 1, 2 or 3 heteroatoms selected from N, O and S, and wherein any of said cycloalkyl, phenyl, heteroaryl, heterocyclyl or heterobicyclyl moieties is optionally substituted by 1, 2, 3 or 4 substituents, which may be the same or different, selected from R¹⁰;
R¹⁰ represents cyano, amino, halogen, hydroxy, C₁₋₄alkyl, methoxy, ethoxy, allyl, propargyl, difluoromethyl, trifluoromethyl, or difluoromethoxy;
or
R⁸ and R⁹, together with the nitrogen atom to which they are attached, form a 5- or 6-membered non-aromatic heterocyclic ring which optionally additionally comprises 1, 2 or 3 heteroatoms individually selected from N, O and S, wherein the heterocyclic ring is optionally substituted by 1, 2, 3, or 4 substituents, which may be the same or different, selected from R¹¹;
or
R⁸ and R⁹, together with the nitrogen atom to which they are attached, form a 7- to 11-membered heteroaromatic or non-aromatic heterocyclic fused ring system which optionally additionally comprises 1, 2 or 3 heteroatoms individually selected from N, O and S, wherein said heteroaromatic or heterocyclic fused ring system is optionally substituted by 1, 2, 3, or 4 substituents, which may be the same or different, selected from R¹¹; and
R¹¹ represents cyano, amino, halogen, hydroxy, C₁₋₄alkyl, C₁₋₂fluoroalkyl, C₁₋₄alkoxy, C₁₋₂alkoxyC₁₋₄alkyl, allyl, or propargyl and R¹¹ may also represent oxo on a non-aromatic heterocyclic moiety;
or a salt or an N-oxide thereof;
with the proviso that the compound of formula (I) is not:
N′-benzoyl-4-[5-(trifluoromethyl)-1,2,4-oxadiazol-3-yl]benzohydrazide;
1-(2,2,2-trifluoroethyl)-3-[[4-[5-(trifluoromethyl)-1,2,4-oxadiazol-3-yl]benzoyl]amino]urea;
N-morpholin-4-yl-4-[5-(trifluoromethyl)-1,2,4-oxadiazol-3-yl]benzamide;
N′-(3-methylphenyl)-4-[5-(trifluoromethyl)-1,2,4-oxadiazol-3-yl]benzohydrazide;
4-methyl-N′-[4-[5-(trifluoromethyl)-1,2,4-oxadiazol-3-yl]benzoyl]-1,3-thiazole-5-carbohydrazide;
3-methyl-N′-[4-[5-(trifluoromethyl)-1,2,4-oxadiazol-3-yl]benzoyl]thiophene-2-carbohydrazide;
4-[5-(trifluoromethyl)-1,2,4-oxadiazol-3-yl]benzohydrazide; or
4-[5-(trifluoromethyl)-1,2,4-oxadiazol-3-yl]benzohydrazide hydrochloride salt.

2. A compound according to claim 1, wherein A¹ represents N or CR¹, wherein R¹ is selected from hydrogen, halogen, or methyl.

3. A compound according to claim 1, wherein A² represents N or CR², wherein R² is selected from hydrogen, halogen or methyl.

4. A compound according to claim 1, wherein A¹ and A² both represent C—H and R³ and R⁴ both represent hydrogen.

5. A compound according to claim 1, wherein R⁵ and R⁶ both represent hydrogen.

6. A compound according to claim 1, wherein R⁷ is selected from hydrogen, C₁₋₄alkyl, C₁₋₄alkylcarbonyl, C₁₋₄alkylaminocarbonylC₁₋₄alkyl or phenylcarbonyl.

7. A compound according to claim 1, wherein R⁸ represents hydrogen or methyl.

8. A compound according to claim 1, wherein R⁹ is selected from hydrogen, C₁₋₄alkyl, hydroxyC₁₋₄alkyl, C₁₋₄alkoxyC₁₋₆alkyl, C₁₋₂fluoroalkoxyC₁₋₆ alkyl, C₁₋₄alkylcarbonyl, C₁₋₄alkoxycarbonyl, C₃₋₈cycloalkyl, or C₃₋₈cycloalkylC₁₋₃alkyl.

9. A compound according to claim 1, wherein R⁸ and R⁹ together with the nitrogen atom to which they are attached, form a 5-lactam, imidazolinone, imidazolin-dione, oxazolidinone, or morpholine group, which is optionally substituted by 1 or 2 substituents, which may be the same or different, selected from cyano, amino, halogen, hydroxy, and C₁₋₄alkyl.

10. A compound according to claim 1, wherein R⁸ and R⁹ together with the nitrogen atom to which they are attached form an indole or indolene group, which is optionally substituted by 1 or 2 substituents, which may be the same or different, selected from cyano, amino, halogen, hydroxy, and C₁₋₄alkyl.

11. A compound according to claim 1, wherein n is 0.

12. An agrochemical composition comprising a fungicidally effective amount of a compound of formula (I) according to claim 1.

13. The composition according to claim 12, further comprising at least one additional active ingredient and/or an agrochemically-acceptable diluent or carrier.

14. A method of controlling or preventing infestation of useful plants by phytopathogenic microorganisms, wherein a fungicidally effective amount of a compound of formula (I) according to claim 1, or a composition comprising this compound as active ingredient, is applied to the plants, to parts thereof or the locus thereof.

15. Use of a compound of formula (I) according to claim 1 as a fungicide.
